(19) United States
(12) Patent Application Publication
(10) Pub. No.: US 2020/0306373 A1
(43) Pub. Date: Oct. 1, 2020

(54) COMBINATION THERAPY OF TUMOR TARGETED ICOS AGONISTS WITH T-CELL BISPECIFIC MOLECULES

(71) Applicant: Hoffmann-La Roche Inc., Little Falls, NJ (US)

(72) Inventors: Marina BACAC, Schlieren (CH); Tanja FAUTI, Schlieren (CH); Christian KLEIN, Schlieren (CH); Johannes SAM, Schlieren (CH); Pablo UMANA, Schlieren (CH); Ramona MURR, Schlieren (CH); Joerg ZIELONKA, Schlieren (CH); Lucas HABEGGER, Schlieren (CH)

(73) Assignee: Hoffmann-La Roche Inc., Little Falls, NJ (US)

(21) Appl. No.: 16/903,756

(22) Filed: Jun. 17, 2020

Related U.S. Application Data
(63) Continuation of application No. PCT/EP2018/086046, filed on Dec. 20, 2018.

(30) Foreign Application Priority Data
Dec. 21, 2017 (EP) 17209444.3

(51) Int. Cl. A61K 39/395; C07K 16/28

(52) U.S. Cl. CPC A61K 39/3955; C07K 16/2809; C07K 2317/565; C07K 2317/31; C07K 2317/515; C07K 2317/51; C07K 2317/75; C07K 2317/56

(57) ABSTRACT

The present invention relates to agonistic ICOS-binding molecules comprising at least one antigen binding domain that binds to a tumor-associated antigen and their use in combination with T-cell bispecific molecules in the treatment of cancer, the agonistic ICOS-binding molecules as such, pharmaceutical compositions comprising these molecules, and methods of using the same.

### CROSS REFERENCE TO RELATED APPLICATIONS

This application is a continuation of International Application No. PCT/EP2018/086046, Publication No. WO2019/122049, filed Dec. 20, 2018, which claims priority to European Patent Application No. 17209444.3 filed Dec. 21, 2017, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 10, 2020, is named P34548-US_sequence_listing_ST25.txt and is 346 kilobytes in size.

### FIELD OF THE INVENTION

The present invention relates to tumor-targeted agonistic ICOS-binding molecules and their use as immunomodulators in combination with T-cell bispecific molecules in the treatment of cancer.

### BACKGROUND

Modulating immune inhibitory pathways has been a maj or recent breakthrough in cancer treatment. Checkpoint blockade antibodies targeting cytotoxic T-lymphocyte antigen 4 (CTLA-4, YERVOY/ipilimumab) and programmed cell-death protein 1 (PD-1, OPDIVO/nivolumab or KEYTRUDA/pembrolizumab), respective PD-L1 (atezolizumab) have demonstrated acceptable toxicity, promising clinical responses, durable disease control, and improved survival in patients of various tumor indications. However, only a minority of patients experience durable responses to immune checkpoint blockade (ICB) therapy, the remainder of patients show primary or secondary resistance, demonstrating a clear need for regulating additional pathways to provide survival benefit for greater numbers of patients. Thus, combination strategies are needed to improve therapeutic benefit.

ICOS (CD278) is an inducible T-cell co-stimulator and belongs to the B7/CD28/CTLA-4 immunoglobulin superfamily (Hutloff, et al., Nature 1999, 397). Its expression seems to be restricted mainly to T cells with only weak expression on NK cells (Ogasawara et al., J Immunol. 2002, 169 and unpublished own data using human NK cells). Unlike CD28, which is constitutively expressed on T cells, ICOS is hardly expressed on naïve T_{H}1 and T_{H}2 effector T cell populations (Paulos C M et al., Sci Transl Med 2010, 2), but on resting T_{H}17, T follicular helper (T_{FH}) and regulatory T (Treg) cells. However, ICOS is strongly induced on all T cell subsets upon previous antigen priming, respective TCR/CD3-engagement (Wakamatsu et al., Proc Natal Acad Sci USA, 2013, 110).

Signaling through the ICOS pathway occurs upon binding of its ligand, the so-called ICOS-L (B7h, B7RP-1, CD275), which is expressed on B cells, macrophages, dendritic cells, and on non-immune cells treated with TNF-α (Simpson et al., Current Opinion in Immunology 2010, 22). Neither B7-1 nor B7-2, the ligands for CD28 and CTLA4, are able to bind or activate ICOS. Nonetheless, ICOS-L has been shown to bind weakly to both CD28 and CTLA-4 (Yao et al., Immunity 2011, 34). Upon activation, ICOS, a disulfide-linked homodimer, induces a signal through the PI3K and AKT pathways. In contrast to CD28, ICOS has a unique YMFM SH2 binding motif, which recruits a PI3K variant with elevated lipid kinase activity compared to the isoform recruited by CD28. As a consequence, greater production of Phosphatidylinositol (3,4,5)-triphosphate and concomitant increase in AKT signaling can be observed, suggesting an important role of ICOS in T cell survival (Simpson et al., Current Opinion in Immunology 2010, 22).

As reviewed by Sharpe (Immunol Rev., 2009, 229), the ICOS/ICOS ligand pathway has critical roles in stimulating effector T-cell responses, T-dependent B-cell responses, and regulating T-cell tolerance by controlling IL-10 producing Tregs. Moreover, ICOS is important for generation of chemokine (C—X—C motif) receptor 5 (CXCR5)⁺ follicular helper T cells (T_{FH}), a unique T-cell subset that regulates germinal center reactions and humoral immunity. Recent studies in ICOS-deficient mice indicate that ICOS can regulate interleukin-21 (IL-21) production, which in turn regulates the expansion of T helper (Th) type 17 (T_{H}17) cells and T_{FH}. In this context, ICOS is described to bipolarize CD4 T cells towards a T_{H}1-like T_{H}17 phenotype, which has been shown to correlate with improved survival of patients in several cancer indications, including melanoma, early stage ovarian cancer and more (Rita Young, J Clin Cell Immunol. 2016, 7).

ICOS-deficient mice show impaired germinal center formation and have decreased production of IL-10 and IL-17, which become manifest in an impaired development of autoimmunity phenotypes in various disease models, such as diabetes (T_{H}1), airway inflammation (T_{H}2) and EAE neuro-inflammatory models (T_{H}17) (Warnatz et al, Blood 2006). In line with this, human common variable immunodeficiency patients with mutated ICOS show profound hypogammaglobulinemia and a disturbed B-cell homeostatsis (Sharpe, Immunol Rev., 2009, 229). Important to note, that efficient co-stimulatory signaling via ICOS receptor only occurs in T cells receiving a concurrent TCR activation signal (Wakamatsu et al., Proc Natal Acad Sci USA, 2013, 110).

T-cell bispecific (TCB) molecules are appealing immune cell engagers, since they bypass the need for recognition of MHCI-peptide by corresponding T-cell receptors, but enable a polyclonal T-cell response to cell-surface tumor-associated antigens (Yuraszeck et al., Clinical Pharmacology & Therapeutics 2017, 101). CEA CD3 TCB, an anti-CEA/anti-CD3 bispecific antibody, is an investigational, immunoglobulin Gi (IgG1) T-cell bispecific antibody to engage the immune system against cancer. It is designed to redirect T cells to tumor cells by simultaneous binding to human CD3ε on T cells and carcinoembryonic antigen (CEA), expressed by various cancer cells, including CRC (colorectal cancer), GC (gastric cancer), NSCLC (non-small-cell lung cancer) and BC (breast cancer). The cross-linking of T- and tumor cells, leads to CD3/TCR downstream signaling and to the formation of immunologic synapses, T-cell activation, secretion of cytotoxic granules and other cytokines and ultimately to a dose- and time-dependent lysis of tumor cells. Furthermore, CEA CD3 TCB is proposed to increase T-cell infiltration and generate a highly inflamed tumor microenvironment, making it an ideal combination partner for immune checkpoint blockade therapy (ICB), especially for tumors showing primary resistance to ICB because of the lack of sufficient endogenous adaptive and functional immune infiltrate. However, turning-off the brakes by blocking single or multiple inhibitory pathways on T cells might not be sufficient, given the paradoxical expression of several co-stimulatory receptors, such as 4-1BB (CD137), ICOS and OX40 on dysfunctional T cells in the tumor microenvironment (TME).

For ICOS, a growing body of literature actually supports the idea that engaging CD278 on CD4⁺ and CD8⁺ effector T cells has anti-tumor potential. Activating the ICOS-ICOS-L signaling has induced effective anti-tumor responses in several syngeneic mouse models both as monotherapy, as well in the context of anti-CLTA4 treatment, where activation of ICOS downstream signaling increased the efficacy of anti-CTLA4 therapy significantly (Fu T et al., Cancer Res, 2011, 71 and Allison et al., WO2011/041613 A2, 2009). Emerging data from patients treated with anti-CTLA4 antibodies also point to a correlation of sustained elevated levels of ICOS expression on CD4 and CD8 T cells and improved overall survival of tumor patients, e.g. with metastatic melanoma, urothelial, breast or prostate cancer (Giacomo et al., Cancer Immunol Immunother. 2013, 62; Carthon et al., Clin Cancer Res. 2010, 16; Vonderheide et al., Clin Cancer Res. 2010, 16; Liakou et al, Proc Natl Acad Sci USA 2008, 105 and Vonderheide et al., Clin Cancer Res. 2010, 16). Therefore, ICOS positive T effector cells are seen as a positive predictive biomarker of ipilimumab response.

It has been found that a better anti-tumor effect is achieved when an anti-CEA/anti-CD3 bispecific antibody, i.e. a CEA TCB, is combined with a tumor-targeted agonistic ICOS-binding molecule. Given, that ICOS is expressed already at baseline in various tumor indications (Allison et al., 2009, WO2011/041613 A2) and activation of ICOS downstream signaling via PI3K and AKT depends on a simultaneous CD3 trigger for full activity, the combination of a TCB molecule with a tumor-targeted ICOS molecule acts synergistically to induce strong and long-lasting anti-tumor responses. The T-cell bispecific antibody provides the initial TCR activating signalling to T cells, and then the combination with the tumor-targeted agonistic ICOS-binding molecule leads to a further boost of anti-tumor T cell immunity. Thus, we herein describe a novel combination therapy for the treatment of cancer, in particular against tumors expressing CEA (CEA-positive cancer).

### SUMMARY OF THE DISCLOSURE

The present disclosure relates to agonistic ICOS-binding molecules comprising at least one antigen binding domain capable of specific binding to a tumor-associated antigen and their use in combination with anti-CD3 bispecific antibodies, in particular to their use in a method for treating or delaying progression of cancer. It has been found that the combination therapy described herein is more effective in inducing early T-cell activation, T-cell proliferation, induction of T memory cell and consequently inhibiting tumor growth and eliminating tumor cells compared to treatment with the anti-CD3 bispecific antibodies alone. In particular, the agonistic ICOS-binding molecule comprising at least one antigen binding domain capable of specific binding to a tumor-associated antigen is a bispecific agonistic ICOS-binding molecule comprising at least one antigen binding domain capable of specific binding to a tumor-associated antigen.

In one aspect, the invention provides an agonistic ICOS-binding molecule comprising at least one antigen binding domain capable of specific binding to a tumor-associated antigen for use in a method for treating or delaying progression of cancer, wherein the agonistic ICOS-binding molecule comprising at least one antigen binding domain capable of specific binding to a tumor-associated antigen is used in combination with a T-cell activating anti-CD3 bispecific antibody specific for a tumor-associated antigen. In one aspect, the T-cell activating anti-CD3 bispecific antibody specific for a tumor-associated antigen is an anti-CEA/anti-CD3 bispecific antibody.

In one aspect, the invention provides a bispecific agonistic ICOS-binding molecule comprising at least one antigen binding domain capable of specific binding to a tumor-associated antigen for use in a method for treating or delaying progression of cancer, wherein the agonistic ICOS-binding molecule comprises at least one antigen binding domain capable of specific binding to a tumor-associated antigen and i) at least one ICOS-L comprising the amino acid sequence of SEQ ID NO: 1 or SEQ ID NO:2; or ii) one antigen binding domain that is capable of agonistic binding to human ICOS comprising the amino acid sequence of SEQ ID NO:3; and is used in combination with a T-cell activating anti-CD3 bispecific antibody specific for a tumor-associated antigen. In one aspect, the T-cell activating anti-CD3 bispecific antibody binds to the same tumor-associated antigen as the bispecific agonistic ICOS-binding molecule. In another aspect, the T-cell activating anti-CD3 bispecific antibody binds to a tumor-associated antigen different from the bispecific agonistic ICOS-binding molecule. In one aspect, the T-cell activating anti-CD3 bispecific antibody specific for a tumor-associated antigen is an anti-CEA/anti-CD3 bispecific antibody. In a further aspect, provided is an agonistic ICOS-binding molecule comprising at least one antigen binding domain capable of specific binding to a tumor-associated antigen for use in a method for treating or delaying progression of cancer, wherein the agonistic ICOS-binding molecule comprising at least one antigen binding domain capable of specific binding to a tumor-associated antigen and the T-cell activating anti-CD3 bispecific antibody are administered together in a single composition or administered separately in two or more different compositions. In one aspect, they are administered separately in two different compositions. In a particular aspect, the agonistic ICOS-binding molecule comprising at least one antigen binding domain capable of specific binding to a tumor-associated antigen acts synergistically with the T-cell activating anti-CD3 bispecific antibody.

In one aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen comprises at least one ICOS-L or fragments thereof. In one aspect, the agonistic ICOS-binding molecule comprising at least one antigen binding domain capable of specific binding to a tumor-associated antigen comprises two ICOS-L or fragments thereof. In a particular aspect, the agonistic ICOS-binding molecule comprising at least one antigen binding domain capable of specific binding to a tumor-associated antigen comprises at least one ICOS-L comprising the amino acid sequence of SEQ ID NO: 1 or SEQ ID NO:2.

In a further aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprises at least one antigen binding domain that is capable of agonistic binding to ICOS. In particular, the agonistic ICOS-binding molecule comprising at least one antigen binding domain capable of specific binding to a tumor-associated antigen comprises at least one antigen binding domain that is capable of agonistic binding to human ICOS comprising the amino acid sequence of SEQ ID NO:3.

In another aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen is an agonistic ICOS-binding molecule comprising at least one antigen binding domain capable of specific binding to Fibroblast activation protein (FAP). In one aspect, the agonistic ICOS-binding molecule comprises at least one antigen binding domain capable of specific binding to FAP comprising (a) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or (b) a heavy chain variable region (V_{H}FAP) comprising (i) CDR-H1 comprising the amino acid sequence of SEQ ID NO: 12, (ii) CDR-H2 comprising the amino acid sequence of SEQ ID NO: 13, and (iii) CDR-H3 comprising the amino acid sequence of SEQ ID NO: 14, and a light chain variable region (V_{L}FAP) comprising (iv) CDR-L1 comprising the amino acid sequence of SEQ ID NO: 15, (v) CDR-L2 comprising the amino acid sequence of SEQ ID NO: 16, and (vi) CDR-L3 comprising the amino acid sequence of SEQ ID NO: 17. In a particular aspect, the agonistic ICOS-binding molecule comprises at least one antigen binding domain capable of specific binding to FAP comprising a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9.

In one aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11 or wherein the antigen binding domain capable of specific binding to FAP comprises a heavy chain variable region (V_{H}FAP) comprising an amino acid sequence of SEQ ID NO: 18 and a light chain variable region (V_{L}FAP) comprising an amino acid sequence of SEQ ID NO: 19. Particularly, the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11.

In another aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen is an agonistic ICOS-binding molecule comprising at least one antigen binding domain capable of specific binding to Carcinoembroynic antigen (CEA). In one aspect, the agonistic ICOS-binding molecule comprises at least one antigen binding domain capable of specific binding to CEA comprising (a) a heavy chain variable region (V_{H}CEA) comprising (i) CDR-H1 comprising the amino acid sequence of SEQ ID NO: 145, (ii) CDR-H2 comprising the amino acid sequence of SEQ ID NO: 146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO: 148, (v) CDR-L2 comprising the amino acid sequence of SEQ ID NO: 149, and (vi) CDR-L3 comprising the amino acid sequence of SEQ ID NO: 150, or (b) a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163. In a particular aspect, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO:145, (ii) CDR-H2 comprising the amino acid sequence of SEQ ID NO:146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO: 148, (v) CDR-L2 comprising the amino acid sequence of SEQ ID NO: 149, and (vi) CDR-L3 comprising the amino acid sequence of SEQ ID NO: 150. In a further aspect, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163.

In one aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprises at least one antigen binding domain capable of specific binding to CEA comprising a heavy chain variable region (V_{H}CEA) comprising an amino acid sequence of SEQ ID NO: 151 and a light chain variable region (V_{L}CEA) comprising an amino acid sequence of SEQ ID NO: 152 or wherein the antigen binding domain capable of specific binding to CEA comprises a heavy chain variable region (V_{H}CEA) comprising an amino acid sequence of SEQ ID NO: 164 and a light chain variable region (V_{L}CEA) comprising an amino acid sequence of SEQ ID NO: 165. Particularly, the agonistic ICOS-binding molecule comprises at least one antigen binding domain capable of specific binding to CEA comprising a heavy chain variable region (V_{H}CEA) comprising an amino acid sequence of SEQ ID NO: 151 and a light chain variable region (V_{L}CEA) comprising an amino acid sequence of SEQ ID NO: 152. In another aspect, the agonistic ICOS-binding molecule comprises at least one antigen binding domain capable of specific binding to FAP comprising a heavy chain variable region (V_{H}CEA) comprising an amino acid sequence of SEQ ID NO: 164 and a light chain variable region (V_{L}CEA) comprising an amino acid sequence of SEQ ID NO: 165.

In another aspect, provided is an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25. In a particular aspect, the agonistic ICOS-binding molecule comprising at least one antigen binding domain that binds to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27.

In a further aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen comprises an IgG Fc domain, specifically an IgG1 Fc domain or an IgG4 Fc domain. In a particular aspect, the IgG Fc domain is an IgG1 Fc domain. In yet another particular aspect, the agonistic ICOS-binding molecule comprising at least one antigen binding domain capable of specific binding to a tumor-associated antigen comprises a Fc domain that comprises one or more amino acid substitution that reduces binding to an Fc receptor and/or effector function. More particularly, the agonistic ICOS-binding molecule comprising at least one antigen binding domain capable of specific binding to a tumor-associated antigen comprises an IgG1 Fc domain comprising the amino acid substitutions L234A, L235A and P329G (Kabat EU numbering).

In one aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprises a first heavy chain comprising an amino acid sequence of SEQ ID NO:28, a first light chain comprising an amino acid sequence of SEQ ID NO:29, a second heavy chain comprising an amino acid sequence of SEQ ID NO:30, and a second light chain comprising an amino acid sequence of SEQ ID NO:31. In another aspect, the agonistic ICOS-binding molecule comprises a first heavy chain comprising an amino acid sequence of SEQ ID NO:32, a second heavy chain comprising an amino acid sequence of SEQ ID NO:66, and one light chain comprising the amino acid sequence of SEQ ID NO:29. In another aspect, there is provided an agonistic ICOS-binding molecule comprising at least one antigen binding domain capable of specific binding to a tumor-associated antigen for use in a method for treating or delaying progression of cancer, wherein the agonistic ICOS-binding molecule comprises monovalent binding to a tumor associated target and bivalent binding to ICOS. In one aspect, the agonistic ICOS-binding molecule comprises a first heavy chain comprising an amino acid sequence of SEQ ID NO:32, a second heavy chain comprising an amino acid sequence of SEQ ID NO:33, and a two light chains comprising an amino acid sequence of SEQ ID NO:29.

In a further aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprises a first heavy chain comprising an amino acid sequence of SEQ ID NO: 155, a first light chain comprising an amino acid sequence of SEQ ID NO:29, a second heavy chain comprising an amino acid sequence of SEQ ID NO: 156, and a second light chain comprising an amino acid sequence of SEQ ID NO: 157. In a further aspect, the agonistic ICOS-binding molecule comprises a first heavy chain comprising an amino acid sequence of SEQ ID NO: 137, a first light chain comprising an amino acid sequence of SEQ ID NO:131, a second heavy chain comprising an amino acid sequence of SEQ ID NO:168, and a second light chain comprising an amino acid sequence of SEQ ID NO: 169. In another aspect, there is provided an agonistic ICOS-binding molecule comprising at least one antigen binding domain capable of specific binding to a tumor-associated antigen for use in a method for treating or delaying progression of cancer, wherein the agonistic ICOS-binding molecule comprises monovalent binding to a tumor associated target and bivalent binding to ICOS. In one aspect, the agonistic ICOS-binding molecule comprises a first heavy chain comprising an amino acid sequence of SEQ ID NO: 153, a second heavy chain comprising an amino acid sequence of SEQ ID NO: 154, and a two light chains comprising an amino acid sequence of SEQ ID NO:29. In another aspect, the agonistic ICOS-binding molecule comprises a first heavy chain comprising an amino acid sequence of SEQ ID NO: 166, a second heavy chain comprising an amino acid sequence of SEQ ID NO: 167, and a two light chains comprising an amino acid sequence of SEQ ID NO:131.

In another aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the T-cell activating anti-CD3 bispecific antibody is an anti-CEA/anti-CD3 bispecific antibody. In one aspect, the T-cell activating anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA). In particular, the T-cell activating anti-CD3 bispecific antibody comprises a first antigen binding domain comprising a heavy chain variable region (V_{H}CD3) comprising (i) CDR-H1 comprising the amino acid sequence of SEQ ID NO:34, (ii) CDR-H2 comprising the amino acid sequence of SEQ ID NO:35, and (iii) CDR-H3 comprising the amino acid sequence of SEQ ID NO:36, and a light chain variable region (V_{L}CD3) comprising (iv) CDR-L1 comprising the amino acid sequence of SEQ ID NO:37, (v) CDR-L2 comprising the amino acid sequence of SEQ ID NO:38, and (vi) CDR-L3 comprising the amino acid sequence of SEQ ID NO:39. More particularly, the T-cell activating anti-CD3 bispecific antibody comprises a first antigen binding domain comprising a heavy chain variable region (V_{H}CD3) comprising the amino acid sequence of SEQ ID NO:40 and/or a light chain variable region (V_{L}CD3) comprising the amino acid sequence of SEQ ID NO:41.

In a further aspect, the T-cell activating anti-CD3 bispecific antibody as defined herein before comprises a second antigen binding domain comprising (a) a heavy chain variable region (V_{H}CEA) comprising (i) CDR-H1 comprising the amino acid sequence of SEQ ID NO:42, (ii) CDR-H2 comprising the amino acid sequence of SEQ ID NO:43, and (iii) CDR-H3 comprising the amino acid sequence of SEQ ID NO:44, and a light chain variable region (V_{L}CEA) comprising (iv) CDR-L1 comprising the amino acid sequence of SEQ ID NO:45, (v) CDR-L2 comprising the amino acid sequence of SEQ ID NO:46, and (vi) CDR-L3 comprising the amino acid sequence of SEQ ID NO:47, or (b) a heavy chain variable region (V_{H}CEA) comprising (i) CDR-H1 comprising the amino acid sequence of SEQ ID NO:50, (ii) CDR-H2 comprising the amino acid sequence of SEQ ID NO:51, and (iii) CDR-H3 comprising the amino acid sequence of SEQ ID NO:52, and a light chain variable region (V_{L}CEA) comprising (iv) CDR-L1 comprising the amino acid sequence of SEQ ID NO:53, (v) CDR-L2 comprising the amino acid sequence of SEQ ID NO:54, and (vi) CDR-L3 comprising the amino acid sequence of SEQ ID NO:55. In one aspect, the T-cell activating anti-CD3 bispecific antibody comprises a second antigen binding domain comprising a heavy chain variable region (V_{H}CEA) comprising the amino acid sequence of SEQ ID NO:48 and/or a light chain variable region (V_{L}CEA) comprising the amino acid sequence of SEQ ID NO:49 or a second antigen binding domain comprising a heavy chain variable region (V_{H}CEA) comprising the amino acid sequence of SEQ ID NO:56 and/or a light chain variable region (V_{L}CEA) comprising the amino acid sequence of SEQ ID NO:57.

In another aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the T-cell activating anti-CD3 bispecific antibody is an anti-CEA/anti-CD3 bispecific antibody and wherein the anti-CEA/anti-CD3 bispecific antibody comprises a third antigen binding domain that binds to CEA. In particular, the third antigen binding domain comprises (a) a heavy chain variable region (V_{H}CEA) comprising (i) CDR-H1 comprising the amino acid sequence of SEQ ID NO:42, (ii) CDR-H2 comprising the amino acid sequence of SEQ ID NO:43, and (iii) CDR-H3 comprising the amino acid sequence of SEQ ID NO:44, and a light chain variable region (V_{L}CEA) comprising (iv) CDR-L1 comprising the amino acid sequence of SEQ ID NO:45, (v) CDR-L2 comprising the amino acid sequence of SEQ ID NO:46, and (vi) CDR-L3 comprising the amino acid sequence of SEQ ID NO:47, or (b) a heavy chain variable region (V_{H}CEA) comprising (i) CDR-H1 comprising the amino acid sequence of SEQ ID NO:50, (ii) CDR-H2 comprising the amino acid sequence of SEQ ID NO:51, and (iii) CDR-H3 comprising the amino acid sequence of SEQ ID NO:52, and a light chain variable region (V_{L}CEA) comprising (iv) CDR-L1 comprising the amino acid sequence of SEQ ID NO:53, (v) CDR-L2 comprising the amino acid sequence of SEQ ID NO:54, and (vi) CDR-L3 comprising the amino acid sequence of SEQ ID NO:55. More particularly, the third antigen binding domain comprises a heavy chain variable region (V_{H}CEA) comprising the amino acid sequence of SEQ ID NO:48 and/or a light chain variable region (V_{L}CEA) comprising the amino acid sequence of SEQ ID NO:49 or a heavy chain variable region (V_{H}CEA) comprising the amino acid sequence of SEQ ID NO:56 and/or a light chain variable region (V_{L}CEA) comprising the amino acid sequence of SEQ ID NO:57.

In a further aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the T-cell activating anti-CD3 bispecific antibody specific for a tumor-associated antigen comprises an IgG Fc domain, specifically an IgG1 Fc domain or an IgG4 Fc domain. In a particular aspect, the IgG Fc domain is an IgG1 Fc domain. In yet another particular aspect, the T-cell activating anti-CD3 bispecific antibody specific for a tumor-associated antigen comprises a Fc domain that comprises one or more amino acid substitution that reduces binding to an Fc receptor and/or effector function. More particularly, the T-cell activating anti-CD3 bispecific antibody specific for a tumor-associated antigen comprises an IgG1 Fc domain comprising the amino acid substitutions L234A, L235A and P329G (Kabat EU numbering).

In a particular aspect, there is provided an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the T-cell activating anti-CD3 bispecific antibody comprises (a) the amino acid sequences of SEQ ID NO:58, SEQ ID NO:59, SEQ ID NO:60 and SEQ ID NO:61, or (b) the amino acid sequences of SEQ ID NO:62, SEQ ID NO:63, SEQ ID NO:64 and SEQ ID NO:65. More particularly, the T-cell activating anti-CD3 bispecific antibody comprises the amino acid sequences of SEQ ID NO:58, SEQ ID NO:59, SEQ ID NO:60 and SEQ ID NO:61.

In another aspect, the invention provides an agonistic ICOS-binding molecule comprising at least one antigen binding domain capable of specific binding to a tumor-associated antigen for use in combination with a T-cell activating anti-CD3 bispecific antibody specific for a tumor-associated antigen in a method for treating or delaying progression of cancer, wherein the agonistic ICOS-binding molecule comprising at least one antigen binding domain capable of specific binding to a tumor-associated antigen is used in combination with a T-cell activating anti-CD3 bispecific antibody specific for a tumor-associated antigen and in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

In a further aspect, the invention provides a pharmaceutical product comprising (A) a first composition comprising as active ingredient an agonistic ICOS-binding molecule comprising at least one antigen binding domain capable of specific binding to a tumor-associated antigen and a pharmaceutically acceptable excipient; and (B) a second composition comprising a T-cell activating anti-CD3 bispecific antibody specific for a tumor-associated antigen and a pharmaceutically acceptable excipient, for use in the combined, sequential or simultaneous, treatment of a disease, in particular cancer. In particular, the agonistic ICOS-binding molecule comprising at least one antigen binding domain capable of specific binding to a tumor-associated antigen is a molecule as defined herein before. In another aspect, the T-cell activating anti-CD3 bispecific antibody specific for a tumor-associated antigen is an anti-CD3 bispecific antibody as defined herein before.

In yet another aspect, the invention relates to a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients. In a particular aspect, there is provided a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients for use in the treatment of cancer, more particularly for the treatment of solid tumors.

In another aspect, provided is a method for treating or delaying progression of cancer by administering a bispecific agonistic ICOS-binding tumor-targeted molecule in combination with a T-cell activating anti-CD3 bispecific antibody, wherein the bispecific agonistic ICOS-binding molecule comprises at least one antigen binding domain capable of specific binding to a tumor-associated antigen; and i) at least one ICOS-L comprising the amino acid sequence of SEQ ID NO: 1 or SEQ ID NO:2; or ii) one antigen binding domain that is capable of agonistic binding to human ICOS comprising the amino acid sequence of SEQ ID NO:3; wherein the T-cell activating anti-CD3 bispecific antibody binds to a tumor-associated antigen. In one aspect, the T-cell activating anti-CD3 bispecific antibody binds to the same tumor-associated antigen as the bispecific agonistic ICOS-binding molecule. In another aspect, the T-cell activating anti-CD3 bispecific antibody binds to a tumor-associated antigen different from the bispecific agonistic ICOS-binding molecule.

In another aspect, the invention provides an agonistic ICOS-binding molecule comprising at least one antigen binding domain capable of specific binding to a tumor-associated antigen, wherein the tumor-associated antigen is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2.

In one aspect, there is provided an agonistic ICOS-binding molecule comprising at least one antigen binding domain capable of specific binding to a tumor-associated antigen, wherein the tumor-associated antigen is FAP. In particular, the agonistic ICOS-binding molecule comprises at least one antigen binding domain capable of specific binding to FAP comprising (a) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or (b) a heavy chain variable region (V_{H}FAP) comprising (i) CDR-H1 comprising the amino acid sequence of SEQ ID NO: 12, (ii) CDR-H2 comprising the amino acid sequence of SEQ ID NO: 13, and (iii) CDR-H3 comprising the amino acid sequence of SEQ ID NO: 14, and a light chain variable region (V_{L}FAP) comprising (iv) CDR-L1 comprising the amino acid sequence of SEQ ID NO: 15, (v) CDR-L2 comprising the amino acid sequence of SEQ ID NO: 16, and (vi) CDR-L3 comprising the amino acid sequence of SEQ ID NO: 17. More particularly, the agonistic ICOS-binding molecule comprises at least one antigen binding domain capable of specific binding to FAP comprising a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9. In one aspect, the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11 or at least one antigen binding domain capable of specific binding to FAP comprising a heavy chain variable region (V_{H}FAP) comprising an amino acid sequence of SEQ ID NO: 18 and a light chain variable region (V_{L}FAP) comprising an amino acid sequence of SEQ ID NO: 19. In a further aspect, the agonistic ICOS-binding molecule comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25. More particularly, the agonistic ICOS-binding molecule comprising at least one antigen binding domain capable of specific binding to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27. In a further aspect, the agonistic ICOS-binding molecule comprising at least one antigen binding domain capable of specific binding to a tumor-associated antigen comprises an IgG Fc domain, specifically an IgG1 Fc domain or an IgG4 Fc domain. More particularly, the agonistic ICOS-binding molecule comprising at least one antigen binding domain capable of specific binding to a tumor-associated antigen comprises an IgG1 Fc domain. In a particular aspect, the agonistic ICOS-binding molecule comprising at least one antigen binding domain capable of specific binding to a tumor-associated antigen comprises a Fc domain that comprises one or more amino acid substitution that reduces binding to an Fc receptor and/or effector function. More particularly, the agonistic ICOS-binding molecule comprising at least one antigen binding domain capable of specific binding to a tumor-associated antigen comprises an IgG1 Fc domain comprising the amino acid substitutions L234A, L235A and P329G (Kabat EU numbering).

In one further aspect, there is provided an agonistic ICOS-binding molecule comprising at least one antigen binding domain capable of specific binding to a tumor-associated antigen, wherein the tumor-associated antigen is CEA. In particular, the agonistic ICOS-binding molecule comprises at least one antigen binding domain capable of specific binding to CEA (a) a heavy chain variable region (V_{H}CEA) comprising (i) CDR-H1 comprising the amino acid sequence of SEQ ID NO: 145, (ii) CDR-H2 comprising the amino acid sequence of SEQ ID NO: 146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO:148, (v) CDR-L2 comprising the amino acid sequence of SEQ ID NO:149, and (vi) CDR-L3 comprising the amino acid sequence of SEQ ID NO: 150, or (b) a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163. More particularly, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO: 145, (ii) CDR-H2 comprising the amino acid sequence of SEQ ID NO: 146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO: 148, (v) CDR-L2 comprising the amino acid sequence of SEQ ID NO: 149, and (vi) CDR-L3 comprising the amino acid sequence of SEQ ID NO: 150. In one further aspect, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163. In one aspect, the agonistic ICOS-binding molecule comprises at least one antigen binding domain capable of specific binding to CEA comprising a heavy chain variable region (V_{H}CEA) comprising an amino acid sequence of SEQ ID NO: 151 and a light chain variable region (V_{L}CEA) comprising an amino acid sequence of SEQ ID NO: 152 or the antigen binding domain capable of specific binding to CEA comprises a heavy chain variable region (V_{H}CEA) comprising an amino acid sequence of SEQ ID NO: 164 and a light chain variable region (V_{L}CEA) comprising an amino acid sequence of SEQ ID NO: 165. In a further aspect, the agonistic ICOS-binding molecule comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25. More particularly, the agonistic ICOS-binding molecule comprising at least one antigen binding domain capable of specific binding to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27. In another aspect, the agonistic ICOS-binding molecule comprises at least one antigen binding domain capable of specific binding to ICOS comprising a heavy chain variable region (V_{H}ICOS) comprising (i) CDR-H1 comprising the amino acid sequence of SEQ ID NO: 123, (ii) CDR-H2 comprising the amino acid sequence of SEQ ID NO: 124, and (iii) CDR-H3 comprising the amino acid sequence of SEQ ID NO: 125, and a light chain variable region (V_{L}ICOS) comprising (iv) CDR-L1 comprising the amino acid sequence of SEQ ID NO: 126, (v) CDR-L2 comprising the amino acid sequence of SEQ ID NO: 127, and (vi) CDR-L3 comprising the amino acid sequence of SEQ ID NO: 128. More particularly, the agonistic ICOS-binding molecule comprising at least one antigen binding domain capable of specific binding to a tumor-associated antigen comprises at least one antigen binding domain comprising a heavy chain variable region (V_{H}ICOS) comprising an amino acid sequence of SEQ ID NO: 129 and a light chain variable region (V_{L}ICOS) comprising an amino acid sequence of SEQ ID NO: 130. In a further aspect, the agonistic ICOS-binding molecule comprising at least one antigen binding domain that binds to a tumor-associated antigen comprises an IgG Fc domain, specifically an IgG1 Fc domain or an IgG4 Fc domain. More particularly, the agonistic ICOS-binding molecule comprising at least one antigen binding domain that binds to a tumor-associated antigen comprises an IgG1 Fc domain. In a particular aspect, the agonistic ICOS-binding molecule comprising at least one antigen binding domain capable of specific binding to a tumor-associated antigen comprises a Fc domain that comprises one or more amino acid substitution that reduces binding to an Fc receptor and/or effector function. More particularly, the agonistic ICOS-binding molecule comprising at least one antigen binding domain capable of specific binding to a tumor-associated antigen comprises an IgG1 Fc domain comprising the amino acid substitutions L234A, L235A and P329G (Kabat EU numbering).

In one aspect, the invention provides an agonistic ICOS-binding molecule comprising at least one antigen binding domain capable of specific binding to a tumor-associated antigen, wherein the agonistic ICOS-binding molecule comprises a first heavy chain comprising an amino acid sequence of SEQ ID NO:28, a first light chain comprising an amino acid sequence of SEQ ID NO:29, a second heavy chain comprising an amino acid sequence of SEQ ID NO:30, and a second light chain comprising an amino acid sequence of SEQ ID NO:31. In a further aspect, the agonistic ICOS-binding molecule comprises a first heavy chain comprising an amino acid sequence of SEQ ID NO:32, a second heavy chain comprising an amino acid sequence of SEQ ID NO:66, and a light chain comprising an amino acid sequence of SEQ ID NO:29. In another aspect, there is provided an agonistic ICOS-binding molecule comprising at least one antigen binding domain capable of specific binding to a tumor-associated antigen, wherein the agonistic ICOS-binding molecule comprises monovalent binding to a tumor associated target and bivalent binding to ICOS. In one aspect, the agonistic ICOS-binding molecule comprises a first heavy chain comprising an amino acid sequence of SEQ ID NO:32, a second heavy chain comprising an amino acid sequence of SEQ ID NO:33, and a two light chains comprising an amino acid sequence of SEQ ID NO:29.

In one further aspect, provided is an agonistic ICOS-binding molecule comprising at least one antigen binding domain capable of specific binding to a tumor-associated antigen, wherein the agonistic ICOS-binding molecule comprises a first heavy chain comprising an amino acid sequence of SEQ ID NO: 155, a first light chain comprising an amino acid sequence of SEQ ID NO:29, a second heavy chain comprising an amino acid sequence of SEQ ID NO: 156, and a second light chain comprising an amino acid sequence of SEQ ID NO: 157. In a further aspect, the agonistic ICOS-binding molecule comprises a first heavy chain comprising an amino acid sequence of SEQ ID NO: 137, a first light chain comprising an amino acid sequence of SEQ ID NO:131, a second heavy chain comprising an amino acid sequence of SEQ ID NO:168, and a second light chain comprising an amino acid sequence of SEQ ID NO: 169. In another aspect, there is provided an agonistic ICOS-binding molecule comprising at least one antigen binding domain capable of specific binding to a tumor-associated antigen, wherein the agonistic ICOS-binding molecule comprises monovalent binding to a tumor associated target and bivalent binding to ICOS. In one aspect, the agonistic ICOS-binding molecule comprises a first heavy chain comprising an amino acid sequence of SEQ ID NO: 153, a second heavy chain comprising an amino acid sequence of SEQ ID NO: 154, and a two light chains comprising an amino acid sequence of SEQ ID NO:29. In one further aspect, the agonistic ICOS-binding molecule comprises a first heavy chain comprising an amino acid sequence of SEQ ID NO: 166, a second heavy chain comprising an amino acid sequence of SEQ ID NO: 167, and a two light chains comprising an amino acid sequence of SEQ ID NO:131.

According to another aspect of the invention, there is provided an isolated polynucleotide encoding an agonistic ICOS-binding molecule comprising at least one antigen binding domain capable of specific binding to a tumor-associated antigen as described herein before. The invention further provides a vector, particularly an expression vector, comprising the isolated polynucleotide of the invention and a host cell comprising the isolated polynucleotide or the vector of the invention. In some aspects the host cell is a eukaryotic cell, particularly a mammalian cell.

In another aspect, provided is a method for producing an agonistic ICOS-binding molecule comprising at least one antigen binding domain capable of specific binding to a tumor-associated antigen as described herein before, comprising the steps of (i) culturing the host cell of the invention under conditions suitable for expression of the antigen binding molecule, and (ii) recovering the antigen binding molecule. The invention also encompasses the agonistic ICOS-binding molecule comprising at least one antigen binding domain capable of specific binding to a tumor-associated antigen as described herein produced by the method of the invention.

The invention further provides a pharmaceutical composition comprising an agonistic ICOS-binding molecule comprising at least one antigen binding domain capable of specific binding to a tumor-associated antigen as described herein before and at least one pharmaceutically acceptable excipient.

Also encompassed by the invention is the agonistic ICOS-binding molecule comprising at least one antigen binding domain capable of specific binding to a tumor-associated antigen as described herein before, or the pharmaceutical composition comprising the agonistic ICOS-binding molecule comprising at least one antigen binding domain capable of specific binding to a tumor-associated antigen, for use as a medicament.

In one aspect, provided is the agonistic ICOS-binding molecule comprising at least one antigen binding domain capable of specific binding to a tumor-associated antigen as described herein before or the pharmaceutical composition comprising the agonistic ICOS-binding molecule comprising at least one antigen binding domain capable of specific binding to a tumor-associated antigen, for use
(i) in stimulating T cell response,
(ii) in supporting survival of activated T cells,
(iii) in the treatment of infections,
(iv) in the treatment of cancer,
(v) in delaying progression of cancer, or
(vi) in prolonging the survival of a patient suffering from cancer.

In a specific aspect, there is provided the agonistic ICOS-binding molecule comprising at least one antigen binding domain that binds to a tumor-associated antigen as described herein before, or the pharmaceutical composition comprising the agonistic ICOS-binding molecule comprising at least one antigen binding domain capable of specific binding to a tumor-associated antigen as described herein before, for use in the treatment of cancer.

In another specific aspect, the invention provides the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain capable of specific binding to a tumor-associated antigen is for administration in combination with a chemotherapeutic agent, radiation and/or other agents for use in cancer immunotherapy.

In a further aspect, provided is the agonistic ICOS-binding molecule comprising at least one antigen binding domain capable of specific binding to a tumor-associated antigen as described herein before for use in the treatment of cancer, wherein the agonistic ICOS-binding molecule comprising at least one antigen binding domain capable of specific binding to a tumor-associated antigen is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

In a further aspect, the invention provides a method of inhibiting the growth of tumor cells in an individual comprising administering to the individual an effective amount of the agonistic ICOS-binding molecule comprising at least one antigen binding domain that binds to a tumor-associated antigen as described herein before, or the pharmaceutical composition comprising the agonistic ICOS-binding molecule comprising at least one antigen binding domain that binds to a tumor-associated antigen as described herein before, to inhibit the growth of the tumor cells.

Also provided is the use of the agonistic ICOS-binding molecule comprising at least one antigen binding domain capable of specific binding to a tumor-associated antigen as described herein before for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the agonistic ICOS-binding molecule comprising at least one antigen binding domain that binds to a tumor-associated antigen as described herein before in a pharmaceutically acceptable form. In a specific aspect, the disease is cancer. In any of the above aspects the individual is a mammal, particularly a human.

### BRIEF DESCRIPTION OF THE DRAWINGS

FIG. 1A-E: Schematic Figures of all FAP- or CEA-targeted ICOS molecules & their untargeted (DP47) reference molecules. In FIG. 1A and FIG. 1B different types of targeted ICOS bispecific antibodies in 1+1 format are shown. FIG. 1A includes either FAP- or CEA as tumor-associated targeting moiety. A FAP- or CEA-ICOS antibody in 2+1 format (monovalent for the tumor-associated target) is shown in FIG. 1C and in FIGS. 1D and 1E untargeted (DP47)-ICOS bispecific antibodies in 1+1 format (FIG. 1D) and 2+1 format (FIG. 1E) are shown.

FIG. 1F: Schematic Figure of a human T-cell activating anti-CD3 bispecific antibody specific for a tumor-associated antigen (CEA TCB).

FIGS. 2A and 2B: Schematic Figure of a FAP-targeted mICOS-L molecule (FIG. 2A) and an untargeted (DP47)-mICOS-L reference molecule (FIG. 2B).

FIG. 2C: Schematic Figure of a murine T-cell activating anti-CD3 bispecific antibody specific for a tumor-associated antigen (CEA TCB).

FIGS. 3A-D: ICOS expression on T-cells from healthy donors, TILs or on T-cells isolated from normal tissue of the same patient.

FIGS. 3E-3H: ICOS expression on TILs versus human PBMCs at baseline versus control without versus with TCB treatment in the presence of CEA-positive Lovo target cells. Median fluorescence intensities and percent of ICOS-positive T-cells, as determined by flow cytometry. Depicted are technical triplicates with SD. Isotypes were substracted.

FIGS. 4A-4D: Increase of human ICOS expression upon CEACAM5-TCB-mediated activation of human T cells, as determined by flow cytometry. FIGS. 4A and 4B show the dose-dependent increase in ICOS⁺ CD4 (FIG. 4A) and CD8⁺ (FIG. 4B) T-Cells upon incubation with CEACAM5 TCB for 48 hours. The graphs show technical triplicates, error bars indicate SD. FIGS. 4C and 4D show the increase in percentage of ICOS⁺ CD4⁺ (FIG. 4C) and CD8⁺ (FIG. 4D) T Cells upon CEA TCB (100 nM) or CEACAM5 TCB (20 nM) treatment for 48 h for 5 different healthy human PBMC donors.

FIGS. 5A-5F: Increase of murine ICOS expression on murine CD4⁺, Treg or CD8⁺ T-cells upon mTCB-mediated activation of murine T cells. Percentage (FIGS. 5A-5C) and Median Fluorescence Intensities (MFI, FIGS. 5D-5F) of ICOS-positive cells of either murine CD4 (FIGS. 5A and 5D), Treg (FIGS. 5B and 5E) or CD8 T cells (FIGS. 5C and 5F) after 48 h of co-incubation of MC38-hCEA tumor target cells, murine splenocytes from hCEA(HO) Tg mice and increasing concentrations of mCEA-TCB. The graphs show technical triplicates, error bars indicate SD, isotype values have been substracted.

FIGS. 6A-6C: Binding of different FAP-ICOS molecules to human ICOS (FIGS. 6A and 6B) and human FAP (FIG. 6C) on cells, as determined by flow cytometry. Median fluorescence intensities for binding of different FAP- or DP47-ICOS molecules to activated PBMCs (Using Gibco #11161D, 48 hours, 1:2 Bead to Cell Ratio), respective to human FAP-expressing NIH/3T3-hFAP cells, as measured by flow cytometry. Depicted are technical triplicates with SD.

FIGS. 7A and 7B: Binding of FAP-mICOS-L to murine ICOS (FIG. 7A)- and murine FAP (FIG. 7B)-expressing cells, as determined by flow cytometry. Median fluorescence intensities for binding of FAP-mICOS-L to stable CHO-K1 transfectants, over-expressing murine ICOS (A), respective to 3T3-mFAP cells (parental cell line ATCC # CCL-92, modified to stably overexpress murine FAP (B), as measured by flow cytometry. Depicted are technical triplicates with SD.

FIGS. 8A-8I: Evaluation of different FAP-versus untargeted ICOS molecules. In FIG. 8A, potential superagonism was addressed, using a Jurkat-NFAT reporter assay. The graphs show luminescence signal intensities, i.e. luminescence intensities of preactivated Jurkat-NFAT-Luc2P cells (Promega # CS 176501) with different plate-bound aICOS constructs in presence or absence of simultaneously coated aCD3. Depicted are technical triplicates with SEM. In FIGS. 8B to 8E, the impact of crosslinking of ICOS molecules via FAP, as assessed in a T-cell activation assay by flow cytometry is shown. Depicted are surface expression levels of early T-cell activation CD69 and late activation marker CD25 on CD4⁺ T cells (FIGS. 8B and 8C) or CD8⁺ T-cells (FIGS. 8D and 8E) as Median Fluorescence Intensities (MFI) or as Area under the curve AUC (FIGS. 8F to 8I). Median Fluorescence Intensitites (MFI) were measured after 48 h of co-incubation of human PBMC effector, MKN45 tumor cells and 3T3-hFAP fibroblasts at an E:T of 5:1:1 in presence of 80 pM CEACAM5 TCB in presence of increasing concentration of FAP targeted FAP-ICOS constructs or untargeted DP47-ICOS constructs. The graphs show technical triplicates, error bars indicate SD.

FIGS. 9A-9H: Increase of CEACAM5-TCB-mediated T-cell activation by FAP-ICOS bispecific molecules, crosslinked by binding to 3T3-hFAP cells, as determined by flow cytometry. Depicted are expression levels of the CD69 as MFI or percent of positive CD4⁺ and CD8⁺ T-cells in the presence of increasing concentrations of the FAP-ICOS_2+1 molecule (FIGS. 9A-9D) as well as the maximal fold increase of T-cell activation, induced by co-incubation of FAP-ICOS molecules and CEACAM5-TCB for up to 5 different healthy human PBMC donors (FIGS. 9E-9H). Median Fluorescence Intensitites (MFI, FIGS. 9A and 9C) and percentage (FIGS. 9B and 9D) of CD69-positive CD4⁺ T cells (FIGS. 9A and 9B) or CD8⁺ T cells (FIGS. 9C and 9D) are shown after 48 h of co-incubation of human PBMC effector, MKN45 tumor cells and 3T3-hFAP fibroblasts at an E:T of 5:1:1 in presence of 80 pM CEACAM5 TCB in presence of increasing concentration of FAP-ICOS_2+1. The graphs show technical triplicates, error bars indicate SD. FIGS. 9E-9H show the maximal fold increase of T-cell activation, induced by co-incubation of the indicated FAP-ICOS molecules and 80 pM TCB for up to 5 different healthy human PBMC donors.

FIGS. 10A-L: Increased CEACAM5-TCB-mediated T-cell Proliferation and T-Cell Differentiation in absence versus presence of several FAP-ICOS Formats. The graphs show the number of absolute CD4⁺ and CD8⁺ T-cells (FIGS. 10A and 10B), respective numbers of naïve, central memory (Tcm), effector memory (Tem) and CD45RA-positive effector memory (Temra) cells with increasing TCB concentration (FIGS. 10C to 10J) or at a fixed concentration of 2.9 pM (FIGS. 10K and 10L), as determined by flow cytometry. Absolute Cell Counts of CD4⁺ and CD8+(FIGS. 10A and 10B) or CD4 or CD8⁺ Tem, Tcm, Tnaive and Temra Memory T Cell Subsets (FIGS. 10C to 10J) after 96 h of co-incubation of human PBMC effector, MKN45 tumor cells and 3T3-hFAP fibroblasts at an E:T of 5:1:1 in presence of CEACAM5-TCB (increasing concentrations) in presence of 1 nM FAP-ICOS_1+1, FAP-ICOS 2+1., FAP-ICOS_1+1 HT. The graphs show means of technical triplicates, error bars indicate SD. FIGS. 10K and 10L refer to the data set depicted in FIGS. 10C to 10J, highlighting the absolute numbers of naïve, central memory, effector memory or Temra cells at a fixed concentration of 2.9 pM CEACAM5-TCB in absence versus presence of 1 nM of the indicated ICOS molecules. Gating was done as follows: T_{cm}=CD45RO+CCR7−, T_{cm}=CD45RO+CCR7+, Tₑₘᵣₐ=CD45RO−CCR7−, Tₙₐᵢᵥₑ=CD45RO−CCR7+.

FIGS. 11A and 11B: Increased mCEA-TCB-mediated T-cell activation in presence of FAP-mICOS-L. The graphs show percent of CD25 and CD69-positive CD8⁺ and CD4⁺ T-cells, as assessed by flow cytometry. Percentage of CD25- and CD69-positive CD8⁺ T cells (FIG. 11A) or CD4⁺ T cells (FIG. 11B) after 48 h of co-incubation of MC38-hCEA tumor cells, 3T3-mFAP fibroblasts and murine splenocyte effector cells (C57 Bl/6 mice) at an E:T of 50:1 in presence of 1.5 nM mCEA-TCB in presence or absence of 50 nM FAP-targeted or untargeted reference mICOS-L molecules, as indicated. The graphs show triplicates, error bars indicate SD, isotype values have been subtracted.

FIG. 12: Pharmacokinetic profile of FAP-ICOS_1+1 after single injection in NSG mice.

FIG. 13: Efficacy study with FAP-ICOS_1+1 and CEACAM5-TCB combination in MKN45 Xenograft in humanized mice. Depicted is the study design and the treatment groups.

FIGS. 14A-14E: Efficacy study with FAP-ICOS_1+1 and CEACAM5 TCB combination in MKN45 Xenograft in humanized mice. Shown is the average tumor volume (FIG. 14A) or the growth of tumors in individual mice as plotted on the y-axis (FIGS. 14B to 14D). Tumor weight at day 44 as plotted for individual mice is summarized in FIG. 14E. It can be seen that there is increased TCB-mediated Tumor Regression in the presence of FAP-ICOS_1+1.

FIGS. 15A-15D: Tumor-specific depletion of FoxP3+ Tregs upon combination therapy of FAP-ICOS_1+1 antibody and CEACAM5-TCB in a co-grafting model of MKN45 and 3T3-hFAP cells in humanized NSG mice. Frequency of Treg among CD4⁺ T-cells and ratio of CD8 and Treg cells in spleen (FIGS. 15C and 15D) and tumor (FIGS. 15A and 15B) is shown. Each shape indicates an individual mouse.

FIGS. 16A-16C: Cytokine analysis. Intra-tumoral changes in selected chemokine and cytokine expression upon combination therapy of FAP-ICOS_1+1 antibody and CEACAM5-TCB in a co-grafting model of MKN45 and 3T3-hFAP cells in humanized NSG mice. Each shape indicates an individual mouse. Shown are the data for CCL3 (FIG. 16A), TNF-α (FIG. 16B) and CXCL13 (FIG. 16C).

FIGS. 17A and 17B: Gene expression analysis of remaining tumours of an FAP-ICOS_1+1 combination study with CEACAM5-TCB in a co-grafting model of MKN45 and 3T3-hFAP in humanized NSG mice to identify ICOS-regulated genes. Depicted is the fold increase of significantly upregulated genes TNFAIP6 (p-value is 0.1) and CXCL13 (p value of 0.05), threshold was set to have at least a fold change of 2 as compared to the TCB monotherapy effect. The data for TNFAIP6 are provided in FIG. 17A and the data for CXCL13 are depicted in FIG. 17B.

FIGS. 18A and 18B: Schematic Figures of all FAP- or CEA-targeted anti-murine ICOS molecules. A tumor targeted murine ICOS antibody in 2+1 format (monovalent for the tumor-associated target) is shown in FIG. 18A and in 1+1 format in FIG. 18B.

FIGS. 19A-19D: Binding of CEA-targeted ICOS molecules to ICOS-positive activated human CD4⁺ (upper panel, FIG. 19A) or human CD8⁺ T-cells (lower panel, FIG. 19B), respective CEA-positive MKN45 cells (FIGS. 19C and 19D), as measured by flow cytometry. The graphs show the median fluorescence values (MFI) of technical triplicates, error bars indicate standard deviation (SD).

FIGS. 20A and 20B: Activation of T-cells, depicted as percentage (%) of CD69-positive CD4⁺T-cells after 48 h of co-incubation of human PBMCs, MKN45 and NIH-3T3-hFAP at a cell ratio of 5:1:1 and increasing concentrations of the different ICOS molecules in presence of a 80 pM of the TCB (see FIG. 20A). The graph shows technical triplicates, error bars indicate SD. In FIG. 20B is plotted the fold increase of the percentage (%) of CD69-positive CD4⁺ T-cells induced by the combination of either 200 pM or 1 nM of the ICOS molecules and 80 pM of the CEACAM5-TCB over the CEACAM5-TCB monotherapy.

FIGS. 21A-21C. Binding of anti-murine ICOS molecules to ICOS-positive CHO cells overexpressing murine ICOS (FIG. 21A) or murine NIH-3T3-mFAP cells (FIG. 21B), respective CEA-positive MKN45 cells (FIG. 21C), as measured by flow cytometry. The graphs show the median fluorescence values (MFI) of technical triplicates, error bars indicate standard deviation (SD).

FIGS. 22A-22D: Activation of murine T-cells, depicted as percentage (%) of CD69-positive CD4+(FIG. 22A and FIG. 22C), respective CD8+ T-cells (FIGS. 22B and D) after 48 h of co-incubation of murine splenocytes of C57Bl/6 mice, MC38-hCEA and NIH-3T3-hFAP at a cell ratio of 3:1:1 and increasing concentrations of the different ICOS molecules in presence of 1.5 nM of the murine CEA-TCB. The graph shows technical triplicates, error bars indicate SD.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that capable of specifically binding an antigenic determinant. Examples of antigen binding molecules are antibodies, antibody fragments and scaffold antigen binding proteins.

As used herein, the term “antigen binding domain that binds to a tumor-associated antigen” or “antigen binding domain capable of specific binding to a tumor-associated antigen” or “moiety capable of specific binding to a tumor-associated antigen” refers to a polypeptide molecule that capable of specifically binding to an antigenic determinant. In one aspect, the antigen binding domain is able to activate signaling through its target cell antigen. In a particular aspect, the antigen binding domain is able to direct the entity to which it is attached (e.g. the ICOS agonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “antigen binding domain capable of specific binding to a target cell antigen” refers to the part of the molecule that comprises the area which capable of specifically binding to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a target cell antigen” can also be a Fab fragment or a cross-Fab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites specific for one distinct antigenic determinant in an antigen binding molecule that are specific for one distinct antigenic determinant. As such, the terms “monovalent”, “bivalent”, “tetravalent”, and “hexavalent” denote the presence of one binding site, two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 Daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds.

Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (V_{N}AR fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which capable of specifically binding to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Lilj eblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “tumor-associated antigen” or TAA as used herein refers to an antigenic determinant (different from ICOS) presented on the surface of a target cell, for example a cell in a tumor such as a cancer cell or a cell of the tumor stroma. In certain embodiments, the target cell antigen is an antigen on the surface of a tumor cell. In one embodiment, TAA is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is Fibroblast Activation Protein (FAP) or Carcinoembryonic Antigen (CEA).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:79), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO 80. The amino acid sequence of mouse FAP is shown in UniProt accession no. P97321 (version 126, SEQ ID NO:81), or NCBI RefSeq NP_032012.1. The extracellular domain (ECD) of mouse FAP extends from amino acid position 26 to 761. SEQ ID NO 82 shows the amino acid sequence of a His-tagged mouse FAP ECD. SEQ ID NO 83 the amino acid sequence, of a His-tagged cynomolgus FAP ECD. Preferably, an anti-FAP binding molecule of the invention binds to the extracellular domain of FAP.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:84). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9(2):67-81 (1999)), making it inaccessible to antibody in the blood stream. In contrast to normal tissue, CEA tends to be expressed over the entire surface of cancerous cells (Hammarström S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30 (a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “FolR1” refers to Folate receptor alpha and has been identified as a potential prognostic and therapeutic target in a number of cancers. It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human FolR1 is shown in UniProt accession no. P15328 (SEQ ID NO: 85), murine FolR1 has the amino acid sequence of UniProt accession no. P35846 (SEQ ID NO:86) and cynomolgus FolR1 has the amino acid sequence as shown in UniProt accession no. G7PR14 (SEQ ID NO:87). FolR1 is an N-glycosylated protein expressed on plasma membrane of cells. FolR1 has a high affinity for folic acid and for several reduced folic acid derivatives and mediates delivery of the physiological folate, 5-methyltetrahydrofolate, to the interior of cells. FOLR1 is a desirable target for FOLR1-directed cancer therapy as it is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid. Recent studies have identified that FolR1 expression is particularly high in triple negative breast cancers (Necela et al. PloS One 2015, 10(3), e0127133).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:88). MCSP is a highly glycosylated integral membrane chondroitin sulfate proteoglycan consisting of an N-linked 280 kDa glycoprotein component and a 450-kDa chondroitin sulfate proteoglycan component expressed on the cell membrane (Ross et al., Arch. Biochem. Biophys. 1983, 225:370-38). MCSP is more broadly distributed in a number of normal and transformed cells. In particular, MCSP is found in almost all basal cells of the epidermis. MCSP is differentially expressed in melanoma cells, and was found to be expressed in more than 90% of benign nevi and melanoma lesions analyzed. MCSP has also been found to be expressed in tumors of nonmelanocytic origin, including basal cell carcinoma, various tumors of neural crest origin, and in breast carcinomas.

The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:89). The proto-oncogene “HER2”, (human epidermal growth factor receptor 2) encodes a protein tyrosine kinase (p185HER2) that is related to and somewhat homologous to the human epidermal growth factor receptor. HER2 is also known in the field as c-erbB-2, and sometimes by the name of the rat homolog, neu. Amplification and/or overexpression of HER2 is associated with multiple human malignancies and appears to be integrally involved in progression of 25-30% of human breast and ovarian cancers. Furthermore, the extent of amplification is inversely correlated with the observed median patient survival time (Slamon, D. J. et al., Science 244:707-712 (1989)). The amino acid sequence of human HER2 is shown in UniProt accession no. P04626 (version 230, SEQ ID NO:90). The term “p95HER2” as used herein refers to a carboxy terminal fragment (CTF) of the HER2 receptor protein, which is also known as “611-CTF” or “100-115 kDa p95HER2”. The p95HER2 fragment is generated in the cell through initiation of translation of the HER2 mRNA at codon position 611 of the full-length HER2 molecule (Anido et al, EMBO J 25; 3234-44 (2006)). It has a molecular weight of 100 to 115 kDa and is expressed at the cell membrane, where it can form homodimers maintained by intermolecular disulfide bonds (Pedersen et al., Mol Cell Biol 29, 3319-31 (2009)). An exemplary sequence of human p95HER2 is given in SEQ ID NO: 91.

The term “ICOS” (Inducible T cell COStimulator) refers to any Inducible T cell costimulatory protein from any vertebrate source, including mammals such as primates (e.g. humans) non-human primates (e.g. cynomolgus monkeys) and rodents (e.g. mice and rats), unless otherwise indicated. ICOS, also named AILIM or CD278, is a member of the CD28 superfamily (CD28/CTLA-4 cell-surface receptor family) and is specifically expressed on T cells after initial T cell activation. ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS KO mice demonstrate impaired development of autoimmune phenotypes in a variety of disease models, including diabetes (Th1), airway inflammation (Th2) and EAE neuro-inflammatory models (Th117). In addition to its role in modulating T effector (Teff) cell function, ICOS also modulates T regulatory cells (Tregs). ICOS is expressed at high levels on Tregs, and has been implicated in Treg homeostasis and function. Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival. The term also encompasses naturally occurring variants of ICOS, e.g., splice variants or allelic variants. The amino acid sequence of human ICOS is shown in UniProt (www.uniprot.org) accession no. Q9Y6W8 (SEQ ID NO:3)

As described herein before, ICOS ligand (ICOS-L; B7-H2; B7RP-1; CD275; GL50), also a member of the B7 superfamily, is the membrane bound natural ligand for ICOS and is expressed on the cell surface of B cells, macrophages and dendritic cells. ICOS-L functions as a non-covalently linked homodimer on the cell surface in its interaction with ICOS. Human ICOS-L has also been reported to bind to human CD28 and CTLA-4 (Yao et al., 2011, Immunity, 34: 729-740). An exemplary amino acid sequence of the ectodomain of huICOS-L is given in SEQ ID NO: 1, an exemplary amino acid sequence of murine ICOS-L (muICOS-L) is provided in SEQ ID NO:2.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins oflmmunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

Kabat et al. defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 170). Usually, a segment having the amino acid sequence of EPKSC (SEQ ID NO:171) is following to link the CH1 domain to the hinge region.

The term “hinge region” denotes the part of an antibody heavy chain polypeptide that joins in a wild-type antibody heavy chain the CH1 domain and the CH2 domain, e.g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

In one aspect, the hinge region has the amino acid sequence DKTHTCPXCP (SEQ ID NO: 172), wherein X is either S or P. In one aspect, the hinge region has the amino acid sequence HTCPXCP (SEQ ID NO: 173), wherein X is either S or P. In one aspect, the hinge region has the amino acid sequence CPXCP (SEQ ID NO: 174), wherein X is either S or P.

The term “Fc domain” or “Fe region” herein is used to define a C-terminal region of an antibody heavy chain that contains at least a portion of the constant region. The term includes native sequence Fc regions and variant Fc regions. In one aspect, a human IgG heavy chain Fc-domain extends from Cys226, or from Pro230, or from Ala231 to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, numbering according to EU index). In one aspect, a heavy chain including an Fc region as specified herein, comprised in an antibody according to the invention, comprises an additional C-terminal glycine residue (G446, numbering according to EU index). An IgG Fc region comprises an IgG CH2 and an IgG CH3 domain.

The “CH2 domain” of a human IgG Fc region usually extends from an amino acid residue at about EU position 231 to an amino acid residue at about EU position 340 (EU numbering system according to Kabat). In one aspect, a CH2 domain has the amino acid sequence of APELLGGPSV FLFPPKPKDT LMISRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 175). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one aspect, a carbohydrate chain is attached to the CH2 domain. The CH2 domain herein may be a native sequence CH2 domain or variant CH2 domain.

The “CH3 domain” comprises the stretch of residues C-terminal to a CH2 domain in an Fc region denotes the part of an antibody heavy chain polypeptide that extends approximately from EU position 341 to EU position 446 (EU numbering system according to Kabat). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO: 176). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcuRI (CD89). A particular activating Fc receptor is human FcγRIIIa (see UniProt accession no. P08637, version 141).

An “ectodomain” is the domain of a membrane protein that extends into the extracellular space (i.e. the space outside the target cell). Ectodomains are usually the parts of proteins that initiate contact with surfaces, which leads to signal transduction.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 92) GGGGSGGGGS (SEQ ID NO:93), SGGGGSGGGG (SEQ ID NO:94) and GGGGSGGGGSGGGG (SEQ ID NO:95), but also include the sequences GSPGSSSSGS (SEQ ID NO:96), (G4S)₃ (SEQ ID NO:97), (G4S)₄ (SEQ ID NO:98), GSGSGSGS (SEQ ID NO:99), GSGSGNGS (SEQ ID NO:100), GGSGSGSG (SEQ ID NO:101), GGSGSG (SEQ ID NO:102), GGSG (SEQ ID NO:103), GGSGNGSG (SEQ ID NO:104), GGNGSGSG (SEQ ID NO:105) and GGNGSG (SEQ ID NO: 106). Peptide linkers of particular interest are (G4S) (SEQ ID NO:92), (G₄S)₂ or GGGGSGGGGS (SEQ ID NO:93), (G4S)₃ (SEQ ID NO:97) and (G4S)₄ (SEQ ID NO:98).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the agonistic ICOS-binding molecules provided herein are contemplated. For example, it may be desirable to improve the binding affinity and/or other biological properties of the agonistic ICOS-binding molecules. Amino acid sequence variants of the agonistic ICOS-binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of insertions include agonistic ICOS-binding molecules with a fusion to the N- or C-terminus to a polypeptide which increases the serum half-life of the agonistic ICOS-binding molecules.

In certain embodiments, the agonistic ICOS-binding molecules provided herein are altered to increase or decrease the extent to which the antibody is glycosylated. Glycosylation variants of the molecules may be conveniently obtained by altering the amino acid sequence such that one or more glycosylation sites is created or removed. Where the agonistic ICOS-binding molecule comprises an Fc domain, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in agonistic ICOS-binding molecules may be made in order to create variants with certain improved properties. In one aspect, variants of agonistic ICOS-binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the agonistic ICOS-binding molecules of the invention include those with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the agonistic ICOS-binding molecules of the invention, e.g., the THIOMABTI antibody technology platform, in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the agonistic ICOS-binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the agonistic ICOS-binding molecules provided herein may be obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human or human patient.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. Preferably, treatment means healing of the disease or complete response. In some aspects, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

Agonistic ICOS-Binding Molecules of the Invention

The invention provides novel bispecific antigen binding molecules with particularly advantageous properties such as producibility, stability, binding affinity, biological activity, targeting efficiency, reduced toxicity, an extended dosage range that can be given to a patient and thereby a possibly enhanced efficacy.

Exemplary Agonistic ICOS-Binding Molecules Comprising at Least One Antigen Binding Domain that Binds to a Tumor-Associated Antigen

In one aspect, the invention provides bispecific agonistic ICOS-binding molecules, comprising
(a) at least one antigen binding domain capable of specific binding to ICOS, and
(b) at least one antigen binding domain capable of specific binding to a tumor-associated antigen, and
(c) a Fc domain.

In a particular aspect, the agonistic ICOS-binding molecules comprise a Fc domain comprising mutations that reduce or abolish effector function. The use of a Fc domain comprising mutations that reduce or abolish effector function will prevent unspecific agonism by crosslinking via Fc receptors and will prevent ADCC of ICOS⁺ cells.

The agonistic ICOS-binding molecules as described herein possess the advantage over conventional antibodies capable of specific binding to ICOS in that they selectively induce immune response at the target cells, which are typically cancer cells or tumor stroma. In one aspect, the tumor-associated antigen is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is FAP or CEA. In one particular aspect, the tumor-associated antigen is FAP.

In particular, the agonistic ICOS-binding molecule comprises at least one antigen binding domain capable of specific binding to FAP comprising

(a) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}FAP) comprising (i) CDR-H1 comprising the amino acid sequence of SEQ ID NO: 12, (ii) CDR-H2 comprising the amino acid sequence of SEQ ID NO: 13, and (iii) CDR-H3 comprising the amino acid sequence of SEQ ID NO: 14, and a light chain variable region (V_{L}FAP) comprising (iv) CDR-L1 comprising the amino acid sequence of SEQ ID NO: 15, (v) CDR-L2 comprising the amino acid sequence of SEQ ID NO: 16, and (vi) CDR-L3 comprising the amino acid sequence of SEQ ID NO: 17.

More particularly, the agonistic ICOS-binding molecule comprises at least one antigen binding domain capable of specific binding to FAP comprising a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9.

In a specific aspect, the agonistic ICOS-binding molecule comprises at least one antigen binding domain capable of specific binding to FAP comprising a heavy chain variable region (V_{H}FAP) comprising an amino acid sequence that is at least about 95%, 96%, 97%, 98%, 99% or 100% identical to the amino acid sequence of SEQ ID NO: 10 and a light chain variable region (V_{L}FAP) comprising an amino acid sequence that is at least about 95%, 96%, 97%, 98%, 99% or 100% identical to the amino acid sequence of SEQ ID NO: 11, or at least one antigen binding domain capable of specific binding to FAP comprising a heavy chain variable region (V_{H}FAP) comprising an amino acid sequence that is at least about 95%, 96%, 97%, 98%, 99% or 100% identical to the amino acid sequence of SEQ ID NO: 18 and a light chain variable region (V_{L}FAP) comprising an amino acid sequence that is at least about 95%, 96%, 97%, 98%, 99% or 100% identical to the amino acid sequence of SEQ ID NO: 19. In a more specific aspect, the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11 or at least one antigen binding domain capable of specific binding to FAP comprising a heavy chain variable region (V_{H}FAP) comprising an amino acid sequence of SEQ ID NO: 18 and a light chain variable region (V_{L}FAP) comprising an amino acid sequence of SEQ ID NO: 19. More particularly, the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11.

In another particular aspect, the tumor-associated antigen is CEA. In particular, the agonistic ICOS-binding molecule comprises at least one antigen binding domain capable of specific binding to CEA comprising

((a) a heavy chain variable region (V_{H}CEA) comprising (i) CDR-H1 comprising the amino acid sequence of SEQ ID NO: 145, (ii) CDR-H2 comprising the amino acid sequence of SEQ ID NO: 146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO: 148, (v) CDR-L2 comprising the amino acid sequence of SEQ ID NO: 149, and (vi) CDR-L3 comprising the amino acid sequence of SEQ ID NO: 150, or
(b) a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163.

More particularly, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO: 145, (ii) CDR-H2 comprising the amino acid sequence of SEQ ID NO: 146, and (iii) CDR-H3 comprising the amino acid sequence of SEQ ID NO: 147, and a light chain variable region (V_{L}CEA) comprising (iv) CDR-L1 comprising the amino acid sequence of SEQ ID NO: 148, (v) CDR-L2 comprising the amino acid sequence of SEQ ID NO: 149, and (vi) CDR-L3 comprising the amino acid sequence of SEQ ID NO: 150. In another aspect, the agonistic ICOS-binding molecule comprises at least one antigen binding domain capable of specific binding to CEA comprising a heavy chain variable region (V_{H}CEA) comprising (i) CDR-H1 comprising the amino acid sequence of SEQ ID NO: 158, (ii) CDR-H2 comprising the amino acid sequence of SEQ ID NO: 159, and (iii) CDR-H3 comprising the amino acid sequence of SEQ ID NO: 160, and a light chain variable region (V_{L}CEA) comprising (iv) CDR-L1 comprising the amino acid sequence of SEQ ID NO: 161, (v) CDR-L2 comprising the amino acid sequence of SEQ ID NO: 162, and (vi) CDR-L3 comprising the amino acid sequence of SEQ ID NO: 163.

In a specific aspect, the agonistic ICOS-binding molecule comprises at least one antigen binding domain capable of specific binding to CEA comprising a heavy chain variable region (V_{H}CEA) comprising an amino acid sequence that is at least about 95%, 96%, 97%, 98%, 99% or 100% identical to the amino acid sequence of SEQ ID NO: 151 and a light chain variable region (V_{L}CEA) comprising an amino acid sequence that is at least about 95%, 96%, 97%, 98%, 99% or 100% identical to the amino acid sequence of SEQ ID NO: 152, or at least one antigen binding domain capable of specific binding to FAP comprising a heavy chain variable region (V_{H}CEA) comprising an amino acid sequence that is at least about 95%, 96%, 97%, 98%, 99% or 100% identical to the amino acid sequence of SEQ ID NO: 164 and a light chain variable region (V_{L}CEA) comprising an amino acid sequence that is at least about 95%, 96%, 97%, 98%, 99% or 100% identical to the amino acid sequence of SEQ ID NO: 165. In a more specific aspect, the agonistic ICOS-binding molecule comprises at least one antigen binding domain capable of specific binding to CEA comprising a heavy chain variable region (V_{H}CEA) comprising an amino acid sequence of SEQ ID NO: 151 and a light chain variable region (V_{L}CEA) comprising an amino acid sequence of SEQ ID NO: 152 or at least one antigen binding domain capable of specific binding to CEA comprising a heavy chain variable region (V_{H}CEA) comprising an amino acid sequence of SEQ ID NO: 164 and a light chain variable region (V_{L}CEA) comprising an amino acid sequence of SEQ ID NO:165. More particularly, the agonistic ICOS-binding molecule comprises at least one antigen binding domain capable of specific binding to CEA comprising a heavy chain variable region (V_{H}CEA) comprising an amino acid sequence of SEQ ID NO: 151 and a light chain variable region (V_{L}FAP) comprising an amino acid sequence of SEQ ID NO: 152.

In a further aspect, the agonistic ICOS-binding molecule comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25.

More particularly, the agonistic ICOS-binding molecule comprising at least one antigen binding domain that binds to a tumor-associated antigen comprises at least one antigen binding domain comprising a heavy chain variable region (V_{H}ICOS) comprising an amino acid sequence that is at least about 95%, 96%, 97%, 98%, 99% or 100% identical to the amino acid sequence of SEQ ID NO:26 and a light chain variable region (V_{L}ICOS) comprising an amino acid sequence that is at least about 95%, 96%, 97%, 98%, 99% or 100% identical to the amino acid sequence of SEQ ID NO:27. More specifically, the agonistic ICOS-binding molecule comprises at least one antigen binding domain that binds to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27.

In one aspect, provided is an agonistic ICOS-binding molecule, comprising

(a) at least one antigen binding domain capable of specific binding to ICOS comprising a heavy chain variable region (V_{H}ICOS) comprising an amino acid sequence of SEQ ID NO:26 and a light chain variable region (V_{L}ICOS) comprising an amino acid sequence of SEQ ID NO:27, and
(b) at least one antigen binding domain capable of specific binding to a tumor-associated antigen comprising a heavy chain variable region (V_{H}FAP) comprising an amino acid sequence of SEQ ID NO: 10 and a light chain variable region (V_{L}FAP) comprising an amino acid sequence of SEQ ID NO: 11 or comprising a heavy chain variable region (V_{H}FAP) comprising an amino acid sequence of SEQ ID NO: 18 and a light chain variable region (V_{L}FAP) comprising an amino acid sequence of SEQ ID NO:19.

In another aspect, provided is an agonistic ICOS-binding molecule, comprising

(a) at least one antigen binding domain capable of specific binding to ICOS comprising a heavy chain variable region (V_{H}ICOS) comprising an amino acid sequence of SEQ ID NO:26 and a light chain variable region (V_{L}ICOS) comprising an amino acid sequence of SEQ ID NO:27, and
(b) at least one antigen binding domain capable of specific binding to a tumor-associated antigen comprising a heavy chain variable region (V_{H}CEA) comprising an amino acid sequence of SEQ ID NO: 151 and a light chain variable region (V_{L}CEA) comprising an amino acid sequence of SEQ ID NO: 152, or comprising a heavy chain variable region (V_{H}CEA) comprising an amino acid sequence of SEQ ID NO: 164 and a light chain variable region (V_{L}CEA) comprising an amino acid sequence of SEQ ID NO: 165.

In one aspect, the invention provides bispecific agonistic ICOS-binding molecules, comprising (a) one antigen binding domain capable of specific binding to ICOS, and (b) one antigen binding domain capable of specific binding to a tumor-associated antigen, and (c) a Fc domain. Thus, in this case the agonistic ICOS-binding molecule is monovalent for the binding to ICOS and monovalent for the binding to the tumor-associated antigen (1+1 format).

In a particular aspect, provided is an agonistic ICOS-binding molecule, wherein said molecule comprises (a) a first Fab fragment capable of specific binding to ICOS, (b) a second Fab fragment capable of specific binding to a tumor-associated antigen, and (c) a Fc domain composed of a first and a second subunit capable of stable association with each other.

More particularly, provided is a bispecific antigen binding molecule, wherein said molecule comprises

(i) a first Fab fragment capable of specific binding to ICOS, comprising a heavy chain variable region (V_{H}ICOS) comprising an amino acid sequence of SEQ ID NO:26 and a light chain variable region (V_{L}ICOS) comprising an amino acid sequence of SEQ ID NO:27, and
(ii) a second Fab fragment capable of specific binding to FAP, comprising a heavy chain variable region (V_{H}FAP) comprising an amino acid sequence of SEQ ID NO: 10 and a light chain variable region (V_{L}FAP) comprising an amino acid sequence of SEQ ID NO: 11 or comprising a heavy chain variable region (V_{H}FAP) comprising an amino acid sequence of SEQ ID NO: 18 and a light chain variable region (V_{L}FAP) comprising an amino acid sequence of SEQ ID NO: 19.

In another particular aspect, provided is a bispecific antigen binding molecule, wherein said molecule comprises

(i) a first Fab fragment capable of specific binding to ICOS, comprising a heavy chain variable region (V_{H}ICOS) comprising an amino acid sequence of SEQ ID NO:26 and a light chain variable region (V_{L}ICOS) comprising an amino acid sequence of SEQ ID NO:27, and
(ii) a second Fab fragment capable of specific binding to FAP, comprising a heavy chain variable region (V_{H}CEA) comprising an amino acid sequence of SEQ ID NO: 151 and a light chain variable region (V_{L}CEA) comprising an amino acid sequence of SEQ ID NO: 152 or comprising a heavy chain variable region (V_{H}CEA) comprising an amino acid sequence of SEQ ID NO: 164 and a light chain variable region (V_{L}CEA) comprising an amino acid sequence of SEQ ID NO:165.

In a particular aspect, provided is a bispecific antigen binding molecule comprising a first heavy chain (HC1) comprising the amino acid sequence of SEQ ID NO:28, a second heavy chain (HC2) comprising the amino acid sequence of SEQ ID NO:30, a first light chain comprising the amino acid sequence of SEQ ID NO:29 and a second light chain comprising the amino acid sequence of SEQ ID NO:31.

In one further particular aspect, provided is a bispecific antigen binding molecule comprising a first heavy chain (HC1) comprising the amino acid sequence of SEQ ID NO: 155, a second heavy chain (HC2) comprising the amino acid sequence of SEQ ID NO: 156, a first light chain comprising the amino acid sequence of SEQ ID NO:29 and a second light chain comprising the amino acid sequence of SEQ ID NO: 157.

In one aspect, provided is an agonistic ICOS-binding molecule, wherein said molecule comprises (a) a first Fab fragment capable of specific binding to ICOS, (b) a second antigen binding domain capable of specific binding to a tumor-associated antigen comprising a VH and VL domain, and (c) a Fc domain composed of a first and a second subunit capable of stable association with each other, and wherein one of the VH and VL domain of the antigen binding domain capable of specific binding to a tumor-associated antigen is fused to the C-terminus of the first subunit of the Fc domain and the other one of VH and VL is fused to the C-terminus of the second subunit of the Fc domain. Such a molecule is termed 1+1 head-to-tail.

In a particular aspect, provided is a bispecific agonistic ICOS-binding molecule comprising a first heavy chain (HC1) comprising the amino acid sequence of SEQ ID NO:32, a second heavy chain (HC2) comprising the amino acid sequence of SEQ ID NO:66, and a light chain comprising the amino acid sequence of SEQ ID NO:29.

In another aspect, the invention provides bispecific agonistic ICOS-binding molecules, comprising (a) two antigen binding domains capable of specific binding to ICOS, and (b) one antigen binding domain capable of specific binding to a tumor-associated antigen, and (c) a Fc domain. Thus, in this case the agonistic ICOS-binding molecule is bivalent for the binding to ICOS and monovalent for the binding to the tumor-associated antigen (2+1 format).

In one aspect, provided is an agonistic ICOS-binding molecule, wherein said molecule comprises (a) two Fab fragments capable of specific binding to ICOS, (b) a second antigen binding domain capable of specific binding to a tumor-associated antigen comprising a VH and VL domain, and (c) a Fc domain composed of a first and a second subunit capable of stable association with each other, and wherein one of the VH and VL domain of the antigen binding domain capable of specific binding to a tumor-associated antigen is fused to the C-terminus of the first subunit of the Fc domain and the other one of VH and VL is fused to the C-terminus of the second subunit of the Fc domain. Such a molecule is termed 2+1.

In a particular aspect, provided is a bispecific agonistic ICOS-binding molecule comprising a first heavy chain (HC1) comprising the amino acid sequence of SEQ ID NO:32, a second heavy chain (HC2) comprising the amino acid sequence of SEQ ID NO:33, and two light chains comprising the amino acid sequence of SEQ ID NO:29.

In another particular aspect, provided is a bispecific agonistic ICOS-binding molecule comprising a first heavy chain (HC1) comprising the amino acid sequence of SEQ ID NO: 153, a second heavy chain (HC2) comprising the amino acid sequence of SEQ ID NO: 154, and two light chains comprising the amino acid sequence of SEQ ID NO:29.

The invention also provides agonistic ICOS-binding molecules comprising (a) at least one ectodomain of the murine ICOS ligand, (b) one antigen binding domain capable of specific binding to the target cell antigen, and (c) a Fc domain composed of a first and a second subunit capable of stable association. In particular, the agonistic ICOS-binding molecules comprise two ectodomains of the murine ICOS ligand.

More particularly, provided is a bispecific agonistic murine ICOS-binding molecule comprising a first heavy chain (HC1) comprising the amino acid sequence of SEQ ID NO:71 and a second heavy chain (HC2) comprising the amino acid sequence of SEQ ID NO:72.

In a further aspect, a bispecific agonistic murine ICOS-binding molecule is provided, wherein the agonistic ICOS-binding molecule comprises at least one antigen binding domain capable of specific binding to murine ICOS comprising a heavy chain variable region (V_{H}ICOS) comprising (i) CDR-H1 comprising the amino acid sequence of SEQ ID NO: 123, (ii) CDR-H2 comprising the amino acid sequence of SEQ ID NO: 124, and (iii) CDR-H3 comprising the amino acid sequence of SEQ ID NO: 125, and a light chain variable region (V_{L}ICOS) comprising (iv) CDR-L1 comprising the amino acid sequence of SEQ ID NO: 126, (v) CDR-L2 comprising the amino acid sequence of SEQ ID NO: 127, and (vi) CDR-L3 comprising the amino acid sequence of SEQ ID NO: 128.

More particularly, the agonistic ICOS-binding molecule comprising at least one antigen binding domain that binds to a tumor-associated antigen comprises at least one antigen binding domain comprising a heavy chain variable region (V_{H}ICOS) comprising an amino acid sequence that is at least about 95%, 96%, 97%, 98%, 99% or 100% identical to the amino acid sequence of SEQ ID NO: 129 and a light chain variable region (V_{L}ICOS) comprising an amino acid sequence that is at least about 95%, 96%, 97%, 98%, 99% or 100% identical to the amino acid sequence of SEQ ID NO: 130. More specifically, the agonistic ICOS-binding molecule comprising at least one antigen binding domain that binds to a tumor-associated antigen comprises at least one antigen binding domain comprising a heavy chain variable region (V_{H}ICOS) comprising an amino acid sequence of SEQ ID NO: 129 and a light chain variable region (V_{L}ICOS) comprising an amino acid sequence of SEQ ID NO:130.

In a particular aspect, provided is a bispecific agonistic ICOS-binding molecule comprising a first heavy chain (HC1) comprising the amino acid sequence of SEQ ID NO: 166, a second heavy chain (HC2) comprising the amino acid sequence of SEQ ID NO: 167, and two light chains comprising the amino acid sequence of SEQ ID NO: 131.

In another particular aspect, provided is a bispecific agonistic ICOS-binding molecule comprising a first heavy chain (HC1) comprising the amino acid sequence of SEQ ID NO: 137, a second heavy chain (HC2) comprising the amino acid sequence of SEQ ID NO: 168, a first light chain comprising the amino acid sequence of SEQ ID NO: 131 and a second light chain comprising the amino acid sequence of SEQ ID NO: 169.

Fc Domain Modifications Reducing Fc Receptor Binding and/or Effector Function

The Fc domain of the agonistic ICOS-binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

Thus, the agonistic ICOS-binding molecule comprising at least one antigen binding domain that binds to a tumor-associated antigen comprises an IgG Fc domain, specifically an IgG1 Fc domain or an IgG4 Fc domain. More particularly, the agonistic ICOS-binding molecule comprising at least one antigen binding domain that binds to a tumor-associated antigen comprises an IgG1 Fc domain.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the agonistic ICOS-binding molecules of the invention exhibits reduced binding affinity to an Fc receptor and/or reduced effector function, as compared to a native IgG1 Fc domain. In one aspect, the Fc domain does not substantially bind to an Fc receptor and/or does not induce effector function. In a particular aspect, the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc domain of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides an antibody, wherein the Fc domain comprises one or more amino acid substitution that reduces binding to an Fc receptor, in particular towards Fcγ receptor.

In one aspect, the Fc domain of the antibody of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, Kabat EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

The following section describes preferred aspects of the agonistic ICOS-binding molecules of the invention comprising Fc domain modifications that reduce Fc receptor binding and/or effector function. In one aspect, the invention relates to the bispecific antigen binding molecule (a) at least one antigen binding domain capable of specific binding to ICOS, (b) at least one antigen binding domain capable of specific binding to a tumor-associated antigen, and (c) a Fc domain composed of a first and a second subunit capable of stable association, wherein the Fc domain comprises one or more amino acid substitution that reduces the binding affinity of the antibody to an Fc receptor, in particular towards Fcγ receptor. In another aspect, the invention relates to the agonistic ICOS-binding molecule comprising (a) at least one antigen binding domain capable of specific binding to ICOS, (b) at least one antigen binding domain capable of specific binding to a target cell antigen, and (c) a Fc domain composed of a first and a second subunit capable of stable association, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

In one aspect of the invention, the Fc region comprises an amino acid substitution at positions D265, and P329. In some aspects, the Fc region comprises the amino acid substitutions D265A and P329G (“DAPG”) in the CH2 domain. In one such embodiment, the Fc region is an IgG1 Fc region, particularly a mouse IgG1 Fc region. DAPG mutations are described e.g. in WO 2016/030350 A1, and can be introduced in CH2 regions of heavy chains to abrogate binding of antigen binding molecules to murine Fc gamma receptors.

Fc Domain Modifications Promoting Heterodimerization

The agonistic ICOS-binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the agonistic ICOS-binding molecules of the invention in recombinant production, it will thus be advantageous to introduce in the Fc domain of the bispecific antigen binding molecules of the invention a modification promoting the association of the desired polypeptides.

Accordingly, in particular aspects the invention relates to agonistic ICOS-binding molecules comprising (a) at least one antigen binding domain capable of specific binding to ICOS, (b) at least one antigen binding domain capable of specific binding to a tumor-associated antigen, and (c) a Fc domain composed of a first and a second subunit capable of stable association with each other, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, the invention relates to the agonistic ICOS-binding molecule comprising (a) at least one antigen binding domain capable of specific binding to ICOS, (b) at least one antigen binding domain capable of specific binding to a tumor-associated antigen, and (c) a Fc domain composed of a first and a second subunit capable of stable association with each other, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the agonistic ICOS-binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in the formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In one aspect, the first subunit of the Fc region comprises aspartic acid residues (D) at positions 392 and 409, and the second subunit of the Fc region comprises lysine residues (K) at positions 356 and 399. In some embodiments, in the first subunit of the Fc region the lysine residues at positions 392 and 409 are replaced with aspartic acid residues (K392D, K409D), and in the second subunit of the Fc region the glutamate residue at position 356 and the aspartic acid residue at position 399 are replaced with lysine residues (E356K, D399K). “DDKK” knob-into-hole technology is described e.g. in WO 2014/131694 A1, and favours the assembly of the heavy chains bearing subunits providing the complementary amino acid residues.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

Exemplary Anti-CEA/Anti-CD3 Bispecific Antibodies for Use in the Invention

The present invention relates to anti-CEA/anti-CD3 bispecific antibodies and their use in combination with agonistic ICOS-binding molecules, in particular to their use in a method for treating or delaying progression of cancer, more particularly for treating or delaying progression of solid tumors. The anti-CEA/anti-CD3 bispecific antibodies as used herein are bispecific antibodies comprising a first antigen binding domain that binds to CD3, and a second antigen binding domain that binds to CEA.

Thus, the anti-CEA/anti-CD3 bispecific antibody as used herein comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

In a particular aspect, the anti-CEA/anti-CD3 bispecific antibody for use in the combination comprises a first antigen binding domain comprising a heavy chain variable region (V_{H}CD3) comprising CDR-H1 sequence of SEQ ID NO:33, CDR-H2 sequence of SEQ ID NO:34, and CDR-H3 sequence of SEQ ID NO:35; and/or a light chain variable region (V_{L}CD3) comprising CDR-L1 sequence of SEQ ID NO:36, CDR-L2 sequence of SEQ ID NO:37, and CDR-L3 sequence of SEQ ID NO:38. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:39 and/or a light chain variable region (V_{L}CD3) that is at least 90%, 95%, 96%, 97%, 98%, or 99% identical to the amino acid sequence of SEQ ID NO:40. In a further aspect, the anti-CEA/anti-CD3 bispecific antibody comprises a heavy chain variable region (V_{H}CD3) comprising the amino acid sequence of SEQ ID NO:39 and/or a light chain variable region (V_{L}CD3) comprising the amino acid sequence of SEQ ID NO:40.

In one aspect, the antibody that capable of specifically binding to CD3 is a full-length antibody. In one aspect, the antibody that capable of specifically binding to CD3 is an antibody of the human IgG class, particularly an antibody of the human IgG1 class. In one aspect, the antibody that capable of specifically binding to CD3 is an antibody fragment, particularly a Fab molecule or a scFv molecule, more particularly a Fab molecule. In a particular aspect, the antibody that capable of specifically binding to CD3 is a crossover Fab molecule wherein the variable domains or the constant domains of the Fab heavy and light chain are exchanged (i.e. replaced by each other). In one aspect, the antibody that capable of specifically binding to CD3 is a humanized antibody.

In another aspect, the anti-CEA/anti-CD3 bispecific antibody comprises a second antigen binding domain comprising

(a) a heavy chain variable region (V_{H}CEA) comprising CDR-H1 sequence of SEQ ID NO:41, CDR-H2 sequence of SEQ ID NO:42, and CDR-H3 sequence of SEQ ID NO:43, and/or a light chain variable region (V_{L}CEA) comprising CDR-L1 sequence of SEQ ID NO:44, CDR-L2 sequence of SEQ ID NO:45, and CDR-L3 sequence of SEQ ID NO:46, or
(b) a heavy chain variable region (V_{H}CEA) comprising CDR-H1 sequence of SEQ ID NO:49, CDR-H2 sequence of SEQ ID NO:50, and CDR-H3 sequence of SEQ ID NO:51, and/or a light chain variable region (V_{L}CEA) comprising CDR-L1 sequence of SEQ ID NO:52, CDR-L2 sequence of SEQ ID NO:53, and CDR-L3 sequence of SEQ ID NO:54.

More particularly, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:47 and/or a light chain variable region (V_{L}CEA) that is at least 90%, 95%, 96%, 97%, 98%, or 99% identical to the amino acid sequence of SEQ ID NO:48. In a further aspect, the anti-CEA/anti-CD3 bispecific comprises a second antigen binding domain comprising a heavy chain variable region (V_{H}CEA) comprising the amino acid sequence of SEQ ID NO:47 and/or a light chain variable region (V_{L}CEA) comprising the amino acid sequence of SEQ ID NO:48. In another aspect, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:55 and/or a light chain variable region (V_{L}CEA) that is at least 90%, 95%, 96%, 97%, 98%, or 99% identical to the amino acid sequence of SEQ ID NO:56. In a further aspect, the anti-CEA/anti-CD3 bispecific comprises a second antigen binding domain comprising a heavy chain variable region (V_{H}CEA) comprising the amino acid sequence of SEQ ID NO:55 and/or a light chain variable region (V_{L}CEA) comprising the amino acid sequence of SEQ ID NO:56.

In another particular aspect, the anti-CEA/anti-CD3 bispecific antibody comprises a third antigen binding domain that binds to CEA. In particular, the anti-CEA/anti-CD3 bispecific antibody comprises a third antigen binding domain comprising

(a) a heavy chain variable region (V_{H}CEA) comprising CDR-H1 sequence of SEQ ID NO:41, CDR-H2 sequence of SEQ ID NO:42, and CDR-H3 sequence of SEQ ID NO:43, and/or a light chain variable region (V_{L}CEA) comprising CDR-L1 sequence of SEQ ID NO:44, CDR-L2 sequence of SEQ ID NO:45, and CDR-L3 sequence of SEQ ID NO:46, or
(b) a heavy chain variable region (V_{H}CEA) comprising CDR-H1 sequence of SEQ ID NO:49, CDR-H2 sequence of SEQ ID NO:50, and CDR-H3 sequence of SEQ ID NO:51, and/or a light chain variable region (V_{L}CEA) comprising CDR-L1 sequence of SEQ ID NO:52, CDR-L2 sequence of SEQ ID NO:53, and CDR-L3 sequence of SEQ ID NO:54.

More particularly, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:47 and/or a light chain variable region (V_{L}CEA) that is at least 90%, 95%, 96%, 97%, 98%, or 99% identical to the amino acid sequence of SEQ ID NO:48. In a further aspect, the anti-CEA/anti-CD3 bispecific comprises a third antigen binding domain comprising a heavy chain variable region (V_{H}CEA) comprising the amino acid sequence of SEQ ID NO:47 and/or a light chain variable region (V_{L}CEA) comprising the amino acid sequence of SEQ ID NO:48. In another particular aspect, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:55 and/or a light chain variable region (V_{L}CEA) that is at least 90%, 95%, 96%, 97%, 98%, or 99% identical to the amino acid sequence of SEQ ID NO:56. In a further aspect, the anti-CEA/anti-CD3 bispecific comprises a third antigen binding domain comprising a heavy chain variable region (V_{H}CEA) comprising the amino acid sequence of SEQ ID NO:55 and/or a light chain variable region (V_{L}CEA) comprising the amino acid sequence of SEQ ID NO:56.

In a further aspect, the anti-CEA/anti-CD3 bispecific antibody is a bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In another aspect, the anti-CEA/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)n, (SG₄)n, (G₄S)ₙ or G₄(SG₄)ₙ peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)nGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2. In one embodiment said peptide linker is (G₄S)₂. A particularly suitable peptide linker for fusing the Fab light chains of the first and the second Fab molecule to each other is (G₄S)₂. An exemplary peptide linker suitable for connecting the Fab heavy chains of the first and the second Fab fragments comprises the sequence (D)-(G₄S)₂. Another suitable such linker comprises the sequence (G₄S)₄. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-CEA/anti-CD3 bispecific antibody comprises an Fc domain comprising one or more amino acid substitutions that reduce binding to an Fc receptor and/or effector function. In particular, the anti-CEA/anti-CD3 bispecific antibody comprises an IgG1 Fc domain comprising the amino acid substitutions L234A, L235A and P329G.

In a particular aspect, the anti-CEA/anti-CD3 bispecific antibody comprises a polypeptide that is at least 95%, 96%, 97%, 98%, or 99% identical to the sequence of SEQ ID NO: 61, a polypeptide that is at least 95%, 96%, 97%, 98%, or 99% identical to the sequence of SEQ ID NO: 62, a polypeptide that is at least 95%, 96%, 97%, 98%, or 99% identical to the sequence of SEQ ID NO: 63, and a polypeptide that is at least 95%, 96%, 97%, 98%, or 99% identical to the sequence of SEQ ID NO: 64. In a further particular embodiment, the bispecific antibody comprises a polypeptide sequence of SEQ ID NO: 61, a polypeptide sequence of SEQ ID NO: 62, a polypeptide sequence of SEQ ID NO: 63 and a polypeptide sequence of SEQ ID NO: 64 (CEA CD3 TCB).

In a further particular aspect, the anti-CEA/anti-CD3 bispecific antibody comprises a polypeptide that is at least 95%, 96%, 97%, 98%, or 99% identical to the sequence of SEQ ID NO:57, a polypeptide that is at least 95%, 96%, 97%, 98%, or 99% identical to the sequence of SEQ ID NO:58, a polypeptide that is at least 95%, 96%, 97%, 98%, or 99% identical to the sequence of SEQ ID NO:59, and a polypeptide that is at least 95%, 96%, 97%, 98%, or 99% identical to the sequence of SEQ ID NO:60. In a further particular embodiment, the bispecific antibody comprises a polypeptide sequence of SEQ ID NO:57, a polypeptide sequence of SEQ ID NO:58, a polypeptide sequence of SEQ ID NO:59 and a polypeptide sequence of SEQ ID NO:60 (CEACAM5 CD3 TCB).

Particular bispecific antibodies are described in PCT publication no. WO 2014/131712 A1.

In a further aspect, the anti-CEA/anti-CD3 bispecific antibody may also comprise a bispecific T cell engager (BiTE®@). In a further aspect, the anti-CEA/anti-CD3 bispecific antibody is a bispecific antibody as described in WO 2007/071426 or WO 2014/131712. In another aspect, the bispecific antibody is MEDI565.

In another aspect, the invention relates to a murine anti-CEA/anti-CD3 bispecific antibody comprising a first antigen binding domain comprising a heavy chain variable region (V_{H}muCD3) and a light chain variable region (V_{L}muCD3), a second antigen binding domain comprising a heavy chain variable region (V_{H}muCEA) and a light chain variable region (V_{L}muCEA) and a third antigen binding domain comprising a heavy chain variable region (V_{H}muCEA) and a light chain variable region (V_{L}muCEA).

In a particular aspect, the murine anti-CEA/anti-CD3 bispecific antibody comprises a polypeptide that is at least 95%, 96%, 97%, 98%, or 99% identical to the sequence of SEQ ID NO:75, a polypeptide that is at least 95%, 96%, 97%, 98%, or 99% identical to the sequence of SEQ ID NO: 62, a polypeptide that is at least 95%, 96%, 97%, 98%, or 99% identical to the sequence of SEQ ID NO:76, a polypeptide that is at least 95%, 96%, 97%, 98%, or 99% identical to the sequence of SEQ ID NO:77 and a polypeptide that is at least 95%, 96%, 97%, 98%, or 99% identical to the sequence of SEQ ID NO:78. In a further particular aspect, the murine anti-CEA/anti-CD3 bispecific antibody comprises a polypeptide sequence of SEQ ID NO:75, a polypeptide sequence of SEQ ID NO:76, a polypeptide sequence of SEQ ID NO:77 and a polypeptide sequence of SEQ ID NO:78 (mu CEA CD3 TCB).

Agents Blocking PD-L1/PD-1 Interaction for Use in the Invention

In one aspect of the invention, the T-cell activating anti-CD3 bispecific antibodies specific for a tumor-associated antigen, in particular the anti-CEA/anti-CD3 antibodies are for use in a method for treating or delaying progression of cancer, wherein the T-cell activating anti-CD3 bispecific antibodies specific for a tumor-associated antigen are used in combination with a 4-1BB (CD137) agonist and additionally they are combined with an agent blocking PD-L1/PD-1 interaction. In another aspect, the agent blocking PD-L1/PD-1 interaction is only combined with a targeted 4-1BB agonist. In all these aspects, an agent blocking PD-L1/PD-1 interaction is a PD-L1 binding antagonist or a PD-1 binding antagonist. In particular, the agent blocking PD-L1/PD-1 interaction is an anti-PD-L1 antibody or an anti-PD-1 antibody.

The term “PD-L1”, also known as CD274 or B7-H1, refers to any native PD-L1 from any vertebrate source, including mammals such as primates (e.g. humans) non-human primates (e.g. cynomolgus monkeys) and rodents (e.g. mice and rats), in particular to “human PD-L1”. The amino acid sequence of complete human PD-L1 is shown in UniProt (www.uniprot.org) accession no. Q9NZQ7 (SEQ ID NO: 107). The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In particular, a PD-L1 binding antagonist is an anti-PD-L1 antibody. The term “anti-PD-L1 antibody” or “antibody binding to human PD-L1” or “antibody that capable of specifically binding to human PD-L1” or “antagonistic anti-PD-L1” refers to an antibody specifically binding to the human PD-L1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In a particular aspect, the agent blocking PD-L1/PD-1 interaction is an anti-PD-L1 antibody. In a specific aspect, the anti-PD-L1 antibody is selected from the group consisting of atezolizumab (MPDL3280A, RG7446), durvalumab (MEDI4736), avelumab (MSB0010718C) and MDX-1105. In a specific aspect, an anti-PD-L1 antibody is YW243.55.570 described herein. In another specific aspect, an anti-PD-L1 antibody is MDX-1105 described herein. In still another specific aspect, an anti-PD-L1 antibody is MEDI4736 (durvalumab). In yet a further aspect, an anti-PD-L1 antibody is MSB0010718C (avelumab). More particularly, the agent blocking PD-L1/PD-1 interaction is atezolizumab (MPDL3280A). In another aspect, the agent blocking PD-L1/PD-1 interaction is an anti-PD-L1 antibody comprising a heavy chain variable domain VH(PDL-1) of SEQ ID NO:109 and a light chain variable domain VL(PDL-1) of SEQ ID NO: 110. In another aspect, the agent blocking PD-L1/PD-1 interaction is an anti-PD-L1 antibody comprising a heavy chain variable domain VH(PDL-1) of SEQ ID NO: 111 and a light chain variable domain VL(PDL-1) of SEQ ID NO: 112.

The term “PD-1”, also known as CD279, PD1 or programmed cell death protein 1, refers to any native PD-L1 from any vertebrate source, including mammals such as primates (e.g. humans) non-human primates (e.g. cynomolgus monkeys) and rodents (e.g. mice and rats), in particular to the human protein PD-1 with the amino acid sequence as shown in UniProt (www.uniprot.org) accession no. Q15116 (SEQ ID NO: 108). The term “PD-1 binding antagonist” refers to a molecule that inhibits the binding of PD-1 to its ligand binding partners. In some embodiments, the PD-1 binding antagonist inhibits the binding of PD-1 to PD-L1. In some embodiments, the PD-1 binding antagonist inhibits the binding of PD-1 to PD-L2. In some embodiments, the PD-1 binding antagonist inhibits the binding of PD-1 to both PD-L1 and PD-L2. In particular, a PD-L1 binding antagonist is an anti-PD-L1 antibody. The term “anti-PD-1 antibody” or “antibody binding to human PD-1” or “antibody that capable of specifically binding to human PD-1” or “antagonistic anti-PD-1” refers to an antibody specifically binding to the human PD1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In one aspect, the agent blocking PD-L1/PD-1 interaction is an anti-PD-1 antibody. In a specific aspect, the anti-PD-1 antibody is selected from the group consisting of MDX 1106 (nivolumab), MK-3475 (pembrolizumab), CT-011 (pidilizumab), MEDI-0680 (AMP-514), PDR001, REGN2810, and BGB-108, in particular from pembrolizumab and nivolumab. In another aspect, the agent blocking PD-L1/PD-1 interaction is an anti-PD-1 antibody comprising a heavy chain variable domain VH(PD-1) of SEQ ID NO: 113 and a light chain variable domain VL(PD-1) of SEQ ID NO: 114. In another aspect, the agent blocking PD-L1/PD-1 interaction is an anti-PD-1 antibody comprising a heavy chain variable domain VH(PD-1) of SEQ ID NO: 115 and a light chain variable domain VL(PD-1) of SEQ ID NO: 116.

Polynucleotides

The invention further provides isolated polynucleotides encoding agonistic ICOS-binding molecule or a T-cell bispecific antibody as described herein or a fragment thereof.

The isolated polynucleotides encoding the bispecific antibodies of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire antigen-binding molecule according to the invention as described herein. In other embodiments, the isolated polynucleotide encodes a polypeptide comprised in the antibody according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Bispecific antibodies of the invention may be obtained, for example, by solid-state peptide synthesis (e.g. Merrifield solid phase synthesis) or recombinant production. For recombinant production one or more polynucleotide encoding the antibody or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody or polypeptide fragments thereof is desired, DNA encoding a signal sequence may be placed upstream of the nucleic acid an antibody of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse 0-glucuronidase.

DNA encoding a short protein sequence that could be used to facilitate later purification (e.g. a histidine tag) or assist in labeling the fusion protein may be included within or at the ends of the polynucleotide encoding a bispecific antibody of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an antibody of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr− CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NSO, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an agonistic ICOS-binding molecule of the invention or polypeptide fragments thereof is provided, wherein the method comprises culturing a host cell comprising polynucleotides encoding the agonistic ICOS-binding molecule or polypeptide fragments thereof, as provided herein, under conditions suitable for expression of the antibody of the invention or polypeptide fragments thereof, and recovering the antibody of the invention or polypeptide fragments thereof from the host cell (or host cell culture medium).

In certain embodiments the antigen binding domains capable of specific binding to a tumor-associated antigen or antigen binding domains capable of specific binding to ICOS (e.g. Fab fragments or VH and VL) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e.g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osboum et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the antikgne binding domains are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, N.Y.).

Agonistic ICOS-binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art.

1. Affinity Assays

The affinity of the antibody provided herein for ICOS or the tumor-associated antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the bispecific antigen binding molecule for the target cell antigen can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 9. According to one aspect, K_{D} is measured by surface plasmon resonance using a BIACORE® T100 machine (GE Healthcare) at 25° C.

2. Binding Assays and Other Assays

In one aspect, an antibody as reported herein is tested for its antigen binding activity, e.g., by known methods such as ELISA, Western blot, flow cytometry, etc.

3. Activity Assays

Several cell-based in vitro assays were performed to evaluate the activity of the agonistic ICOS-binding molecules comprising at least one antigen binding domain that binds to a tumor-associated antigen. The assays were designed to show additional agonistic/co-stimulatory activity of the anti-ICOS bispecific molecules in presence of T-cell bispecific-(TCB) mediated activation of T-cells. For example, a Jurkat assay with a reporter cell line with NFAT-regulated expression of luciferase, induced upon engagement of the CD3/TCR and ICOS), wherein ICOS IgG molecules, plate-bound vs. in solution and in absence versus presence of a coated CD3 IgG stimulus were measured, is described in more detail in Example 11,

Furthermore, primary human PBMC co-culture assays, wherein FAP-targeted ICOS molecules, cross-linked by simultaneous binding to human ICOS on T-cells and human FAP, expressed on 3T3-hFAP cells (parental cell line ATCC # CCL-92, modified to stably overexpress human FAP), in the presence of a TCB molecule being crosslinked by simultaneous binding to CD3 on T-cells and human CEA on tumor cells were tested and described in Example 12. A primary murine splenocyte co-culture assay is described in Example 13.

In certain aspects, an antibody as reported herein is tested for such biological activity.

Pharmaceutical Compositions, Formulations and Routes of Administration

In a further aspect, the invention provides pharmaceutical compositions comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients. In a particular aspect, there is provided a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients for use in the treatment of cancer, more particularly for the treatment of solid tumors. In one further aspect, provided a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen, wherein the agonistic ICOS-binding molecule comprising at least one antigen binding domain capable of specific binding to a tumor-associated antigen and the T-cell activating anti-CD3 bispecific antibody specific for a tumor-associated antigen are for administration together in a single composition or for separate administration in two or more different compositions. In another aspect, the agonistic ICOS-binding molecule comprising at least one antigen binding domain capable of specific binding to a tumor-associated antigen is administered concurrently with, prior to, or subsequently to the T-cell activating anti-CD3 bispecific antibody specific for a tumor-associated antigen.

In another aspect, a pharmaceutical composition comprises an agonistic ICOS-binding molecule provided herein and at least one pharmaceutically acceptable excipient. In another aspect, a pharmaceutical composition comprises an agonistic ICOS-binding molecule provided herein and at least one additional therapeutic agent, e.g., as described below.

In yet another aspect, the invention provides a pharmaceutical composition comprising an agonistic ICOS-binding molecule comprising at least one antigen binding domain capable of specific binding to a tumor-associated antigen for use in a method for treating or delaying progression of cancer, wherein the agonistic ICOS-binding molecule comprising at least one antigen binding domain capable of specific binding to a tumor-associated antigen is for use in combination with a T-cell activating anti-CD3 bispecific antibody specific for a tumor-associated antigen or for combination with an agent blocking PD-L1/PD-1 interaction. In another aspect, the agonistic ICOS-binding molecule comprising at least one antigen binding domain capable of specific binding to a tumor-associated antigen is for use in combination with a T-cell activating anti-CD3 bispecific antibody specific for a tumor-associated antigen and in ombination with an agent blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab. In another specific aspect, the agent blocking PD-L1/PD-1 interaction is pembrolizumab or nivolumab.

Pharmaceutical compositions of the present invention comprise a therapeutically effective amount of one or more antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the agonistic ICOS-binding molecules described herein may also be formulated as a depot preparation. Such long acting formulations may be administered by implantation (for example subcutaneously or intramuscularly) or by intramuscular injection. Thus, for example, the agonistic ICOS-binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the agonistic ICOS-binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The agonistic ICOS-binding molecule of the invention may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended. The formulations to be used for in vivo administration are generally sterile. Sterility may be readily accomplished, e.g., by filtration through sterile filtration membranes.

Therapeutic Methods and Compositions

In one aspect, provided is a method for treating or delaying progression of cancer in a subject comprising administering to the subject an effective amount of an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody.

In one such aspect, the method further comprises administering to the subject an effective amount of at least one additional therapeutic agent. In further embodiments, herein is provided a method for tumor shrinkage comprising administering to the subject an effective amount of an agonistic ICOS-binding molecule comprising at least one antigen binding domain capable of specific binding to a tumor-associated antigen and a T-cell activating anti-CD3 bispecific antibody, in particular an anti-CEA/anti-CD3 bispecific antibody. An “individual” or a “subject” according to any of the above aspects is preferably a human.

In further aspects, a composition for use in cancer immunotherapy is provided comprising an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody. In certain embodiments, a composition comprising an agonistic ICOS-binding molecule comprising at least one antigen binding domain capable of specific binding to a tumor-associated antigen and a T-cell activating anti-CD3 bispecific antibody, in particular an anti-CEA/anti-CD3 bispecific antibody, for use in a method of cancer immunotherapy is provided.

In a further aspect, herein is provided the use of a composition comprising an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody, in the manufacture or preparation of a medicament. In one aspect, the medicament is for treatment of cancer. In a further aspect, the medicament is for use in a method of tumor shrinkage comprising administering to an individual having a solid tumor an effective amount of the medicament. In one such aspect, the method further comprises administering to the individual an effective amount of at least one additional therapeutic agent. In a further embodiment, the medicament is for treating solid tumors. In some aspects, the individual has CEA positive cancer. In some aspects, CEA positive cancer is colon cancer, lung cancer, ovarian cancer, gastric cancer, bladder cancer, pancreatic cancer, endometrial cancer, breast cancer, kidney cancer, esophageal cancer, or prostate cancer. In some aspects, the breast cancer is a breast carcinoma or a breast adenocarcinoma. In some aspects, the breast carcinoma is an invasive ductal carcinoma. In some aspects, the lung cancer is a lung adenocarcinoma. In some embodiments, the colon cancer is a colorectal adenocarcinoma. A “subject” or an “individual” according to any of the above embodiments may be a human.

In another aspect, provided is a method for treating or delaying progression of cancer in a subject comprising administering to the subject an effective amount of an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody, wherein the subject comprises a low ICOS baseline expression on T cells before treatment with the agonistic ICOS-binding molecule.

The combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one aspect, administration of a T-cell activating anti-CD3 bispecific antibody, in particular a anti-CEA/anti-CD3 bispecific antibody, and of an agonistic ICOS-binding molecule comprising at least one antigen binding domain capable of specific binding to a tumor-associated antigen and optionally the administration of an additional therapeutic agent occur within about one month, or within about one, two or three weeks, or within about one, two, three, four, five, or six days, of each other.

Both the T-cell activating anti-CD3 bispecific antibody, in particular an anti-CEA/anti-CD3 bispecific antibody, and the agonistic ICOS-binding molecule comprising at least one antigen binding domain capable of specific binding to a tumor-associated antigen as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Both the T-cell activating anti-CD3 bispecific antibody, in particular an anti-CEA/anti-CD3 bispecific antibody, and the agonistic ICOS-binding molecule comprising at least one antigen binding domain capable of specific binding to a tumor-associated antigen as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In another aspect, provided is a method for treating or delaying progression of cancer in a subject comprising administering to the subject an effective amount of an agonistic ICOS-binding molecule comprising at least one antigen binding domain capable of specific binding to a tumor-associated antigen.

Other Agents and Treatments

The agonistic ICOS-binding molecules comprising at least one antigen binding domain capable of specific binding to a tumor-associated antigen of the invention may be administered in combination with one or more other agents in therapy. For instance, an agonistic ICOS-binding molecules of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent. In one aspect, the additional therapeutic agent is selected from the group consisting of a chemotherapeutic agent, radiation and other agents for use in cancer immunotherapy. In a further aspect, provided is the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain that binds to a tumor-associated antigen is administered in combination with another immunomodulator.

The term “immunomodulator” refers to any substance including a monoclonal antibody that effects the immune system. The molecules of the inventions can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), OX-40 antibodies, LAG3 antibodies, TIM-3 antibodies, 4-1BB antibodies and GITR antibodies.

In a further aspect, provided is the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain capable of specific binding to a tumor-associated antigen is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In one specific aspect, the agent blocking PD-L1/PD-1 interaction is atezolizumab. In another aspect, the agent blocking PD-L1/PD-1 interaction is pembrolizumab or nivolumab. Such other agents are suitably present in combination in amounts that are effective for the purpose intended. The effective amount of such other agents depends on the amount of agonistic ICOS-binding molecule used, the type of disorder or treatment, and other factors discussed above. The agonistic ICOS-binding molecules comprising at least one antigen binding domain capable of specific binding to a tumor-associated antigen are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the agonistic ICOS-binding molecules comprising at least one antigen binding domain capable of specific binding to a tumor-associated antigen of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an agonistic ICOS-binding molecule comprising at least one antigen binding domain capable of specific binding to a tumor-associated antigen of the invention.

The label or package insert indicates that the composition is used for treating the condition of choice. Moreover, the article of manufacture may comprise (a) a first container with a composition contained therein, wherein the composition comprises an agonistic ICOS-binding molecule comprising at least one antigen binding domain capable of specific binding to a tumor-associated antigen of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID | | |
| NO: | Name | Sequence |
| 1 | huICOS-L(ECD) | DTQEKEVRAM VGSDVELSCA CPEGSRFDLN DVYVYWQTSE |
| | | SKTVVTYHIP QNSSLENVDS RYRNRALMSP AGMLRGDFSL |
| | | RLFNVTPQDE QKFHCLVLSQ SLGFQEVLSV EVTLHVAANF |
| | | SVPVVSAPHS PSQDELTFTC TSINGYPRPN VYWINKTDNS |
| | | LLDQALQNDT VFLNMRGLYD VVSVLRIART PSVNIGCCIE |
| | | NVLLQQNLTV GSQTGNDIGE RDKITENPVS TGEKNAAT |
| 2 | muICOS-L | ETEVGAMVGS NVVLSCIDPH RRHFNLSGLY VYWQIENPEV |
| | | SVTYYLPYKS PGINVDSSYK NRGHLSLDSM KQGNFSLYLK |
| | | NVTPQDTQEF TCRVFMNTAT ELVKILEEVV RLRVAANFST |
| | | PVISTSDSSN PGQERTYTCM SKNGYPEPNL YWINTTDNSL |
| | | IDTALQNNTV YLNKLGLYDV ISTLRLPWTS RGDVLCCVEN |
| | | VALHQNITSI SQAESFTGNN TKNPQETHNN ELK |
| 3 | human ICOS | UniProt Q9Y6W8: |
| | | MKSGLWYFFL FCLRIKVLTG EINGSANYEM FIFHNGGVQI |
| | | LCKYPDIVQQ FKMQLLKGGQ ILCDLTKTKG SGNTVSIKSL |
| | | KFCHSQLSNN SVSFFLYNLD HSHANYYFCN LSIFDPPPFK |
| | | VTLTGGYLHI YESQLCCQLK FWLPIGCAAF VVVCILGCIL |
| | | ICWLTKKKYS SSVHDPNGEY MFMRAVNTAK KSRLTDVTL |
| 4 | FAP(4B9) CDR-H1 | SYAMS |
| 5 | FAP(4B9) CDR-H2 | AIIGSGASTYYADSVKG |
| 6 | GAP(4B9) CDR-H3 | GWFGGFNY |
| 7 | FAP(4B9) CDR-L1 | RASQSVTSSYLA |
| 8 | FAP(4B9) CDR-L2 | VGSRRAT |
| 9 | FAP(4B9) CDR-L3 | QQGIMLPPT |
| 10 | FAP(4B9) VH | EVQLLESGGGLVQPGGSLRLSCAASGFTFSSYAMSWVRQA |
| | | PGKGLEWVSAIIGSGASTYYADSVKGRFTISRDNSKNTLY |
| | | LQMNSLRAEDTAVYYCAKGWFGGFNYWGQGTLVTVSS |
| 11 | FAP(4B9) VL | EIVLTQSPGTLSLSPGERATLSCRASQSVTSSYLAWYQQK |
| | | PGQAPRLLINVGSRRATGIPDRFSGSGSGTDFTLTISRLE |
| | | PEDFAVYYCQQGIMLPPTFGQGTKVEIK |
| 12 | FAP(28H1) CDR-H1 | SHAMS |
| 13 | FAP(28H1) CDR-H2 | AIWASGEQYYADSVKG |
| 14 | FAP(28H1) CDR-H3 | GWLGNFDY |
| 15 | FAP(28H1) CDR-L1 | RASQSVSRSYLA |
| 16 | FAP(28H1) CDR-L2 | GASTRAT |
| 17 | FAP(28H1) CDR-L3 | QQGQVIPPT |
| 18 | FAP(28H1) VH | EVQLLESGGGLVQPGGSLRLSCAASGFTFSSHAMSWVRQA |
| | | PGKGLEWVSAIWASGEQYYADSVKGRFTISRDNSKNTLYL |
| | | QMNSLRAEDTAVYYCAKGWLGNFDYWGQGTLVTVSS |
| 19 | FAP(28H1) VL | EIVLTQSPGTLSLSPGERATLSCRASQSVSRSYLAWYQQK |
| | | PGQAPRLLIIGASTRATGIPDRFSGSGSGTDFTLTISRLE |
| | | PEDFAVYYCQQGQVIPPTFGQGTKVEIK |
| 20 | ICOS CDR-H1 | GYTFTGYYMH |
| 21 | ICOS CDR-H2 | WINPHSGGTNYAQKFQG |
| 22 | ICOS CDR-H3 | TYYYDSSGYYHDAFDI |
| 23 | ICOS CDR-L1 | RASQGISRLLA |
| 24 | ICOS CDR-L2 | VASSLQS |
| 25 | ICOS CDR-L3 | QQANSFPWT |
| 26 | ICOS(JMAb136) VH | QVQLVQSGAEVKKPGASVKVSCKASGYTFTGYYMHWVRQA |
| | | PGQGLEWMGWINPHSGGTNYAQKFQGRVTMTRDTSISTAY |
| | | MELSRLRSDDTAVYYCARTYYYDSSGYYHDAFDIWGQGTM |
| | | VTVSS |
| 27 | ICOS(JMAb136) VL | DIQMTQSPSSVSASVGDRVTITCRASQGISRLLAWYQQKP |
| | | GKAPKLLIYVASSLQSGVPSRFSGSGSGTDFTLTISSLQP |
| | | EDFATYYCQQANSFPWTFGQGTKVEIK |
| 28 | VHCH1(JMAb136)-Fc | see Table 1 |
| | knob chain | |
| 29 | VLCL(JMAb136) Light | see Table 1 |
| | chain | |
| 30 | VHCH1(4B9)-Fc hole | see Table 1 |
| | chain | |
| 31 | VLCL(4B9) Light chain | see Table 1 |
| 32 | VHCH1(JMAb136)-Fc | see Table 2 |
| | knob chain-VH(4B9) | |
| 33 | VHCH1(JMAb136)-Fc | see Table 3 |
| | hole chain-VL(4B9) | |
| 34 | CD3 CDR-H1 | TYAMN |
| 35 | CD3 CDR-H2 | RIRSKYNNYATYYADSVKG |
| 36 | CD3 CDR-H3 | HGNFGNSYVSWFAY |
| 37 | CD3 CDR-L1 | GSSTGAVTTSNYAN |
| 38 | CD3 CDR-L2 | GTNKRAP |
| 39 | CD3 CDR-L3 | ALWYSNLWV |
| 40 | CD3 VH | EVQLLESGGGLVQPGGSLRLSCAASGFTFSTYAMNWVRQA |
| | | PGKGLEWVSRIRSKYNNYATYYADSVKGRFTISRDDSKNT |
| | | LYLQMNSLRAEDTAVYYCVRHGNFGNSYVSWFAYWGQGTL |
| | | VTVSS |
| 41 | CD3 VL | QAVVTQEPSLTVSPGGTVTLTCGSSTGAVTTSNYANWVQE |
| | | KPGQAFRGLIGGTNKRAPGTPARFSGSLLGGKAALTLSGA |
| | | QPEDEAEYYCALWYSNLWVFGGGTKLTVL |
| 42 | CEA CDR-H1 | EFGMN |
| 43 | CEA CDR-H2 | WINTKTGEATYVEEFKG |
| 44 | CEA CDR-H3 | WDFAYYVEAMDY |
| 45 | CEA CDR-L1 | KASAAVGTYVA |
| 46 | CEA CDR-L2 | SASYRKR |
| 47 | CEA CDR-L3 | HQYYTYPLFT |
| 48 | CEA VH | QVQLVQSGAEVKKPGASVKVSCKASGYTFTEFGMNWVRQA |
| | | PGQGLEWMGWINTKTGEATYVEEFKGRVTFTTDTSTSTAY |
| | | MELRSLRSDDTAVYYCARWDFAYYVEAMDYWGQGTTVTVS |
| | | S |
| 49 | CEA VL | DIQMTQSPSSLSASVGDRVTITCKASAAVGTYVAWYQQKP |
| | | GKAPKLLIYSASYRKRGVPSRFSGSGSGTDFTLTISSLQP |
| | | EDFATYYCHQYYTYPLFTFGQGTKLEIK |
| 50 | CEA CDR-H1 | DTYMH |
| | (CEACAM5) | |
| 51 | CEA CDR-H2 | RIDPANGNSKYVPKFQG |
| | (CEACAM5) | |
| 52 | CEA CDR-H3 | FGYYVSDYAMAY |
| | (CEACAM5) | |
| 53 | CEA CDR-L1 | RAGESVDIFGVGFLH |
| | (CEACAM5) | |
| 54 | CEA CDR-L2 | RASNRAT |
| | (CEACAM5) | |
| 55 | CEA-CDR-L3 | QQTNEDPYT |
| | (CEACAM5) | |
| 56 | CEA VH(CEACAM5) | QVQLVQSGAEVKKPGSSVKVSCKASGFNIKDTYMHWVRQA |
| | | PGQGLEWMGRIDPANGNSKYVPKFQGRVTITADTSTSTAY |
| | | MELSSLRSEDTAVYYCAPFGYYVSDYAMAYWGQGTLVTVS |
| | | S |
| 57 | CEA VL(CEACAM5) | EIVLTQSPATLSLSPGERATLSCRAGESVDIFGVGFLHWY |
| | | QQKPGQAPRLLIYRASNRATGIPARFSGSGSGTDFTLTIS |
| | | SLEPEDFAVYYCQQTNEDPYTFGQGTKLEIK |
| 58 | Light chain | DIQMTQSPSSLSASVGDRVTITCKASAAVGTYVAWYQQKP |
| | _{″}CEA_{2F1}″ | GKAPKLLIYSASYRKRGVPSRFSGSGSGTDFTLTISSLQP |
| | (CEA TCB) | EDFATYYCHQYYTYPLFTFGQGTKLEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |
| 59 | Light Chain humanized | QAVVTQEPSLTVSPGGTVTLTCGSSTGAVTTSNYANWVQE |
| | CD3_{CH2527}(Crossfab, VL- | KPGQAFRGLIGGTNKRAPGTPARFSGSLLGGKAALTLSGA |
| | CH1) | QPEDEAEYYCALWYSNLWVFGGGTKLTVLSSASTKGPSVF |
| | (CEA TCB) | PLAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSC |
| 60 | CEA_{CH1A1A} _{98/99}- | QVQLVQSGAEVKKPGASVKVSCKASGYTFTEFGMNWVRQA |
| | humanized CD3_{CH2527} | PGQGLEWMGWINTKTGEATYVEEFKGRVTFTTDTSTSTAY |
| | (Crossfab VH-Ck)- | MELRSLRSDDTAVYYCARWDFAYYVEAMDYWGQGTTVTVS |
| | Fc(knob) P329GLALA | SASTKGPSVFPLAPSSKSTSGGTAALGCLVKDYFPEPVTV |
| | (CEA TCB) | SWNSGALTSGVHTFPAVLQSSGLYSLSSVVTVPSSSLGTQ |
| | | TYICNVNHKPSNTKVDKKVEPKSCDGGGGSGGGGSEVQLL |
| | | ESGGGLVQPGGSLRLSCAASGFTFSTYAMNWVRQAPGKGL |
| | | EWVSRIRSKYNNYATYYADSVKGRFTISRDDSKNTLYLQM |
| | | NSLRAEDTAVYYCVRHGNFGNSYVSWFAYWGQGTLVTVSS |
| | | ASVAAPSVFIFPPSDEQLKSGTASVVCLLNNFYPREAKVQ |
| | | WKVDNALQSGNSQESVTEQDSKDSTYSLSSTLTLSKADYE |
| | | KHKVYACEVTHQGLSSPVTKSFNRGECDKTHTCPPCPAPE |
| | | AAGGPSVFLFPPKPKDTLMISRTPEVTCVVVDVSHEDPEV |
| | | KFNWYVDGVEVHNAKTKPREEQYNSTYRVVSVLTVLHQDW |
| | | LNGKEYKCKVSNKALGAPIEKTISKAKGQPREPQVYTLPP |
| | | CRDELTKNQVSLWCLVKGFYPSDIAVEWESNGQPENNYKT |
| | | TPPVLDSDGSFFLYSKLTVDKSRWQQGNVFSCSVMHEALH |
| | | NHYTQKSLSLSPGK |
| 61 | CEA_{CH1A1A} _{98/99}(VH-CH1)- | QVQLVQSGAEVKKPGASVKVSCKASGYTFTEFGMNWVRQA |
| | Fc(hole) P329GLALA | PGQGLEWMGWINTKTGEATYVEEFKGRVTFTTDTSTSTAY |
| | (CEA TCB) | MELRSLRSDDTAVYYCARWDFAYYVEAMDYWGQGTTVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKKVEPKSCDKTHTCPPCPAPEAAG |
| | | GPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALGAPIEKTISKAKGQPREPQVCTLPPSRD |
| | | ELTKNQVSLSCAVKGFYPSDIAVEWESNGQPENNYKTTPP |
| | | VLDSDGSFFLVSKLTVDKSRWQQGNVFSCSVMHEALHNHY |
| | | TQKSLSLSPGK |
| 62 | CD3 VH-CL (CEACAM5 | EVQLLESGGGLVQPGGSLRLSCAASGFTFSTYAMNWVRQA |
| | TCB) | PGKGLEWVSRIRSKYNNYATYYADSVKGRFTISRDDSKNT |
| | | LYLQMNSLRAEDTAVYYCVRHGNFGNSYVSWFAYWGQGTL |
| | | VTVSSASVAAPSVFIFPPSDEQLKSGTASVVCLLNNFYPR |
| | | EAKVQWKVDNALQSGNSQESVTEQDSKDSTYSLSSTLTLS |
| | | KADYEKHKVYACEVTHQGLSSPVTKSFNRGEC |
| 63 | humanized CEA VH- | QVQLVQSGAEVKKPGSSVKVSCKASGFNIKDTYMHWVRQA |
| | CH1(EE)-Fc(hole, P329G | PGQGLEWMGRIDPANGNSKYVPKFQGRVTITADTSTSTAY |
| | LALA) | MELSSLRSEDTAVYYCAPFGYYVSDYAMAYWGQGTLVTVS |
| | (CEACAM5 TCB) | SASTKGPSVFPLAPSSKSTSGGTAALGCLVEDYFPEPVTV |
| | | SWNSGALTSGVHTFPAVLQSSGLYSLSSVVTVPSSSLGTQ |
| | | TYICNVNHKPSNTKVDEKVEPKSCDKTHTCPPCPAPEAAG |
| | | GPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALGAPIEKTISKAKGQPREPQVCTLPPSRD |
| | | ELTKNQVSLSCAVKGFYPSDIAVEWESNGQPENNYKTTPP |
| | | VLDSDGSFFLVSKLTVDKSRWQQGNVFSCSVMHEALHNHY |
| | | TQKSLSLSP |
| 64 | humanized CEA VH- | QVQLVQSGAEVKKPGSSVKVSCKASGFNIKDTYMHWVRQA |
| | CH1(EE)-CD3 VL-CH1-Fc | PGQGLEWMGRIDPANGNSKYVPKFQGRVTITADTSTSTAY |
| | (knob, P329G LALA) | MELSSLRSEDTAVYYCAPFGYYVSDYAMAYWGQGTLVTVS |
| | (CEACAM5 TCB) | SASTKGPSVFPLAPSSKSTSGGTAALGCLVEDYFPEPVTV |
| | | SWNSGALTSGVHTFPAVLQSSGLYSLSSVVTVPSSSLGTQ |
| | | TYICNVNHKPSNTKVDEKVEPKSCDGGGGSGGGGSQAVVT |
| | | QEPSLTVSPGGTVTLTCGSSTGAVTTSNYANWVQEKPGQA |
| | | FRGLIGGTNKRAPGTPARFSGSLLGGKAALTLSGAQPEDE |
| | | AEYYCALWYSNLWVFGGGTKLTVLSSASTKGPSVFPLAPS |
| | | SKSTSGGTAALGCLVKDYFPEPVTVSWNSGALTSGVHTFP |
| | | AVLQSSGLYSLSSVVTVPSSSLGTQTYICNVNHKPSNTKV |
| | | DKKVEPKSCDKTHTCPPCPAPEAAGGPSVFLFPPKPKDTL |
| | | MISRTPEVTCVVVDVSHEDPEVKFNWYVDGVEVHNAKTKP |
| | | REEQYNSTYRVVSVLTVLHQDWLNGKEYKCKVSNKALGAP |
| | | IEKTISKAKGQPREPQVYTLPPCRDELTKNQVSLWCLVKG |
| | | FYPSDIAVEWESNGQPENNYKTTPPVLDSDGSFFLYSKLT |
| | | VDKSRWQQGNVFSCSVMHEALHNHYTQKSLSLSP |
| 65 | humanized CEA VL-CL(RK) | EIVLTQSPATLSLSPGERATLSCRAGESVDIFGVGFLHWY |
| | (CEACAM5 TCB) | QQKPGQAPRLLIYRASNRATGIPARFSGSGSGTDFTLTIS |
| | | SLEPEDFAVYYCQQTNEDPYTFGQGTKLEIKRTVAAPSVF |
| | | IFPPSDRKLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| 66 | Fc hole chain-VL(4B9) | see Table 2 |
| 67 | VHCH1(DP47) Fc hole | see Table 4 |
| | chain | |
| 68 | VLCL(DP47) Light chain | see Table 4 |
| 69 | VHCH1(JMAb136)-Fc | see Table 5 |
| | knob chain-VH(DP47) | |
| 70 | VHCH1(JMAb136)-Fc | see Table 5 |
| | hole chain-VL(DP47) | |
| 71 | Murine ICOSL Linker | see Table 7 |
| | muIgG1 Fc(DAPG KK) | |
| | 4GS linker FAP(28H1) | |
| | VH | |
| 72 | Murine ICOSL linker | see Table 7 |
| | muIgG1 Fc(DAPG DD) | |
| | 4GS linker FAP(28H1) | |
| | VL | |
| 73 | Murine ICOSL Linker | see Table 8 |
| | muIgG1 Fc(DAPG KK) | |
| | G4S linker DP47 VH | |
| 74 | Murine ICOSL linker | see Table 8 |
| | muIgG1 Fc(DAPG DD) | |
| | 4GS linker DP47 VL | |
| 75 | VHCH1(CH1A1A 98/99 | see Table 10 |
| | 2F1)-Fc(KK) DAPG chain | |
| 76 | VLCL(CH1A1A 98/99 | see Table 10 |
| | 2F1) Light chain | |
| 77 | VHCL VHCH1(2C11- | see Table 10 |
| | CH1A1A 98/99 2F1)- | |
| | Fc(DD) DAPG chain | |
| 78 | VLCH1(2C11) | see Table 10 |
| | Light chain | |
| 79 | human FAP | UniProt accession no. Q12884 |
| 80 | His-tagged human FAP | RPSRVHNSEENTMRALTLKDILNGTFSYKTFFPNWISGQE |
| | ECD | YLHQSADNNIVLYNIETGQSYTILSNRTMKSVNASNYGLS |
| | | PDRQFVYLESDYSKLWRYSYTATYYIYDLSNGEFVRGNEL |
| | | PRPIQYLCWSPVGSKLAYVYQNNIYLKQRPGDPPFQITFN |
| | | GRENKIFNGIPDWVYEEEMLATKYALWWSPNGKFLAYAEF |
| | | NDTDIPVIAYSYYGDEQYPRTINIPYPKAGAKNPVVRIFI |
| | | IDTTYPAYVGPQEVPVPAMIASSDYYFSWLTWVTDERVCL |
| | | QWLKRVQNVSVLSICDFREDWQTWDCPKTQEHIEESRTGW |
| | | AGGFFVSTPVFSYDAISYYKIFSDKDGYKHIHYIKDTVEN |
| | | AIQITSGKWEAINIFRVTQDSLFYSSNEFEEYPGRRNIYR |
| | | ISIGSYPPSKKCVTCHLRKERCQYYTASFSDYAKYYALVC |
| | | YGPGIPISTLHDGRTDQEIKILEENKELENALKNIQLPKE |
| | | EIKKLEVDEITLWYKMILPPQFDRSKKYPLLIQVYGGPCS |
| | | QSVRSVFAVNWISYLASKEGMVIALVDGRGTAFQGDKLLY |
| | | AVYRKLGVYEVEDQITAVRKFIEMGFIDEKRIAIWGWSYG |
| | | GYVSSLALASGTGLFKCGIAVAPVSSWEYYASVYTERFMG |
| | | LPTKDDNLEHYKNSTVMARAEYFRNVDYLLIHGTADDNVH |
| | | FQNSAQIAKALVNAQVDFQAMWYSDQNHGLSGLSTNHLYT |
| | | HMTHFLKQCFSLSDGKKKKKKGHHHHHH |
| 81 | mouse FAP | UniProt accession no. P97321 |
| 82 | His-tagged mouse FAP | RPSRVYKPEGNTKRALTLKDILNGTFSYKTYFPNWISEQE |
| | ECD | YLHQSEDDNIVFYNIETRESYIILSNSTMKSVNATDYGLS |
| | | PDRQFVYLESDYSKLWRYSYTATYYIYDLQNGEFVRGYEL |
| | | PRPIQYLCWSPVGSKLAYVYQNNIYLKQRPGDPPFQITYT |
| | | GRENRIFNGIPDWVYEEEMLATKYALWWSPDGKFLAYVEF |
| | | NDSDIPIIAYSYYGDGQYPRTINIPYPKAGAKNPVVRVFI |
| | | VDTTYPHHVGPMEVPVPEMIASSDYYFSWLTWVSSERVCL |
| | | QWLKRVQNVSVLSICDFREDWHAWECPKNQEHVEESRTGW |
| | | AGGFFVSTPAFSQDATSYYKIFSDKDGYKHIHYIKDTVEN |
| | | AIQITSGKWEAIYIFRVTQDSLFYSSNEFEGYPGRRNIYR |
| | | ISIGNSPPSKKCVTCHLRKERCQYYTASFSYKAKYYALVC |
| | | YGPGLPISTLHDGRTDQEIQVLEENKELENSLRNIQLPKV |
| | | EIKKLKDGGLTFWYKMILPPQFDRSKKYPLLIQVYGGPCS |
| | | QSVKSVFAVNWITYLASKEGIVIALVDGRGTAFQGDKFLH |
| | | AVYRKLGVYEVEDQLTAVRKFIEMGFIDEERIAIWGWSYG |
| | | GYVSSLALASGTGLFKCGIAVAPVSSWEYYASIYSERFMG |
| | | LPTKDDNLEHYKNSTVMARAEYFRNVDYLLIHGTADDNVH |
| | | FQNSAQIAKALVNAQVDFQAMWYSDQNHGILSGRSQNHLY |
| | | THMTHFLKQCFSLSDGKKKKKKGHHHHHH |
| 83 | His-tagged cynomolgus | RPPRVHNSEENTMRALTLKDILNGTFSYKTFFPNWISGQE |
| | FAP ECD | YLHQSADNNIVLYNIETGQSYTILSNRTMKSVNASNYGLS |
| | | PDRQFVYLESDYSKLWRYSYTATYYIYDLSNGEFVRGNEL |
| | | PRPIQYLCWSPVGSKLAYVYQNNIYLKQRPGDPPFQITFN |
| | | GRENKIFNGIPDWVYEEEMLATKYALWWSPNGKFLAYAEF |
| | | NDTDIPVIAYSYYGDEQYPRTINIPYPKAGAKNPFVRIFI |
| | | IDTTYPAYVGPQEVPVPAMIASSDYYFSWLTWVTDERVCL |
| | | QWLKRVQNVSVLSICDFREDWQTWDCPKTQEHIEESRTGW |
| | | AGGFFVSTPVFSYDAISYYKIFSDKDGYKHIHYIKDTVEN |
| | | AIQITSGKWEAINIFRVTQDSLFYSSNEFEDYPGRRNIYR |
| | | ISIGSYPPSKKCVTCHLRKERCQYYTASFSDYAKYYALVC |
| | | YGPGIPISTLHDGRTDQEIKILEENKELENALKNIQLPKE |
| | | EIKKLEVDEITLWYKMILPPQFDRSKKYPLLIQVYGGPCS |
| | | QSVRSVFAVNWISYLASKEGMVIALVDGRGTAFQGDKLLY |
| | | AVYRKLGVYEVEDQITAVRKFIEMGFIDEKRIAIWGWSYG |
| | | GYVSSLALASGTGLFKCGIAVAPVSSWEYYASVYTERFMG |
| | | LPTKDDNLEHYKNSTVMARAEYFRNVDYLLIHGTADDNVH |
| | | FQNSAQIAKALVNAQVDFQAMWYSDQNHGLSGLSTNHLYT |
| | | HMTHFLKQCFSLSDGKKKKKKGHHHHHH |
| 84 | human CEA | UniProt accession no. P06731 |
| 85 | human FolR1 | UniProt accession no. P15328 |
| 86 | murine FolR1 | UniProt accession no. P35846 |
| 87 | cynomolgus FolR1 | UniProt accession no. G7PR14 |
| 88 | human MCSP | UniProt accession no. Q6UVK1 |
| 89 | human EGFR | UniProt accession no. P00533 |
| 90 | human HER2 | Uniprot accession no. P04626 |
| 91 | p95 HER2 | MPIWKFPDEEGACQPCPINCTHSCVDLDDKGCPAEQRASP |
| | | LTSIISAVVGILLVVVLGVVFGILIKRRQQKIRKYTMRRL |
| | | LQETELVEPLTPSGAMPNQAQMRILKETELRKVKVLGSGA |
| | | FGTVYKGIWIPDGENVKIPVAIKVLRENTSPKANKEILDE |
| | | AYVMAGVGSPYVSRLLGICLTSTVQLVTQLMPYGCLLDHV |
| | | RENRGRLGSQDLLNWCMQIAKGMSYLEDVRLVHRDLAARN |
| | | VLVKSPNHVKITDFGLARLLDIDETEYHADGGKVPIKWMA |
| | | LESILRRRFTHQSDVWSYGVTVWELMTFGAKPYDGIPARE |
| | | IPDLLEKGERLPQPPICTIDVYMIMVKCWMIDSECRPRFR |
| | | ELVSEFSRMARDPQRFVVIQNEDLGPASPLDSTFYRSLLE |
| | | DDDMGDLVDAEEYLVPQQGFFCPDPAPGAGGMVHHRHRSS |
| | | STRSGGGDLTLGLEPSEEEAPRSPLAPSEGAGSDVFDGDL |
| | | GMGAAKGLQSLPTHDPSPLQRYSEDPTVPLPSETDGYVAP |
| | | LTCSPQPEYVNQPDVRPQPPSPREGPLPAARPAGATLERP |
| | | KTLSPGKNGVVKDVFAFGGAVENPEYLTPQGGAAPQPHPP |
| | | PAFSPAFDNLYYWDQDPPERGAPPSTFKGTPTAENPEYLG |
| | | LDVPV |
| 92 | Peptide linker(G4S) | GGGGS |
| 93 | Peptide linker(G4S)₂ | GGGGSGGGGS |
| 94 | Peptide linker(G4S)₂ | SGGGGSGGGG |
| 95 | Peptide linker G4(SG4)₂ | GGGGSGGGGSGGGG |
| 96 | peptide linker | GSPGSSSSGS |
| 97 | (G4S)₃ peptide linker | GGGGSGGGGSGGGGS₃ |
| 98 | (G4S)₄ peptide linker | GGGGSGGGGSGGGGSGGGGS |
| 99 | peptide linker | GSGSGSGS |
| 100 | peptide linker | GSGSGNGS |
| 101 | peptide linker | GGSGSGSG |
| 102 | peptide linker | GGSGSG |
| 103 | peptide linker | GGSG |
| 104 | peptide linker | GGSGNGSG |
| 105 | peptide linker | GGNGSGSG |
| 106 | peptide linker | GGNGSG |
| 107 | human PD-L1 | UniProt accession no. Q9NZQ7 |
| 108 | human PD-1 | UniProt accession no. Q15116 |
| 109 | VH(PD-L1) | EVQLVESGGGLVQPGGSLRLSCAASGFTFSDSWIHWVRQA |
| | | PGKGLEWVAWISPYGGSTYYADSVKGRFTISADTSKNTAY |
| | | LQMNSLRAEDTAVYYCARRHWPGGFDYWGQGTLVTVSS |
| 110 | VL(PD-L1) | DIQMTQSPSSLSASVGDRVTITCRASQDVSTAVAWYQQKP |
| | | GKAPKLLIYSASFLYSGVPSRFSGSGSGTDFTLTISSLQP |
| | | EDFATYYCQQYLYHPATFGQGTKVEIK |
| 111 | VH(PD-L1)2 | EVQLVESGGGLVQPGGSLRLSCAASGFTFSRYWMSWVRQA |
| | | PGKGLEWVANIKQDGSEKYYVDSVKGRFTISRDNAKNSLY |
| | | LQMNSLRAEDTAVYYCAREGGWFGELAFDYWGQGTLVTVS |
| | | S |
| 112 | VL(PD-L1)2 | EIVLTQSPGTLSLSPGERATLSCRASQRVSSSYLAWYQQK |
| | | PGQAPRLLIYDASSRATGIPDRFSGSGSGTDFTLTISRLE |
| | | PEDFAVYYCQQYGSLPWTFGQGTKVEIK |
| 113 | VH(PD-1) | QVQLVQSGVEVKKPGASVKVSCKASGYTFTNYYMYWVRQA |
| | | PGQGLEWMGGINPSNGGTNFNEKFKNRVTLTTDSSTTTAY |
| | | MELKSLQFDDTAVYYCARRDYRFDMGFDYWGQGTTVTVSS |
| 114 | VL(PD-1) | EIVLTQSPATLSLSPGERATLSCRASKGVSTSGYSYLHWY |
| | | QQKPGQAPRLLIYLASYLESGVPARFSGSGSGTDFTLTIS |
| | | SLEPEDFAVYYCQHSRDLPLTFGGGTKVEIK |
| 115 | VH(PD-1)2 | QVQLVESGGGVVQPGRSLRLDCKASGITFSNSGMHWVRQA |
| | | PGKGLEWVAVIWYDGSKRYYADSVKGRFTISRDNSKNTLF |
| | | LQMNSLRAEDTAVYYCATNDDYWGQGTLVTVSS |
| 116 | VL(PD-1)2 | EIVLTQSPATLSLSPGERATLSCRASQSVSSYLAWYQQKP |
| | | GQAPRLLIYDASNRATGIPARFSGSGSGTDFTLTISSLEP |
| | | EDFAVYYCQQSSNWPRTFGQGTKVEIK |
| 117 | Murine ICOS | UniprotKB accession no. Q9WVS0 |
| | | MKPYFCRVFV FCFLIRLLTG EINGSADHRM |
| | | FSFHNGGVQI SCKYPETVQQ LKMRLFRERE |
| | | VLCELTKTKG SGNAVSIKNP LMCLYHLSNN |
| | | SVSFFLNNPD SSQGSYYFCS LSIFDPPPFQ |
| | | ERNLSGGYLH IYESQLCCQL KLWLPVGCAA |
| | | FVVVLLFGCI LIIWFSKKKY GSSVHDPNSE |
| | | YMFMAAVNTN KKSRLAGVTS |
| 118 | Murine ICOS(21-144) ECD | EINGSADHRM FSFHNGGVQI SCKYPETVQQ |
| | | LKMRLFRERE VLCELTKTKG SGNAVSIKNP |
| | | MLCLYHLSNN SVSFFLNNPD SSQGSYYFCS |
| | | LSIFDPPPFQ ERNLSGGYLH IYESQLCCQL KLWL |
| 119 | Nucleotide sequence | See Table 16 |
| | murine ICOS antigen Fc | |
| | hole chain | |
| 120 | Nucleotide sequence | See Table 16 |
| | murine ICOS antigen Fc | |
| | knob chain | |
| 121 | murine ICOS antigen Fc | See Table 16 |
| | hole chain | |
| 122 | murine ICOS antigen Fc | See Table 16 |
| | knob chain | |
| 123 | Murine ICOS CDR-H1 | GYSFTSYWIG |
| 124 | Murine ICOS CDR-H2 | IIYPGDSDTRYSPSFQG |
| 125 | Murine ICOS CDR-H3 | SSGPYGLYLDY |
| 126 | Murine ICOS CDR-L1 | RSSQSLLHSNGYNYLD |
| 127 | Murine ICOS CDR-L2 | LGSNRAS |
| 128 | Murine ICOS CDR-L3 | MQALWTPTT |
| 129 | Murine ICOS(16E09) VH | EVQLVQSGAEVKKPGESLKISCKGSGYSFTSYWIGWVRQM |
| | | PGKGLEWMGIIYPGDSDTRYSPSFQGQVTISADKSISTAY |
| | | LQWSSLKASDTAMYYCARSSGPYGLYLDYWGQGTLVTVSS |
| 130 | Murine ICOS(16E09) VL | DIVMTQSPLSLPVTPGEPASISCRSSQSLLHSNGYNYLDW |
| | | YLQKPGQSPQLLIYLGSNRASGVPDRFSGSGSGTDFTLKI |
| | | SRVEAEDVGVYYCMQALWTPTTFGQGTKVEIK |
| 131 | Murine ICOS(16E09) | See Table 19 |
| | P329GLALA human IgG1 | |
| | light chain | |
| 132 | Murine ICOS(16E09) | See Table 19 |
| | P329GLALA human IgG1 | |
| | heavy chain | |
| 133 | murine ICOS Fc knob Avi- | See Table 21 |
| | tag | |
| 134 | murine ICOS Fc hole | See Table 21 |
| 135 | VHCH1(16E09) Fc | See Table 23 |
| | DAPG DD heavy chain- | |
| | VH(28H1) | |
| 136 | VHCH1(16E09) Fc | See Table 23 |
| | DAPG KK heavy chain- | |
| | VL(28H1) | |
| 137 | VHCH1(16E09) Fc | See Table 24 |
| | DAPG DD heavy chain | |
| 138 | VHCH1(28H1) Fc DAPG | See Table 24 |
| | KK heavy chain | |
| 139 | VLCL(28H1)-light chain | See Table 24 |
| 140 | VHCH1(16E09) Fc | See Table 25 |
| | DAPG DD heavy chain- | |
| | VH(DP47) | |
| 141 | VHCH1(16E09) Fc | See Table 25 |
| | DAPG KK heavy chain- | |
| | VL(DP47) | |
| 142 | VHCH1(16E09) Fc | See Table 26 |
| | DAPG DD heavy chain | |
| 143 | VHCH1(DP47) Fc DAPG | See Table 26 |
| | KK heavy chain | |
| 144 | Murine VLCL(DP47)- | See Table 26 |
| | light chain | |
| 145 | CEA(MEDI-565)-CDR- | SYWMH |
| | H1 | |
| 146 | CEA(MEDI-565)-CDR- | FIRNKANGGTTEYAAS |
| | H2 | |
| 147 | CEA(MEDI-565)-CDR- | DRGLRFYFDY |
| | H3 | |
| 148 | CEA(MEDI-565)-CDR- | TLRRGINVGAYSIY |
| | L1 | |
| 149 | CEA(MEDI-565)-CDR- | YKSDSDKQQGS |
| | L2 | |
| 150 | CEA(MEDI-565)-CDR- | MIWHSGASAV |
| | L3 | |
| 151 | CEA(MEDI-565) VH | EVQLVESGGGLVQPGRSLRLSCAASGFTVSSYWMHWVRQA |
| | | PGKGLEWVGFIRNKANGGTTEYAASVKGRFTISRDDSKNT |
| | | LYLQMNSLRAEDTAVYYCARDRGLRFYFDYWGQGTTVTVS |
| | | S |
| 152 | CEA(MEDI-565) VL | QAVLTQPASLSASPGASASLTCTLRRGINVGAYSIYWYQQ |
| | | KPGSPPQYLLRYKSDSDKQQGSGVSSRFSASKDASANAGI |
| | | LLISGLQSEDEADYYCMIWHSGASAVFGGGTKLTVL |
| 153 | VHCH1(JMAb136)-Fc | See Table 28 |
| | knob chain-VH(MEDI- | |
| | 565) | |
| 154 | VHCH1(JMAb136)-Fc | See Table 28 |
| | hole chain-VL(MEDI- | |
| | 565) | |
| 155 | VHCH1(JMAb136)-Fc | See Table 29 |
| | hole chain | |
| 156 | VHCH1(MEDI-565)-Fc | See Table 29 |
| | knob chain | |
| 157 | VLCL(MEDI-565) Light | See Table 29 |
| | chain | |
| 158 | CEA(A5B7)-CDR-H1 | DYYMN |
| 159 | CEA(A5B7)-CDR-H2 | FIGNKANGYTTEYSASVKG |
| 160 | CEA(A5B7)-CDR-H3 | DRGLRFYFDY |
| 161 | CEA(A5B7)-CDR-L1 | RASSSVTYIH |
| 162 | CEA(A5B7)-CDR-L2 | ATSNLAS |
| 163 | CEA(A5B7)-CDR-L3 | QHWSSKPPT |
| 164 | CEA(A5B7) VH | EVKLVESGGGLVQPGGSLRLSCATSGFTFTDYYMNWVRQP |
| | | PGKALEWLGFIGNKANGYTTEYSASVKGRFTISRDKSQSI |
| | | LYLQMNTLRAEDSATYYCTRDRGLRFYFDYWGQGTTLTVS |
| | | S |
| 165 | CEA(A5B7) VL | QTVLSQSPAILSASPGEKVTMTCRASSSVTYIHWYQQKPG |
| | | SSPKSWIYATSNLASGVPARFSGSGSGTSYSLTISRVEAE |
| | | DAATYYCQHWSSKPPTFGGGTKLEIK |
| 166 | VHCH1(16E09) Fc | see Table 31 |
| | DAPG DD heavy chain- | |
| | VL(A5B7) | |
| 167 | VHCH1(16E09) Fc | See Table 31 |
| | DAPG KK heavy chain- | |
| | VH(A5B7) | |
| 168 | VHCH1(A5B7) Fc DAPG | see Table 32 |
| | KK heavy chain | |
| 169 | VLCL(A5B7)-light chain | see Table 32 |
| 170 | CH1 domain | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKV |
| 171 | linker | EPKSC |
| 172 | hinge region | DKTHTCPXCP with X = S or P |
| 173 | short hinge region | HTCPXCP with X = S or P |
| 174 | shortest hinge region | CPXCP with X = S or P |
| 175 | CH2 domain | APELLGGPSV FLFPPKPKDT LMISRTPEVT CVWDVSHEDP |
| | | EVKFNWYVDG VEVHNAKTKP REEQESTYRW SVLTVLHQDW |
| | | LNGKEYKCKV SNKALPAPIE KTISKAK |
| 176 | CH3 domain | GQPREPQVYT LPPSRDELTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSP |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, Md. (1991)) as defined above.

The following numbered paragraphs (paras) describe aspects of the present invention:

1. An agonistic ICOS-binding molecule comprising at least one antigen binding domain that binds to a tumor-associated antigen for use in a method for treating or delaying progression of cancer, wherein the agonistic ICOS-binding molecule comprising at least one antigen binding domain that binds to a tumor-associated antigen is used in combination with a T-cell activating anti-CD3 bispecific antibody specific for a tumor-associated antigen.

2. The agonistic ICOS-binding molecule comprising at least one antigen binding domain that binds to a tumor-associated antigen for use in a method of para 1, wherein the T-cell activating anti-CD3 bispecific antibody specific for a tumor-associated antigen is an anti-CEA/anti-CD3 bispecific antibody.

3. The agonistic ICOS-binding molecule comprising at least one antigen binding domain that binds to a tumor-associated antigen for use in a method of paras 1 or 2, wherein the agonistic ICOS-binding molecule comprising at least one antigen binding domain that binds to a tumor-associated antigen and the T-cell activating anti-CD3 bispecific antibody are administered together in a single composition or administered separately in two or more different compositions.

4. The agonistic ICOS-binding molecule comprising at least one antigen binding domain that binds to a tumor-associated antigen for use in a method of paras 1 to 3, wherein the agonistic ICOS-binding molecule comprising at least one antigen binding domain that binds to a tumor-associated antigen acts synergistically with the T-cell activating anti-CD3 bispecific antibody.

5. The agonistic ICOS-binding molecule comprising at least one antigen binding domain that binds to a tumor-associated antigen for use in a method of any one of paras 1 to 4, wherein the agonistic ICOS-binding molecule comprising at least one antigen binding domain that binds to a tumor-associated antigen comprises at least one ICOS-L or fragments thereof.

6. The agonistic ICOS-binding molecule comprising at least one antigen binding domain that binds to a tumor-associated antigen for use in a method of any one of paras 1 to 5, wherein the agonistic ICOS-binding molecule comprising at least one antigen binding domain that binds to a tumor-associated antigen comprises at least one ICOS-L comprising the amino acid sequence of SEQ ID NO: 1 or SEQ ID NO:2.

7. The agonistic ICOS-binding molecule comprising at least one antigen binding domain that binds to a tumor-associated antigen for use in a method of any one of paras 1 to 4, wherein the agonistic ICOS-binding molecule comprising at least one antigen binding domain that binds to a tumor-associated antigen comprises at least one antigen binding domain that is capable of agonistic binding to ICOS.

8. The agonistic ICOS-binding molecule comprising at least one antigen binding domain that binds to a tumor-associated antigen for use in a method of any one of paras 1 to 4, wherein the agonistic ICOS-binding molecule comprising at least one antigen binding domain that binds to a tumor-associated antigen comprises at least one antigen binding domain that is capable of agonistic binding to human ICOS comprising the amino acid sequence of SEQ ID NO:3.

9. The agonistic ICOS-binding molecule comprising at least one antigen binding domain that binds to a tumor-associated antigen for use in a method of any one of paras 1 to 8, wherein the agonistic ICOS-binding molecule comprising at least one antigen binding domain that binds to a tumor-associated antigen is an agonistic ICOS-binding molecule comprising at least one antigen binding domain that binds to Fibroblast activation protein (FAP).

10. The agonistic ICOS-binding molecule comprising at least one antigen binding domain that binds to a tumor-associated antigen for use in a method of any one of paras 1 to 9, wherein the agonistic ICOS-binding molecule comprises at least one antigen binding domain capable of specific binding to FAP comprising

(a) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}FAP) comprising (i) CDR-H1 comprising the amino acid sequence of SEQ ID NO: 12, (ii) CDR-H2 comprising the amino acid sequence of SEQ ID NO:13, and (iii) CDR-H3 comprising the amino acid sequence of SEQ ID NO: 14, and a light chain variable region (V_{L}FAP) comprising (iv) CDR-L1 comprising the amino acid sequence of SEQ ID NO: 15, (v) CDR-L2 comprising the amino acid sequence of SEQ ID NO: 16, and (vi) CDR-L3 comprising the amino acid sequence of SEQ ID NO: 17.

11. The agonistic ICOS-binding molecule comprising at least one antigen binding domain that binds to a tumor-associated antigen for use in a method of any one of paras 1 to 10, wherein the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11 or wherein the antigen binding domain capable of specific binding to FAP comprises a heavy chain variable region (V_{H}FAP) comprising an amino acid sequence of SEQ ID NO: 18 and a light chain variable region (V_{L}FAP) comprising an amino acid sequence of SEQ ID NO: 19.

12. The agonistic ICOS-binding molecule comprising at least one antigen binding domain that binds to a tumor-associated antigen for use in a method of any one of paras 1 to 4 or 7 to 11, wherein the agonistic ICOS-binding molecule comprising at least one antigen binding domain that binds to a tumor-associated antigen comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25.

13. The agonistic ICOS-binding molecule comprising at least one antigen binding domain that binds to a tumor-associated antigen for use in a method of any one of paras 1 to 4 or 7 to 12, wherein the agonistic ICOS-binding molecule comprising at least one antigen binding domain that binds to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27.

14. The agonistic ICOS-binding molecule comprising at least one antigen binding domain that binds to a tumor-associated antigen for use in a method of any one of paras 1 to 13, wherein the agonistic ICOS-binding molecule comprising at least one antigen binding domain that binds to a tumor-associated antigen comprises an IgG Fc domain, specifically an IgG1 Fc domain or an IgG4 Fc domain.

15. The agonistic ICOS-binding molecule comprising at least one antigen binding domain that binds to a tumor-associated antigen for use in a method of any one of paras 1 to 14, wherein the agonistic ICOS-binding molecule comprising at least one antigen binding domain that binds to a tumor-associated antigen comprises a Fc domain that comprises one or more amino acid substitution that reduces binding to an Fc receptor and/or effector function.

16. The agonistic ICOS-binding molecule comprising at least one antigen binding domain that binds to a tumor-associated antigen for use in a method of any one of paras 1 to 4 or 7 to 15, wherein the agonistic ICOS-binding molecule comprises a first heavy chain comprising an amino acid sequence of SEQ ID NO:28, a first light chain comprising an amino acid sequence of SEQ ID NO:29, a second heavy chain comprising an amino acid sequence of SEQ ID NO:30, and a second light chain comprising an amino acid sequence of SEQ ID NO:31 or wherein the agonistic ICOS-binding molecule comprises a first heavy chain comprising an amino acid sequence of SEQ ID NO:32, a second heavy chain comprising an amino acid sequence of SEQ ID NO:66, and one light chain comprising the amino acid sequence of SEQ ID NO:29.

17. The agonistic ICOS-binding molecule comprising at least one antigen binding domain that binds to a tumor-associated antigen for use in a method of any one of paras 1 to 4 or 7 to 15, wherein the agonistic ICOS-binding molecule comprises monovalent binding to a tumor associated target and bivalent binding to ICOS.

18. The agonistic ICOS-binding molecule comprising at least one antigen binding domain that binds to a tumor-associated antigen for use in a method of any one of paras 1 to 4 or 7 to 15 or 17, wherein the agonistic ICOS-binding molecule comprises a first heavy chain comprising an amino acid sequence of SEQ ID NO:32, a second heavy chain comprising an amino acid sequence of SEQ ID NO:33, and a two light chains comprising an amino acid sequence of SEQ ID NO:29.

19. The agonistic ICOS-binding molecule comprising at least one antigen binding domain that binds to a tumor-associated antigen for use in a method of any one of paras 1 to 18, wherein the T-cell activating anti-CD3 bispecific antibody is an anti-CEA/anti-CD3 bispecific antibody.

20. The agonistic ICOS-binding molecule comprising at least one antigen binding domain that binds to a tumor-associated antigen for use in a method of any one of paras 1 to 19, wherein the T-cell activating anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

21. The agonistic ICOS-binding molecule comprising at least one antigen binding domain that binds to a tumor-associated antigen for use in a method of any one of paras 1 to 20, wherein the T-cell activating anti-CD3 bispecific antibody comprises a first antigen binding domain comprising a heavy chain variable region (V_{H}CD3) comprising (i) CDR-H1 comprising the amino acid sequence of SEQ ID NO:34, (ii) CDR-H2 comprising the amino acid sequence of SEQ ID NO:35, and (iii) CDR-H3 comprising the amino acid sequence of SEQ ID NO:36, and a light chain variable region (V_{L}CD3) comprising (iv) CDR-L1 comprising the amino acid sequence of SEQ ID NO:37, (v) CDR-L2 comprising the amino acid sequence of SEQ ID NO:38, and (vi) CDR-L3 comprising the amino acid sequence of SEQ ID NO:39.

22. The agonistic ICOS-binding molecule comprising at least one antigen binding domain that binds to a tumor-associated antigen for use in a method of any one of paras 1 to 21, wherein the T-cell activating anti-CD3 bispecific antibody comprises a first antigen binding domain comprising a heavy chain variable region (V_{H}CD3) comprising the amino acid sequence of SEQ ID NO:40 and/or a light chain variable region (V_{L}CD3) comprising the amino acid sequence of SEQ ID NO:41.

23. The agonistic ICOS-binding molecule comprising at least one antigen binding domain that binds to a tumor-associated antigen for use in a method of any one of paras 1 to 22, wherein the T-cell activating anti-CD3 bispecific antibody comprises a second antigen binding domain comprising

(a) a heavy chain variable region (V_{H}CEA) comprising (i) CDR-H1 comprising the amino acid sequence of SEQ ID NO:42, (ii) CDR-H2 comprising the amino acid sequence of SEQ ID NO:43, and (iii) CDR-H3 comprising the amino acid sequence of SEQ ID NO:44, and a light chain variable region (V_{L}CEA) comprising (iv) CDR-L1 comprising the amino acid sequence of SEQ ID NO:45, (v) CDR-L2 comprising the amino acid sequence of SEQ ID NO:46, and (vi) CDR-L3 comprising the amino acid sequence of SEQ ID NO:47, or
(b) a heavy chain variable region (V_{H}CEA) comprising (i) CDR-H1 comprising the amino acid sequence of SEQ ID NO:50, (ii) CDR-H2 comprising the amino acid sequence of SEQ ID NO:51, and (iii) CDR-H3 comprising the amino acid sequence of SEQ ID NO:52, and a light chain variable region (V_{L}CEA) comprising (iv) CDR-L1 comprising the amino acid sequence of SEQ ID NO:53, (v) CDR-L2 comprising the amino acid sequence of SEQ ID NO:54, and (vi) CDR-L3 comprising the amino acid sequence of SEQ ID NO:55.

24. The agonistic ICOS-binding molecule comprising at least one antigen binding domain that binds to a tumor-associated antigen for use in a method of any one of paras 1 to 23, wherein the T-cell activating anti-CD3 bispecific antibody comprises a second antigen binding domain comprising a heavy chain variable region (V_{H}CEA) comprising the amino acid sequence of SEQ ID NO:48 and/or a light chain variable region (V_{L}CEA) comprising the amino acid sequence of SEQ ID NO:49 or a second antigen binding domain comprising a heavy chain variable region (V_{H}CEA) comprising the amino acid sequence of SEQ ID NO:56 and/or a light chain variable region (V_{L}CEA) comprising the amino acid sequence of SEQ ID NO:57.

25. The agonistic ICOS-binding molecule comprising at least one antigen binding domain that binds to a tumor-associated antigen for use in a method of any one of paras 1 to 24, wherein the anti-CEA/anti-CD3 bispecific antibody comprises a third antigen binding domain that binds to CEA.

26. The agonistic ICOS-binding molecule comprising at least one antigen binding domain that binds to a tumor-associated antigen for use in a method of any one of paras 1 to 25, wherein the T-cell activating anti-CD3 bispecific antibody comprises an Fc domain comprising one or more amino acid substitutions that reduce binding to an Fc receptor and/or effector function.

27. The agonistic ICOS-binding molecule comprising at least one antigen binding domain that binds to a tumor-associated antigen for use in a method of any one of paras 1 to 25, wherein the T-cell activating anti-CD3 bispecific antibody comprises (a) the amino acid sequences of SEQ ID NO:58, SEQ ID NO:59, SEQ ID NO:60 and SEQ ID NO:61, or (b) the amino acid sequences of SEQ ID NO:62, SEQ ID NO:63, SEQ ID NO:64 and SEQ ID NO:65.

28. The agonistic ICOS-binding molecule comprising at least one antigen binding domain that binds to a tumor-associated antigen for use in a method of any one of paras 1 to 27, wherein the agonistic ICOS-binding molecule comprising at least one antigen binding domain that binds to a tumor-associated antigen is used in combination with a T-cell activating anti-CD3 bispecific antibody specific for a tumor-associated antigen and in combination with an agent blocking PD-L1/PD-1 interaction.

29. The agonistic ICOS-binding molecule comprising at least one antigen binding domain that binds to a tumor-associated antigen for use in a method of para 28, wherein the agent blocking PD-L1/PD-1 interaction is a anti-PD-L1 antibody or an anti-PD1 antibody.

30. A pharmaceutical product comprising (A) a first composition comprising as active ingredient an agonistic ICOS-binding molecule comprising at least one antigen binding domain that binds to a tumor-associated antigen and a pharmaceutically acceptable excipient; and (B) a second composition comprising a T-cell activating anti-CD3 bispecific antibody specific for a tumor-associated antigen and a pharmaceutically acceptable excipient, for use in the combined, sequential or simultaneous, treatment of a disease, in particular cancer.

31. A pharmaceutical composition comprising an agonistic ICOS-binding molecule comprising at least one antigen binding domain that binds to a tumor-associated antigen and a T-cell activating anti-CD3 bispecific antibody specific for a tumor-associated antigen and pharmaceutically acceptable excipients.

32. The pharmaceutical composition of para 31 for use in the treatment of solid tumors.

33. An agonistic ICOS-binding molecule comprising at least one antigen binding domain that binds to a tumor-associated antigen, wherein the tumor-associated antigen is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epi dermal growth factor receptor 2 (HER2) and p95HER2.

34. The agonistic ICOS-binding molecule comprising at least one antigen binding domain that binds to a tumor-associated antigen of para 33, wherein the tumor-associated antigen is FAP.

35. The agonistic ICOS-binding molecule comprising at least one antigen binding domain that binds to a tumor-associated antigen of paras 33 or 34, wherein the agonistic ICOS-binding molecule comprises at least one antigen binding domain capable of specific binding to FAP comprising

(a) a heavy chain variable region (V_{H}FAP) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}FAP) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}FAP) comprising (i) CDR-H1 comprising the amino acid sequence of SEQ ID NO: 12, (ii) CDR-H2 comprising the amino acid sequence of SEQ ID NO: 13, and (iii) CDR-H3 comprising the amino acid sequence of SEQ ID NO: 14, and a light chain variable region (V_{L}FAP) comprising (iv) CDR-L1 comprising the amino acid sequence of SEQ ID NO: 15, (v) CDR-L2 comprising the amino acid sequence of SEQ ID NO: 16, and (vi) CDR-L3 comprising the amino acid sequence of SEQ ID NO: 17.

36. The agonistic ICOS-binding molecule comprising at least one antigen binding domain that binds to a tumor-associated antigen of any one of paras 33 to 35, wherein the agonistic ICOS-binding molecule comprises at least one antigen binding domain capable of specific binding to FAP comprising a heavy chain variable region (V_{H}FAP) comprising an amino acid sequence of SEQ ID NO: 10 and a light chain variable region (V_{L}FAP) comprising an amino acid sequence of SEQ ID NO: 11 or wherein the antigen binding domain capable of specific binding to FAP comprises a heavy chain variable region (V_{H}FAP) comprising an amino acid sequence of SEQ ID NO: 18 and a light chain variable region (V_{L}FAP) comprising an amino acid sequence of SEQ ID NO: 19.

37. The agonistic ICOS-binding molecule comprising at least one antigen binding domain that binds to a tumor-associated antigen of any one of paras 33 to 36, wherein the agonistic ICOS-binding molecule comprises at least one antigen binding domain capable of specific binding to ICOS comprising a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25.

38. The agonistic ICOS-binding molecule comprising at least one antigen binding domain that binds to a tumor-associated antigen of any one of paras 33 to 37, wherein the agonistic ICOS-binding molecule comprising at least one antigen binding domain that binds to a tumor-associated antigen comprises at least one antigen binding domain comprising a heavy chain variable region (V_{H}ICOS) comprising an amino acid sequence of SEQ ID NO:26 and a light chain variable region (V_{L}ICOS) comprising an amino acid sequence of SEQ ID NO:27.

39. The agonistic ICOS-binding molecule comprising at least one antigen binding domain that binds to a tumor-associated antigen of any one of paras 33 to 38, wherein the agonistic ICOS-binding molecule comprising at least one antigen binding domain that binds to a tumor-associated antigen comprises an IgG Fc domain, specifically an IgG1 Fc domain or an IgG4 Fc domain.

40. The agonistic ICOS-binding molecule comprising at least one antigen binding domain that binds to a tumor-associated antigen of any one of paras 33 to 39, wherein the agonistic ICOS-binding molecule comprising at least one antigen binding domain that binds to a tumor-associated antigen comprises a Fc domain that comprises one or more amino acid substitution that reduces binding to an Fc receptor and/or effector function.

41. The agonistic ICOS-binding molecule comprising at least one antigen binding domain that binds to a tumor-associated antigen of any one of paras 33 to 40, wherein the agonistic ICOS-binding molecule comprises a first heavy chain comprising an amino acid sequence of SEQ ID NO:28, a first light chain comprising an amino acid sequence of SEQ ID NO:29, a second heavy chain comprising an amino acid sequence of SEQ ID NO:30, and a second light chain comprising an amino acid sequence of SEQ ID NO:31.

42. The agonistic ICOS-binding molecule comprising at least one antigen binding domain that binds to a tumor-associated antigen of any one of paras 33 to 40, wherein the agonistic ICOS-binding molecule comprises monovalent binding to a tumor associated target and bivalent binding to ICOS.

43. The agonistic ICOS-binding molecule comprising at least one antigen binding domain that binds to a tumor-associated antigen of any one of paras 33 to 40 or 42, wherein the agonistic ICOS-binding molecule comprises a first heavy chain comprising an amino acid sequence of SEQ ID NO:32, a second heavy chain comprising an amino acid sequence of SEQ ID NO:33, and a two light chains comprising an amino acid sequence of SEQ ID NO:29.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Generation of Bispecific Antibodies with a Monovalent or Bivalent Binding to ICOS and a Monovalent Binding to FAP

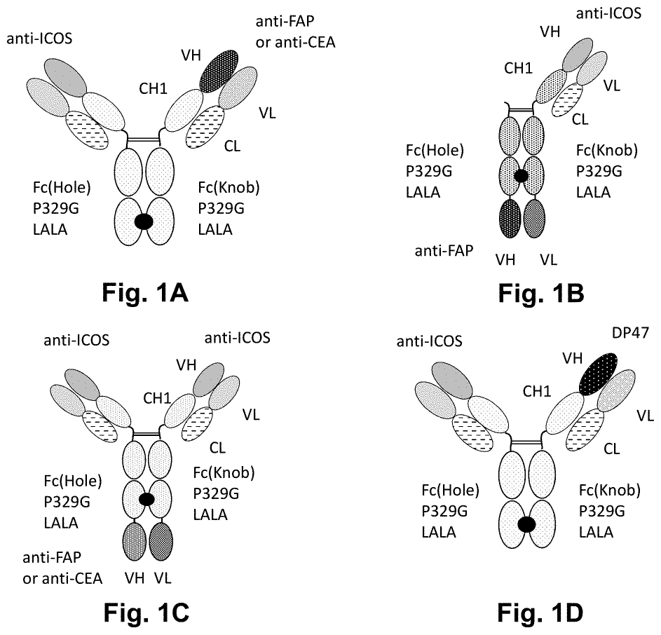

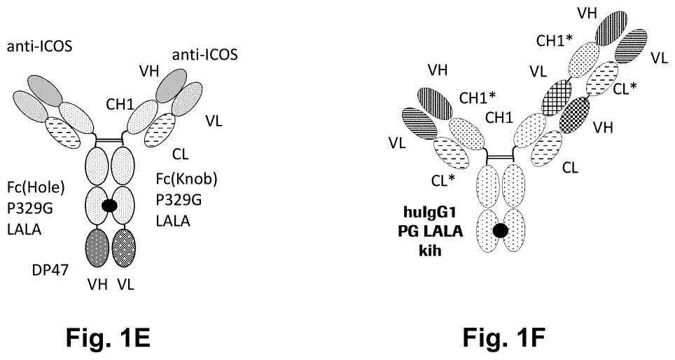

The following bispecific agonistic ICOS antibodies with monovalent or bivalent binding for ICOS and monovalent binding for FAP have been prepared as depicted in FIGS. 1A-1C, respectively.

A) FAP-ICOS_1+1, huIgG1 P329G LALA, monovalent ICOS (JMAb136), monovalent FAP (4B9) (FIG. 1A, SEQ ID Nos: 28-31)

B) FAP-ICOS_1+1_HT, huIgG1 P329G LALA, monovalent ICOS (JMAb136), monovalent FAP (4B9) c-terminal fused of the heavy chain C region (FIG. 1B, SEQ ID Nos: 29, 32, 66)

C) FAP-ICOS_2+1, huIgG1 P329G LALA, bivalent ICOS (JMAb136), monovalent FAP (4B9) (FIG. 1C, SEQ ID Nos: 29, 32, 33)

In example 1A, the HC1 of the FAP-ICOS_1+1 construct was comprised of the following components: VHCH1 of an anti-ICOS (JMAb136) followed by Fc hole. HC2 was comprised of VHCH1 of anti-FAP (4B9) followed by Fc knob.

In example 1B, the HC1 of the FAP-ICOS_1+1_HT construct was comprised of the following components: Fc hole, at which C-terminus a VH of anti-FAP binder (4B9) was fused. HC2 was comprised of VHCH1 of anti-ICOS (JMAb136) followed by Fc knob, at which C-terminus a VL of anti-FAP binder (4B9) was fused.

In example 1C, the HC1 of the FAP-ICOS_2+1 construct (example 1C) was comprised of the following components: VHCH1 of an anti-ICOS (JMAb136) followed by Fc hole, at which C-terminus a VH of anti-FAP binder (4B9) was fused. HC2 was comprised of VHCH1 of anti-ICOS (JMAb136) followed by Fc knob, at which C-terminus a VL of anti-FAP binder (4B9) was fused.

For the ICOS binder, the VH and VL sequences of clone JMAb136 were identical to those described in US patent publication No. 2008/0199466 A1.

Combination of the Fc knob with the Fc hole chain allows generation of a heterodimer. The Pro329Gly, Leu234Ala and Leu235Ala mutations have been introduced in the constant region of the knob and hole heavy chains to abrogate binding to Fcγ receptor according to the method described in International Patent Appl. Publ. No. WO2012/130831A1.

The amino acid sequences for bispecific agonistic ICOS antibodies can be found respectively in Tables 1, 2 and 3.

The targeted bispecific agonistic ICOS molecule encoding sequences were cloned into a plasmid vector driving expression of the insert from a Cytomegalovirus (CMV) promoter and containing a synthetic polyA sequence located at the 3′ end of the CDS. In addition, the vector contained an Epstein-Barr virus (EBV) oriP sequence for episomal maintenance of the plasmid.

The bispecific agonistic ICOS antibodies were produced by co-transfecting HEK293-EBNA cells with the mammalian expression vectors using polyethylenimine (PEI). The cells were transfected with the corresponding expression vectors at a 1:1:1:1 ratio (A: “VHCH1(JMAb136)-Fc knob chain”:“VLCL(JMAb136) Light chain”:“VHCH1 (4B9)-Fc hole chain”:“VLCL(4B9) Light chain”) or in a 1:1:1 ratio (B: “VHCH1 (JMAb136)-Fc knob chain-VH (4B9)”:“Fc hole chain-VL (4B9)”:“VLCL(JMAb136) Light chain”) or in a 1:1:2 ratio (C: “VHCH1 (JMAb136)-Fc knob chain-VH (4B9)”:“VHCH1 (JMAb136)-Fc hole chain-VL (4B9)”:“VLCL(JMAb136) Light chain”).

For production in 500 mL shake flasks, 400 million HEK293-EBNA cells were seeded 24 hours before transfection. For transfection cells were centrifuged for 5 minutes at 210×g, and the supernatant was replaced by pre-warmed CD CHO medium. Expression vectors were mixed in 20 mL CD CHO medium to a final amount of 200 μg DNA. After addition of 540 μL PEI, the solution was vortexed for 15 seconds and incubated for 10 minutes at room temperature.

Afterwards, cells were mixed with the DNA/PEI solution, transferred to a 500 mL shake flask and incubated for 3 hours at 37° C. in an incubator with a 5% CO₂ atmosphere. After the incubation, 160 mL of EX-CELL 293 (Sigma) medium was added and cells were cultured for 24 hours. One day after transfection 1 mM valproic acid and 7% Feed with supplements were added. After culturing for 7 days, the supernatant was collected by centrifugation for 15 minutes at 210×g. The solution was sterile filtered (0.22 m filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a Protein A MabSelectSure column (CV=5 mL, GE Healthcare) equilibrated with 40 mL 20 mM sodium phosphate, 20 mM sodium citrate pH 7.5. Unbound protein was removed by washing with at least 10 column volumes of 20 mM sodium phosphate, 20 mM sodium citrate containing buffer (pH 7.5). The bound protein was eluted using a linear pH-gradient of sodium chloride (from 20 to 100 mM) created over 15 column volumes of 20 mM sodium citrate, 100 mM NaCl, 100 mM Glycine, 0.01% Tween20 pH 3.0. The column was then washed with 10 column volumes of 20 mM sodium citrate, 100 mM NaCl, 100 mM Glycine, 0.01% Tween20 pH 3.0. The pH of collected fractions was adjusted by adding 1/40 (v/v) of 2 M Tris, pH 8.0. The protein was concentrated and filtered prior to loading on a HiLoad Superdex 50/600 S200 column (GE Healthcare) equilibrated with 20 mM Histidine, 140 mM NaCl, 0.01% Tween20 pH6.0.

The protein concentration of purified bispecific constructs was determined by measuring the OD at 280 nm, using the molar extinction coefficient calculated on the basis of the amino acid sequence. Purity and molecular weight of the bispecific constructs were analyzed by CE-SDS in the presence and absence of a reducing agent (Invitrogen, USA) using a LabChipGXII (Caliper). The aggregate content of bispecific constructs was analyzed using a TSKgel G3000 SW XL analytical size-exclusion column (Tosoh) equilibrated in a 25 mM K₂HPO₄, 125 mM NaCl, 200 mM L-Arginine Monohydrocloride, 0.02% (w/v) NaN₃, pH 6.7 running buffer at 25° C.

TABLE 1
amino acid sequences of bispecific 1 + 1 FAP(4B9)-targeted
anti-ICOS(JMAb136) human IgG1 P329G LALA (FIG 1A).

| SEQ ID | | |
|---|---|---|
| NO: | Description | Sequence |
| 28 | VHCH1(JMAbl | QVQLVQSGAEVKKPGASVKVSCKASGYTFTGYYMHWVRQA |
| | 36)- Fc knob | PGQGLEWMGWINPHSGGTNYAQKFQGRVTMTRDTSISTAY |
| | chain | MELSRLRSDDTAVYYCARTYYYDSSGYYHDAFDIWGQGTM |
| | | VTVSSASTKGPSVFPLAPSSKSTSGGTAALGCLVEDYFPEPVT |
| | | VSWNSGALTSGVHTFPAVLQSSGLYSLSSVVTVPSSSLGTQT |
| | | YICNVNHKPSNTKVDEKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSP |
| 29 | VLCL(JMAbl3 | DIQMTQSPSSVSASVGDRVTITCRASQGISRLLAWYQQKPGK |
| | 6) Light chain | APKLLIYVASSLQSGVPSRFSGSGSGTDFTLTISSLQPEDFATY |
| | | YCQQANSFPWTFGQGTKVEIKRTVAAPSVFIFPPSDRKLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 30 | VHCH1 (4B9)- | EIVLTQSPGTLSLSPGERATLSCRASQSVTSSYLAWYQQKPGQ |
| | Fc hole chain | APRLLINVGSRRATGIPDRFSGSGSGTDFTLTISRLEPEDFAVY |
| | | YCQQGIMLPPTFGQGTKVEIKSSASTKGPSVFPLAPSSKSTSG |
| | | GTAALGCLVKDYFPEPVTVSWNSGALTSGVHTFPAVLQSSGL |
| | | YSLSSVVTVPSSSLGTQTYICNVNHKPSNTKVDKKVEPKSCD |
| | | KTHTCPPCPAPEAAGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALGAPIEKTISKAKGQPRE |
| | | PQVCTLPPSRDELTKNQVSLSCAVKGFYPSDIAVEWESNGQP |
| | | ENNYKTTPPVLDSDGSFFLVSKLTVDKSRWQQGNVFSCSVM |
| | | HEALHNHYTQKSLSLSP |
| 31 | VLCL(4B9) | EVQLLESGGGLVQPGGSLRLSCAASGFTFSSYAMSWVRQAP |
| | Light chain | GKGLEWVSAIIGSGASTYYADSVKGRFTISRDNSKNTLYLQM |
| | | NSLRAEDTAVYYCAKGWFGGFNYWGQGTLVTVSSASVAAP |
| | | SVFIFPPSDEQLKSGTASVVCLLNNFYPREAKVQWKVDNALQ |
| | | SGNSQESVTEQDSKDSTYSLSSTLTLSKADYEKHKVYACEVT |
| | | HQGLSSPVTKSFNRGEC |

TABLE 2
amino acid sequences of bispecific 1 + 1 head-to-tail FAP(4B9)-
targeted anti-ICOS (JMAb136) human IgG1 P329G LALA (FIG. 1B).

| SEQ ID | | |
|---|---|---|
| NO: | Description | Sequence |
| 29 | VLCL(JMAb13 | DIQMTQSPSSVSASVGDRVTITCRASQGISRLLAWYQQKPGK |
| | 6) Light chain | APKLLIYVASSLQSGVPSRFSGSGSGTDFTLTISSLQPEDFATY |
| | | YCQQANSFPWTFGQGTKVEIKRTVAAPSVFIFPPSDRKLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 32 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | knob chain-VH | MELSRLRSDDTAVYYCARTYYYDSSGYYHDAFDIWGQGTM |
| | (4B9) | VTVSSASTKGPSVFPLAPSSKSTSGGTAALGCLVKDYFPEPVT |
| | | VSWNSGALTSGVIITFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGSGGGGSEVQLLESGGGLVQPGGSLRLSC |
| | | AASGFTFSSYAMSWVRQAPGKGLEWVSAIIGSGASTYYADS |
| | | VKGRFTISRDNSKNTLYLQMNSLRAEDTAVYYCAKGWFGGF |
| | | NYWGQGTLVTVSS |
| 66 | Fc hole chain- | DKTHTCPPCPAPEAAGGPSVFLFPPKPKDTLMISRTPEVTCVV |
| | VL (4B9) | VDVSHEDPEVKFNWYVDGVEVHNAKTKPREEQYNSTYRVV |
| | | SVLTVLHQDWLNGKEYKCKVSNKALGAPIEKTISKAKGQPR |
| | | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNHYTQKSLSLSPGGGGGSGGGGSGGGGSGGGGSEI |
| | | VLTQSPGTLSLSPGERATLSCRASQSVTSSYLAWYQQKPGQA |
| | | PRLLINVGSRRATGIPDRFSGSGSGTDFTLTISRLEPEDFAVYY |
| | | CQQGIMLPPTFGQGTKVEIK |

TABLE 3
amino acid sequences of bispecific 2 + 1 FAP(4B9)-targeted
anti-ICOS(JMAb136) human IgG1 P329G LALA (FIG. 1C)

| SEQ ID | | |
|---|---|---|
| NO: | Description | Sequence |
| 29 | VLCL(JMAbl3 | DIQMTQSPSSVSASVGDRVTITCRASQGISRLLAWYQQKPGK |
| | 6) Light chain | APKLLIYVASSLQSGVPSRFSGSGSGTDFTLTISSLQPEDFATY |
| | | YCQQANSFPWTFGQGTKVEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 32 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | knob chain-VH | MELSRLRSDDTAVYYCARTYYYDSSGYYHDAFDIWGQGTM |
| | (4B9) | VTVSSASTKGPSVFPLAPSSKSTSGGTAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGSGGGGSEVQLLESGGGLVQPGGSLRLSC |
| | | AASGFTFSSYAMSWVRQAPGKGLEWVSAIIGSGASTYYADS |
| | | VKGRFTISRDNSKNTLYLQMNSLRAEDTAVYYCAKGWFGGF |
| | | NYWGQGTLVTVSS |
| 33 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | hole chain-VL | MELSRLRSDDTAVYYCARTYYYDSSGYYHDAFDIWGQGTM |
| | (4B9) | VTVSSASTKGPSVFPLAPSSKSTSGGTAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVCTLPPSRDELTKNQVS |
| | | LSCAVKGFYPSDIAVEWESNGQPENNYKTTPPVLDSDGSFFL |
| | | VSKLTVDKSRWQQGNVFSCSVMHEALHNHYTQKSLSLSPGG |
| | | GGGSGGGGSGGGGSGGGGSEIVLTQSPGTLSLSPGERATLSC |
| | | RASQSVTSSYLAWYQQKPGQAPRLLINVGSRRATGIPDRFSG |
| | | SGSGTDFTLTISRLEPEDFAVYYCQQGIMLPPTFGQGTKVEIK |

### Example 2

### Generation of Bispecific Antibodies with a Monovalent or Bivalent Binding to ICOS and an Untargeted Moiety (Control Molecules)

The following bispecific agonistic ICOS antibodies with monovalent or bivalent binding for ICOS and containing an untargeted moiety were prepared similarly to the targeted formats, and as depicted in FIGS. 1D and 1E, respectively.

A) DP47-ICOS_1+1, huIgG1 P329G LALA, monovalent ICOS (JMAb136), monovalent DP47 (FIG. 1D, SEQ ID NO 28, 29, 67 and 68)

B) DP47-ICOS_2+1, huIgG1 P329G LALA, bivalent ICOS (JMAb136), monovalent DP47 (FIG. 1E, SEQ ID NO 29, 69 and 70)

In this example, the HC1 of the DP47-ICOS_1+1 construct was comprised of the following components, VHCH1 of JMAb136 as anti-ICOS antibody followed by Fc hole. HC2 was comprised of VHCH1 of DP47 as non-binding antibody followed by Fc knob.

The HC1 of the DP47-ICOS_2+1 construct was comprised of the following components: VHCH1 of an anti-ICOS (JMAb136) followed by Fc hole, at which C-terminus a VH of of a non-binding clone (DP47) was fused. HC2 was comprised of VHCH1 of anti-ICOS (JMAb136) followed by Fc knob, at which C-terminus a VL of a non-binding clone (DP47) was fused.

The untargeted bispecific agonistic ICOS molecules were prepared as described in Example 1 for the FAP(4B9)-targeted bispecific agonistic ICOS antibodies.

TABLE 4
amino acid sequences of bispecific 1 + 1 untargeted DP47
anti-ICOS(JMAb136) human IgG1 P329G LALA.

| SEQ ID | | |
|---|---|---|
| NO: | Description | Sequence |
| 28 | VHCH1(JMAb1 | QVQLVQSGAEVKKPGASVKVSCKASGYTFTGYYMHWVRQA |
| | 36)- Fc knob | PGQGLEWMGWINPHSGGTNYAQKFQGRVTMTRDTSISTAY |
| | chain | MELSRLRSDDTAVYYCARTYYYDSSGYYHDAFDIWGQGTM |
| | | VTVSSASTKGPSVFPLAPSSKSTSGGTAALGCLVEDYFPEPVT |
| | | VSWNSGALTSGVHTFPAVLQSSGLYSLSSVVTVPSSSLGTQT |
| | | YICNVNHKPSNTKVDEKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSP |
| 29 | VLCL(JMAb13 | DIQMTQSPSSVSASVGDRVTITCRASQGISRLLAWYQQKPGK |
| | 6) Light chain | APKLLIYVASSLQSGVPSRFSGSGSGTDFTLTISSLQPEDFATY |
| | | YCQQANSFPWTFGQGTKVEIKRTVAAPSVFIFPPSDRKLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 67 | VHCH1 (DP47)- | EIVLTQSPGTLSLSPGERATLSCRASQSVSSSYLAWYQQKPGQ |
| | Fc hole chain | APRLLIYGASSRATGIPDRFSGSGSGTDFTLTISRLEPEDFAVY |
| | | YCQQYGSSPLTFGQGTKVEIKSSASTKGPSVFPLAPSSKSTSG |
| | | GTAALGCLVKDYFPEPVTVSWNSGALTSGVHTFPAVLQSSGL |
| | | YSLSSVVTVPSSSLGTQTYICNVNHKPSNTKVDKKVEPKSCD |
| | | KTHTCPPCPAPEAAGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALGAPIEKTISKAKGQPRE |
| | | PQVCTLPPSRDELTKNQVSLSCAVKGFYPSDIAVEWESNGQP |
| | | ENNYKTTPPVLDSDGSFFLVSKLTVDKSRWQQGNVFSCSVM |
| | | HEALHNHYTQKSLSLSP |
| 68 | VLCL(DP47) | EVQLLESGGGLVQPGGSLRLSCAASGFTFSSYAMSWVRQAP |
| | Light chain | GKGLEWVSAISGSGGSTYYADSVKGRFTISRDNSKNTLYLQM |
| | | NSLRAEDTAVYYCAKGSGFDYWGQGTLVTVSSASVAAPSVF |
| | | IFPPSDEQLKSGTASVVCLLNNFYPREAKVQWKVDNALQSGN |
| | | SQESVTEQDSKDSTYSLSSTLTLSKADYEKHKVYACEVTHQG |
| | | LSSPVTKSFNRGEC |

TABLE 5
amino acid sequences of bispecific 2 + 1 untargeted DP47
anti-ICOS(JMAb136) human IgG1 P329G LALA (FIG. 1E).

| SEQ ID | | |
|---|---|---|
| NO: | Description | Sequence |
| 29 | VLCL(JMAb13 | DIQMTQSPSSVSASVGDRVTITCRASQGISRLLAWYQQKPGK |
| | 6) Light chain | APKLLIYVASSLQSGVPSRFSGSGSGTDFTLTISSLQPEDFATY |
| | | YCQQANSFPWTFGQGTKVEIKRTVAAPSVFIFPPSDRKLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 69 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | knob chain-VH | MELSRLRSDDTAVYYCARTYYYDSSGYYHDAFDIWGQGTM |
| | (DP47) | VTVSSASTKGPSVFPLAPSSKSTSGGTAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGSGGGGSEIVLTQSPGTLSLSPGERATLSC |
| | | RASQSVSSSYLAWYQQKPGQAPRLLIYGASSRATGIPDRFSGS |
| | | GSGTDFTLTISRLEPEDFAVYYCQQYGSSPLTFGQGTKVEIK |
| 70 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | hole chain-VL | MELSRLRSDDTAVYYCARTYYYDSSGYYHDAFDIWGQGTM |
| | (DP47) | VTVSSASTKGPSVFPLAPSSKSTSGG TAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVCTLPPSRDELTKNQVS |
| | | LSCAVKGFYPSDIAVEWESNGQPENNYKTTPPVLDSDGSFFL |
| | | VSKLTVDKSRWQQGNVFSCSVMHEALHNHYTQKSLSLSPGG |
| | | GGGSGGGGSGGGGSGGGGSEVQLLESGGGLVQPGGSLRLSC |
| | | AASGFTFSSYAMSWVRQAPGKGLEWVSAISGSGGSTYYADS |
| | | VKGRFTISRDNSKNTLYLQMNSLRAEDTAVYYCAKGSGFDY |
| | | WGQGTLVTVSS |

The results of the biochemical analysis of the bispecific molecules with a monovalent or bivalent binding to ICOS (JMAb136) and a monovalent binding to FAP (4B9) or DP47 produced as described in Examples 1 and 2 is summarized in Table 6.

TABLE 6
Biochemical analysis of bispecific
FAP-ICOS or DP47-ICOS molecules

| | Monomer | Yield | CE-SDS (non- |
|---|---|---|---|
| Molecule | [%] | [mg/L] | reduced) [%] |
| 1A) FAP-ICOS_1 + 1 | 91.0 | 13.9 | 100.0 |
| 1B) FAP-ICOS_1 + 1_HT | 97.3 | 2.6 | 98.3 |
| 1C) FAP-ICOS_2 + 1 | 96.6 | 6.3 | 94.0 |
| 1D) DP47-ICOS_1 + 1 | 91.0 | 37.5 | 98.4 |
| 1E) DP47-ICOS_2 + 1 | 100.0 | 11.7 | 98.5 |

### Example 3

### Generation of FAP-Targeted or Untargeted (Control) Bivalent Murine ICOS Ligand Constructs

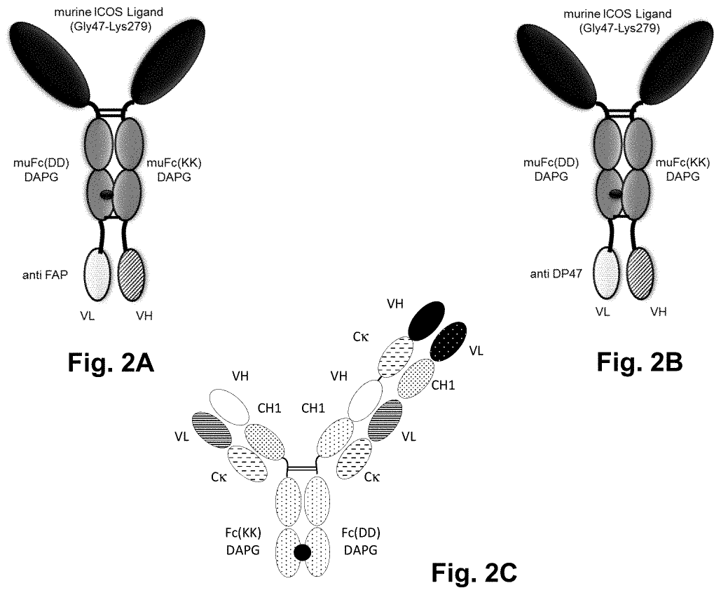

The following bispecific murine ICOS ligand (ICOSL) constructs containing a monovalent binding for FAP or an untargeted moiety have been prepared as depicted in FIGS. 2A and 2B.

2A) FAP-targeted mICOSL, mIgG1 DAPG, bivalent murine ICOSL (Gly47-Lys279) monovalent FAP (28H1) c-terminal fused of the heavy chain C region (FIG. 2A, SEQ ID NO 18-19)

2B) Untargeted mICOSL, mIgG1 DAPG, bivalent murine ICOSL (Gly47-Lys279), monovalent untargeted moiety c-terminal fused of the heavy chain C region (FIG. 2B, SEQ ID NO 20-21)

In this example the FAP-targeted mICOSL construct HC1 was comprised of murine ICOSL, murine Fc DAPG KK, at which C-terminus a VH of anti-FAP binder (28H1) was fused. HC2 was comprised of murine ICOSL followed by murine Fc DAPG DD, at which C-terminus a VL of anti-FAP binder (28H1) was fused.

The ICOSL amino acid sequence was obtained from Uniprot Q9JHJ8, from which Gly47 till Ser279 was used for cloning. The amino acid sequences for murine ICOS ligand (Gly47-Ser279) can be found respectively in Table 7.

Combination of the Fc DD with the Fc KK chain allows generation of a heterodimer. The DAPG mutations have been introduced in the constant region of the knob and hole heavy chains to abrogate binding to Fcγ receptor according to the method described in International Patent Appl. Publ. No. WO2012/130831A1.

The corresponding cDNAs were cloned into evitria's vector system using conventional (non-PCR based) cloning techniques. The evitria vector plasmids were gene synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with up to two sequencing reactions per plasmid depending on the size of the cDNA.)

Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter).

Secreted proteins were purified from cell culture supernatants by affinity chromatography using Protein A, followed by size exclusion chromatography. For affinity chromatography, the supernatant was loaded on a Protein A MabSelectSure column (CV=5 mL, GE Healthcare) equilibrated with 40 mL 20 mM sodium phosphate, 20 mM sodium citrate pH 7.5. Unbound protein was removed by washing with at least 10 column volumes of 20 mM sodium phosphate, 20 mM sodium citrate containing buffer (pH 7.5). The bound protein was eluted using a linear pH-gradient of sodium chloride (from 20 to 100 mM) created over 15 column volumes of 20 mM sodium citrate, 100 mM NaCl, 100 mM Glycine, 0.01% Tween20 pH 3.0. The column was then washed with 10 column volumes of 20 mM sodium citrate, 100 mM NaCl, 100 mM Glycine, 0.01% Tween20 pH 3.0. The pH of collected fractions was adjusted by adding 1/40 (v/v) of 2M Tris, pH8.0. The protein was concentrated and filtered prior to loading on a HiLoad Superdex 50/600 S200 column (GE Healthcare) equilibrated with 20 mM Histidine, 140 mM NaCl, 0.01% Tween20 pH6.0.

The protein concentration of purified bispecific constructs was determined by measuring the OD at 280 nm, using the molar extinction coefficient calculated on the basis of the amino acid sequence. Purity and molecular weight of the bispecific constructs were analyzed by CE-SDS in the presence and absence of a reducing agent (Invitrogen, USA) using a LabChipGXII (Caliper). The aggregate content of bispecific constructs was analyzed using a TSKgel G3000 SW XL analytical size-exclusion column (Tosoh) equilibrated in a 25 mM K₂HPO₄, 125 mM NaCl, 200 mM L-Arginine Monohydrochloride, 0.02% (w/v) NaN₃, pH 6.7 running buffer at 25° C.

TABLE 7
amino acid sequences of FAP(28H1)-targeted mICOSL, mIgG1 DAPG.

| SEQ ID | | |
|---|---|---|
| NO: | Description | Sequence |
| 71 | Murine ICOSL | ETEVGAMVGSNVVLSCIDPHRRHFNLSGLYVYWQIENPEV |
| | Linker muIgG1 | SVTYYLPYKSPGINVDSSYKNRGHLSLDSMKQGNFSLYLK |
| | Fc (DAPG KK) | NVTPQDTQEFTCRVFMNTATELVKILEEVVRLRVAANFSTP |
| | 4GS linker | VISTSDSSNPGQERTYTCMSKNGYPEPNLYWINTTDNSLIDT |
| | FAP(28H1) VH | ALQNNTVYLNKLGLYDVISTLRLPWTSRGDVLCCVENVAL |
| | | HQNITSISQAESFTGNNTKNPQETHNNELKGSPGSSSSSGSA |
| | | DGCKPCICTVPEVSSVFIFPPKPKDVLTITLTPKVTCVVVAIS |
| | | KDDPEVQFSWFVDDVEVHTAQTKPREEQINSTFRSVSELPI |
| | | MHQDWLNGKEFKCRVNSAAFGAPIEKTISKTKGRPKAPQV |
| | | YTIPPPKKQMAKDKVSLTCMITNFFPEDITVEWQWNGQPA |
| | | ENYKNTQPIMKTDGSYFVYSKLNVQKSNWEAGNTFTCSVL |
| | | HEGLHNHHTEKSLSHSPGGGGGSGGGGSGGGGSGGGGSEV |
| | | QLLESGGGLVQPGGSLRLSCAASGFTFSSHAMSWVRQAPG |
| | | KGLEWVSAIWASGEQYYADSVKGRFTISRDNSKNTLYLQM |
| | | NSLRAEDTAVYYCAKGWLGNFDYWGQGTLVTVSS |
| 72 | Murine ICOSL | ETEVGAMVGSNVVLSCIDPHRRHFNLSGLYVYWQIENPEV |
| | linker muIgG1 Fc | SVTYYLPYKSPGINVDSSYKNRGHLSLDSMKQGNFSLYLK |
| | (DAPG DD) 4GS | NVTPQDTQEFTCRVFMNTATELVKILEEVVRLRVAANFSTP |
| | linker | VISTSDSSNPGQERTYTCMSKNGYPEPNLYWINTTDNSLIDT |
| | FAP(28H1) VL | ALQNNTVYLNKLGLYDVISTLRLPWTSRGDVLCCVENVAL |
| | | HQNITSISQAESFTGNNTKNPQETHNNELKGSPGSSSSSGSA |
| | | GSPGSSSSSGSADGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTIS |
| | | KTKGRPKAPQVYTIPPPKEQMAKDKVSLTCMITNFFPEDIT |
| | | VEWQWNGQPAENYDNTQPIMDTDGSYFVYSDLNVQKSN |
| | | WEAGNTFTCSVLHEGLHNHHTEKSLSHSPGGGGGSGGGGS |
| | | GGGGSGGGGSEIVLTQSPGTLSLSPGERATLSCRASQSVSRS |
| | | YLAWYQQKPGQAPRLLIIGASTRATGIPDRFSGSGSGTDFTL |
| | | TISRLEPEDFAVYYCQQGQVIPPTFGQGTKVEIK |

TABLE 8
amino acid sequences of DP47-untargeted mICOSL, mIgG1 DAPG.

| SEQ ID | | |
|---|---|---|
| NO: | Description | Sequence |
| 73 | Murine ICOSL | ETEVGAMVGSNVVLSCIDPHRRHFNLSGLYVYWQIENPEVS |
| | Linker muIgG1 | VTYYLPYKSPGINVDSSYKNRGHLSLDSMKQGNFSLYLKN |
| | Fc (DAPG KK) | VTPQDTQEFTCRVFMNTATELVKILEEVVRLRVAANFSTPV |
| | G4S linker DP47 | ISTSDSSNPGQERTYTCMSKNGYPEPNLYWINTTDNSLIDTA |
| | VH | LQNNTVYLNKLGLYDVISTLRLPWTSRGDVLCCVENVALH |
| | | QNITSISQAESFTGNNTKNPQETHNNELKGSPGSSSSSGSAG |
| | | SPGSSSSSGSADGCKPCICTVPEVSSVFIFPPKPKDVLTITLTP |
| | | KVTCVVVAISKDDPEVQFSWFVDDVEVHTAQTKPREEQINS |
| | | TFRSVSELPIMHQDWLNGKEFKCRVNSAAFGAPIEKTISKTK |
| | | GRPKAPQVYTIPPPKKQMAKDKVSLTCMITNFFPEDITVEW |
| | | QWNGQPAENYKNTQPIMKTDGSYFVYSKLNVQKSNWEAG |
| | | NTFTCSVLHEGLHNHHTEKSLSHSPGGGGGSGGGGSGGGG |
| | | SGGGGSEIVLTQSPGTLSLSPGERATLSCRASQSVSSSYLAW |
| | | YQQKPGQAPRLLIYGASSRATGIPDRFSGSGSGTDFTLTISRL |
| | | EPEDFAVYYCQQYGSSPLTFGQGTKVEIK |
| 74 | Murine ICOSL | ETEVGAMVGSNVVLSCIDPHRRHFNLSGLYVYWQIENPEVS |
| | linker muIgG1 Pc | VTYYLPYKSPGINVD S SYKNRGHLSLDSMKQGNFSLYLKN |
| | (DAPG DD) 4GS | VTPQDTQEFTCRVFMNTATELVKILEEVVRLRVAANFSTPV |
| | linker DP47 VL | ISTSDSSNPGQERTYTCMSKNGYPEPNLYWINTTDNSLIDTA |
| | | LQNNTVYLNKLGLYDVISTLRLPWTSRGDVLCCVENVALH |
| | | QNITSISQAESFTGNNTKNPQETHNNELKGSPGSSSSSGSAG |
| | | SPGSSSSSGSADGCKPCICTVPEVSSVFIFPPKPKDVLTITLT |
| | | PKVTCVVVAISKDDPEVQFSWFVDDVEVHTAQTKPREEQIN |
| | | STFRSVSELPIMHQDWLNGKEFKCRVNSAAFGAPIEKTISKT |
| | | KGRPKAPQVYTIPPPKEQMAKDKVSLTCMITNFFPEDITVE |
| | | WQWNGQPAENYDNTQPIMDTDGSYFVYSDLNVQKSNWE |
| | | AGNTFTCSVLHEGLHNHHTEKSLSHSPGGGGGSGGGGSGG |
| | | GGSGGGGSEVQLLESGGGLVQPGGSLRLSCAASGFTFSSYA |
| | | MSWVRQAPGKGLEWVSAISGSGGSTYYADSVKGRFTISRD |
| | | NSKNTLYLQMNSLRAEDTAVYYCAKGSGFDYWGQGTLVTVSS |

The results of the biochemical analysis of the bispecific molecules comprising mlCOSL as produced as described in this example are summarized in Table 9.

TABLE 9
Biochemical analysis of bispecific FAP-
mICOS-L or DP47-mICOS-L molecules

| | | | | Analytical SEC |
|---|---|---|---|---|
| | Titer | Recovery | Yield | (HMW/Monomer/ |
| Molecule | [mg/l] | [%] | [mg/l] | LMW) |
| 2A | 28.01 | 48 | 2.69 | 2.9/97.1/0 |
| 2B | 22.30 | 82.96 | 3.72 | 4/96/0 |

### Example 4

### Preparation, Purification and Characterization of T-Cell Bispecific (TCB) Antibodies

### 4.1 Preparation of TCB Antibodies with Human or Humanized Binders

TCB molecules have been prepared according to the methods described in WO 2014/131712 A1 or WO 2016/079076 A1.

The preparation of the anti-CEA/anti-CD3 bispecific antibody (CEA CD3 TCB or CEA TCB) used in the experiments is described in Example 3 of WO 2014/131712 A1. CEA CD3 TCB is a “2+1 IgG CrossFab” antibody and is comprised of two different heavy chains and two different light chains. Point mutations in the CH3 domain (“knobs into holes”) were introduced to promote the assembly of the two different heavy chains. Exchange of the VH and VL domains in the CD3 binding Fab were made in order to promote the correct assembly of the two different light chains. 2+1 means that the molecule has two antigen binding domains specific for CEA and one antigen binding domain specific for CD3. CEACAM5 CD3 TCB has the same format, but comprises another CEA binder and comprises point mutations in the CH and CL domains of the CD3 binder in order to support correct pairing of the light chains.

CEA CD3 TCB comprises the amino acid sequences of SEQ ID NO:58, SEQ ID NO:59, SEQ ID NO:60 and SEQ ID NO:61. CEACAM5 CD TCB comprises the amino acid sequences of SEQ ID NO:62, SEQ ID NO:63, SEQ ID NO:64 and SEQ ID NO:65. A schematic scheme of the bispecific antibody in 2+1 format is shown in FIG. 1F.

### 4.2 Preparation of Anti-CEA/Anti-CD3 T Cell Bispecific Antibody in 2+1 Format (Bivalent for Murine CEA and Monovalent for Murine CD3)

The anti-CEA(CH1A1A 98/99 2F1)/anti-CD3(2C11) T cell bispecific 2+1 surrogate molecule was prepared consisting of one CD3-Fab, and two CEA-Fabs and a Fc domain, wherein the two CEA-Fabs are linked via their C-termini to the hinge region of said Fc part and wherein the CD3-Fab is linked with its C-terminus to the N-terminus of one CEA-Fab. The CD3 binding moiety is a crossover Fab molecule wherein either the variable or the constant regions of the Fab light chain and the Fab heavy chain are exchanged.

The Fc domain of the murine surrogate molecule is a mu IgG1 Fc domain, wherein DDKK mutations have been introduced to enhance antibody Fc heterodimer formation as inter alia described by Gunasekaran et al., J. Biol. Chem. 2010, 19637-19646. The Fc part of the first heavy chain comprises the mutations Lys392Asp and Lys409Asp (termed Fc-DD) and the Fc part of the second heavy chain comprises the mutations Glu356Lys and Asp399Lys (termed Fc-KK). The numbering is according to Kabat EU index. Furthermore, DAPG mutations were introduced in the constant regions of the heavy chains to abrogate binding to mouse Fc gamma receptors according to the method described e.g. in Baudino et al. J. Immunol. (2008), 181, 6664-6669, or in WO 2016/030350 A1. Briefly, the Asp265Ala and Pro329Gly mutations have been introduced in the constant region of the Fc-DD and Fc-KK heavy chains to abrogate binding to Fc gamma receptors (numbering according to Kabat EU index; i.e. D265A, P329G).

TABLE 10
amino acid sequences of murine anti-CEA/anti-CD3
T cell bispecific antibody.

| SEQ ID | | |
|---|---|---|
| NO: | Description | Sequence |
| 75 | VHCH1(CH1A1 | QVQLVQSGAEVKKPGASVKVSCKASGYTFTEFGMNWVRQ |
| | A 98/99 2F1)- | APGQGLEWMGWINTKTGEATYVEEFKGRVTFTTDTSTSTA |
| | Fc(KK) DAPG | YMELRSLRSDDTAVYYCARWDFAYYVEAMDYWGQGTTV |
| | chain | TVSSAKTTPPSVYPLAPGSAAQTNSMVTLGCLVKGYFPEPV |
| | | TVTWNSGSLSSGVHTFPAVLQSDLYTLSSSVTVPSSTWPSQ |
| | | TVTCNVAHPASSTKVDKKIVPRDCGCKPCICTVPEVSSVFIF |
| | | PPKPKDVLTITLTPKVTCVVVAISKDDPEVQFSWFVDDVEV |
| | | HTAQTKPREEQINSTFRSVSELPIMHQDWLNGKEFKCRVNS |
| | | AAFGAPIEKTISKTKGRPKAPQVYTIPPPKKQMAKDKVSLT |
| | | CMITNFFPEDITVEWQWNGQPAENYKNTQPIMKTDGSYFV |
| | | YSKLNVQKSNWEAGNTFTCSVLHEGLHNHHTEKSLSHSP |
| 76 | VLCL | DIQMTQSPSSLSASVGDRVTITCKASAAVGTYVAWYQQKP |
| | (CH1A1A 98/99 | GKAPKLLIYSASYRKRGVPSRFSGSGSGTDFTLTISSLQPEDF |
| | 2F1) Light chain | ATYYCHQYYTYPLFTFGQGTKLEIKRADAAPTVSIFPPSSEQ |
| | | LTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| 77 | VHCL VHCH1 | EVQLVESGGGLVQPGKSLKLSCEASGFTFSGYGMHWVRQA |
| | (2C11- | PGRGLESVAYITSSSINIKYADAVKGRFTVSRDNAKNLLFLQ |
| | CH1A1 A 98/99 | MNILKSEDTAMYYCARFDWDKNYWGQGTMVTVSSASDA |
| | 2F1)- | APTVSIFPPSSEQLTSGGASVVCFLNNFYPKDINVKWKIDGS |
| | Fc(DD) DAPG | ERQNGVLNSWTDQDSKDSTYSMSSTLTLTKDEYERHNSYT |
| | chain | CEATHKTSTSPIVKSFNRNECGGGGSGGGGSQVQLVQSGAE |
| | | VKKPGASVKVSCKASGYTFTEFGMNWVRQAPGQGLEWM |
| | | GWINTKTGEATYVEEFKGRVTFTTDTSTSTAYMELRSLRSD |
| | | DTAVYYCARWDFAYYVEAMDYWGQGTTVTVSSAKTTPPS |
| | | VYPLAPGSAAQTNSMVTLGCLVKGYFPEPVTVTWNSGSLS |
| | | SGVHTFPAVLQSDLYTLSSSVTVPSSTWPSQTVTCNVAHPA |
| | | SSTKVDKKIVPRDCGCKPCICTVPEVSSVFIFPPKPKDVLTIT |
| | | LTPKVTCVVVAISKDDPEVQFSWFVDDVEVHTAQTKPREE |
| | | QINSTFRSVSELPIMHQDWLNGKEFKCRVNSAAFGAPIEKTI |
| | | SKTKGRPKAPQVYTIPPPKEQMAKDKVSLTCMITNFFPEDIT |
| | | VEWQWNGQPAENYDNTQPIMDTDGSYFVYSDLNVQKSN |
| | | WEAGNTFTCSVLHEGLHNHHTEKSLSHSP |
| 78 | VLCH1 (2C11) | DIQMTQSPSSLPASLGDRVTINCQASQDISNYLNWYQQKPG |
| | Light chain | KAPKLLIYYTNKLADGVPSRFSGSGSGRDSSFTISSLESEDIG |
| | | SYYCQQYYNYPWTFGPGTKLEIKSSAKTTPPSVYPLAPGSA |
| | | AQTNSMVTLGCLVKGYFPEPVTVTWNSGSLSSGVHTFPAV |
| | | LQSDLYTLSSSVTVPSSTWPSQTVTCNVAHPASSTKVDKKI |
| | | VPRDC |

### Example 5

### 5.1 PBMC Isolation

PBMCs were isolated from fresh blood of healthy donors. Briefly, blood was diluted 2:1 with PBS. About 30 ml of the blood/PBS mixture was layered on 15 ml of Histopaque and centrifuged for 30 min at 450 g without brake. The lymphocytes were collected with a 10 ml pipette into 50 ml tubes containing PBS. The tubes were filled up to 50 ml with PBS and centrifuged 10 min at 350 g. The supernatant was discarded, the pellet re-suspended in 50 ml PBS and centrifuged for 10 min at 300 g. The washing step was repeated once. The cells were re-suspended in RPMI containing 10% FBS and 1% GlutaMax and stored at 37° C., 5% CO₂ in the incubator until assay start (not longer than 24 h).

### 5.2 Splenocyte Isolation

Spleens of C57Bl/6 mice or hCEA(HO)Tg mice were transferred into gentleMACS C-tubes (Miltenyi) and MACS buffer (PBS+0.5% BSA+2 mM EDTA) was added to each tube. Spleens were dissociated using the GentleMACS Dissociator, tubes were spun down shortly and cells were passed through a 100 m nylon cell strainer. Thereafter, tubes were rinsed with 3 ml RPMI1640 medium (SIGMA, Cat.-No. R7388) and centrifuged for 8 min at 350×g. The supernatant was discarded, the cell suspension passed through a 70 m nylon cell strainer and washed with medium. After another centrifugation (350×g, 8 min), supernatants were discarded and 5 ml ACK Lysis Buffer was added. After 5 min incubation at RT cells were washed with RPMI medium.

Afterwards the cells were re-suspended and the pellets pooled in assay medium (RPMI1640, 2% FBS, 1% Glutamax), for cell counting (Vi-Cell-Settings leukocytes, 1:10 dilution).

### Example 6

### ICOS Expression on Healthy Human T Cell Subpopulations or Tumour-Infiltrating Lymphocytes of Various Tumour Indications

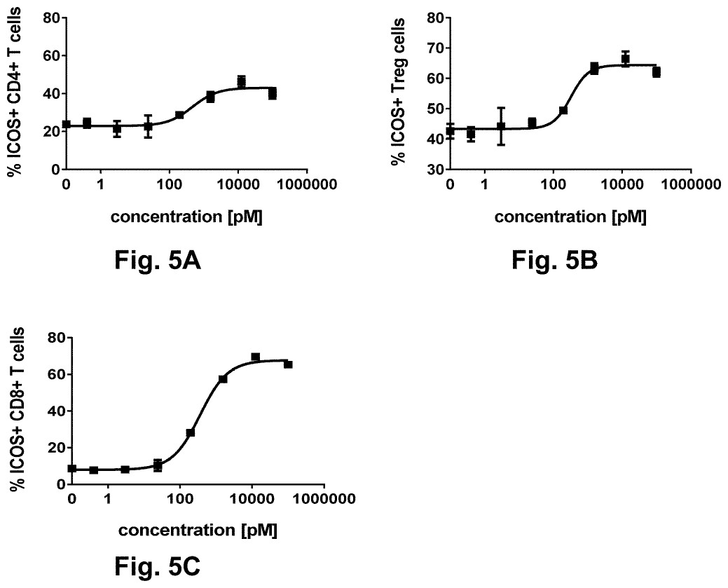

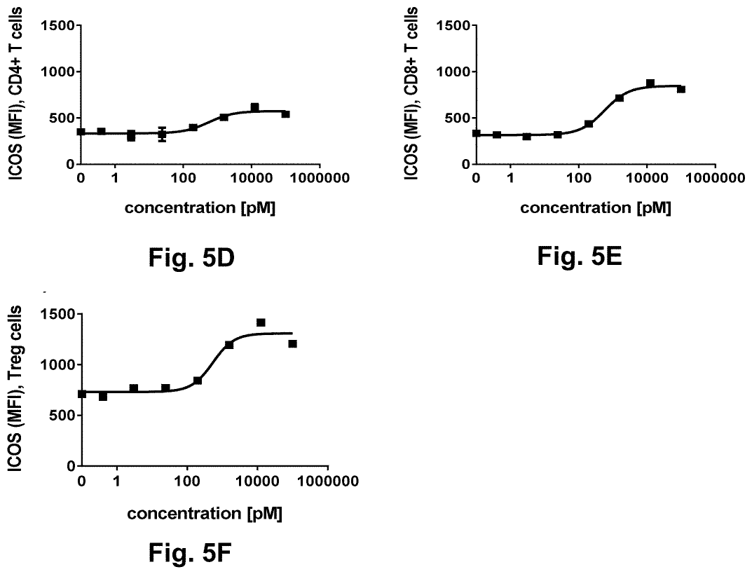

To compare the relative intensities of ICOS expressed on different T cell populations on healthy PBMCs versus TILs of different tumour indications, ICOS expression was evaluated using multi-color flow cytometry. In addition, for some patients PBMCs were isolated from normal tissue adjacent to the tumour tissue and ICOS expression was assessed as well (FIGS. 5A to 5D). Tumour samples were derived from the following indications: pancreatic cancer (PancCa), lymph node metastasis (portio), penis adenocarcinoma, renal cell carcinoma (RCC), colorectal cancer (CRC), melanoma, mesothelioma, sarcomatoid soft tissue neoplasia, paracardiac tumour node, colon metastasis of an ovarian cancer, seminoma, head & neck cancer, Thymom, gastrointestinal stroma tumor (GIST), lung metastasis of an adenoid cystic carcinoma, kidney cancer, a rectum metastasis of a liver adenocarcinoma, small intestine cancer, lipomatous soft tissue tumour, and lung cancer (LC).

6.1 Digestion of Human Tumor or Adjacent Normal Tissue Samples

For the analysis of ICOS expression on T-cells from tumour or normal tissue, samples were digested and single-cell suspensions were analyzed by flow cytometry.

Briefly, a digestion mix was prepared, using 4.4 ml tissue storage solution (#130-100-008, Miltenyi Biotech), 5 ml Accutase (# A6964, Sigma), 333 l 1% BSA (# A9576, Sigma) and 260 μl enzyme mix (275 U/ml Collagenase IV, # LS004189, Worthington, 10 U/ml DNAse I Type 4, #10 U/ml DNAse I Type 4, Sigma and 471 U/ml Hyaluronidase # H6254, Sigma).

Tumor, respective adjacent normal tissue pieces were cut in small pieces at room temperature and incubated in the digestion mix for 40 minutes at 37° C., using a rotating device. Cells were further separated, using a 70 m cell strainer (#352350, Corning). The cell strainer was washed with 10 ml cold PBS (#352350, GIBCO) and the cell suspension was centrifuged for 10 minutes at 250×g at 4° C. After an additional washing step with cold PBS, followed by centrifugation as described above, erythrocytes were lysed by incubating the cell pellets for 5 to 15 minutes in 1× PharmLyse buffer (#555899, BD Biosciences) at room temperature. The lysis was stopped by addition of cold PBS and centrifugation for 10 minutes at 250×g and 4° C. Cells were washed a second time with cold PBS and number of living cells was determined using trypan blue staining.

6.2 Digestion of Human Tumor or Adjacent Normal Tissue Samples

Expression level of ICOS on human T-cells from healthy donors, on TILs or on T-cells isolated from normal tissue of cancer patients was determined as follows: 0.05-0.3 million cells were washed once, using PBS and centrifugation for 5 minutes at 350×g. For the discrimination of live and dead cells, PBMCs were incubated with Zombie UV (#423108, BioLegend) for 20 minutes at room temperature. Cells were washed with PBS and FACS buffer (PBS, containing 2% FCS, 5 mM EDTA and 0.25% sodium acide) and stained for CD4 (#317438, BioLegend), CD8 (#301044, BioLegend) and ICOS (#313520, BioLegend) for 20 minutes at room temperature. After two washing steps with FACS buffer, staining was fixed by incubation of cells in 2% PFA-containing PBS for 15 minutes at room temperature. Cells were washed with FACS buffer once and analyzed by flow cytometry (FACS Fortessa instrument, equipped with Diva Software).

### Example 7

### CEACAM5-TCB Mediated Up-Regulation of ICOS on Peripheral T-Cells and TILs of Tumor Patients (FACS)

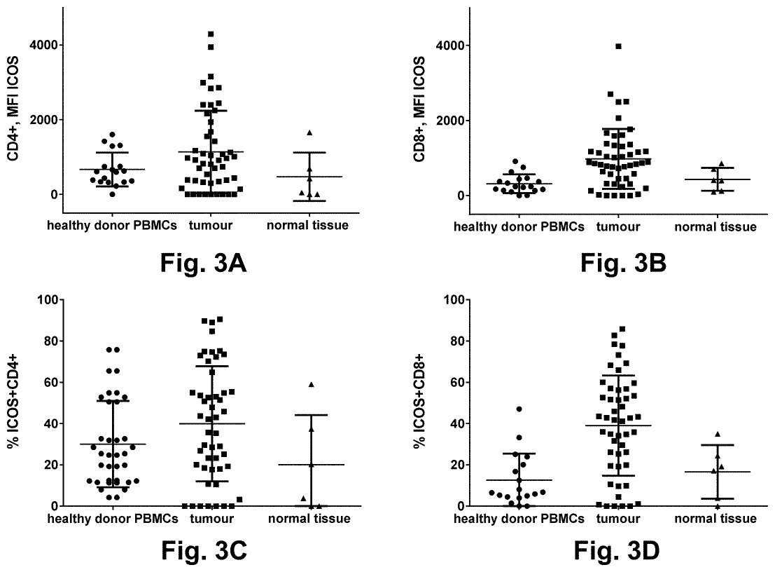

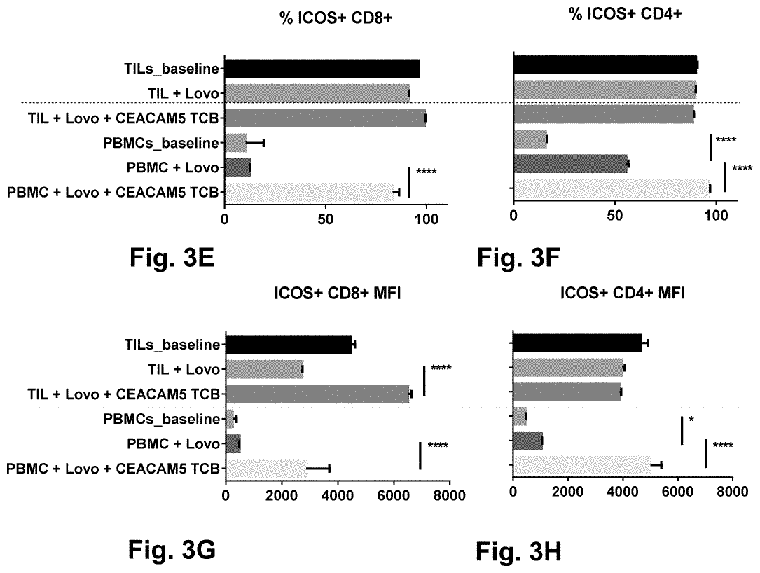

Enhanced expression of human ICOS upon TCB-mediated activation of human T-cells in vitro was evaluated by flow cytometry (FIGS. 3E-3H). Briefly, 0.2 Mio PBMCs or TILs isolated from an ovarian cancer patient with a colon metastasis (6.6.1 and 6.4) were re-suspended in DMEM, including 10% FCS, 1% Glutamax, 1 mM Sodium Pyruvate, 1×NEAA+Pen/Strep and incubated with 0.04 Mio CEA-expressing Lovo tumor cells (# CCL-229, ATCC) and 10 nM CEACAM5-TCB, using flat-bottom-96-well plates.

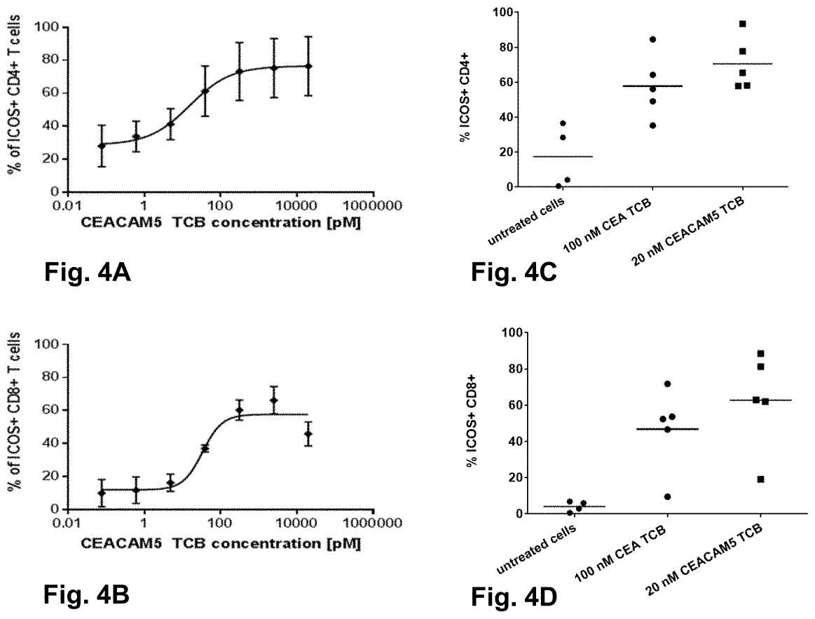

In another example (FIGS. 4A and 4B), 0.25 million PBMCs of healthy donors were incubated with 0.025 Mio CEA-positive MKN45 and varying concentrations of CEACAM5-TCB (0.1 pM to 20 nM) for 48 hours in flat-bottom-96-well plates. Up-regulation of ICOS was assessed after 48 h of incubation at 37° C., 5% CO₂ after staining of surface markers CD4, CD8 and ICOS, as described below.

PBMCs were harvested from assay plates and transferred to fresh round-bottom 96-well-plates for subsequent staining at 4° C. Cells were washed once with FACS buffer and then stained for 30 min at 4° C., using anti-human CD4 (#300506, BioLegend), anti-human CD8 (#344722, BioLegend), anti-human CD25 (#356120, BioLegend), anti-human CD69 (#310930, BioLegend), and anti-human ICOS (#310931 BioLegend) according to the manufacturers' instructions. Cells were washed twice using FACS buffer and fixed for 15 minutes at room temperature in FACS buffer, containing 2% PFA. After one washing step with FACS buffer, cells were re-suspended in FACS buffer and analyzed using a BD FACS Fortessa machine.

### Example 8

### Murine CEA-TCB Mediated Up-Regulation of Murine ICOS on Murine Splenocytes (FACS)

Enhanced expression of murine ICOS upon TCB-mediated activation of murine T-cells ex vivo was evaluated by flow cytometry (FIGS. 5A-5F). For this, a classical tumor cell lysis experiment was performed as described in Example 14. After 48 h, splenocytes were harvested and transferred into a fresh round-bottom 96-well plate for subsequent staining.

Cells were washed once with PBS and stained using the UV Zombie dye (#423108 BioLegend), diluted 1:1000 in PBS for 30 minutes at 4° C. Cells were washed with FACS buffer twice and surface staining was performed for 30 minutes at 4° C., using anti-mouse TCRD (#109228 BioLegend), anti-mouse ICOS (#135220 BioLegend), anti-mouse CD4 (#100422 BioLegend), anti-mouse CD8 (#100747 BioLegend) according to the manufacturers' instructions. Cells were washed with FACS buffer twice and intracellular staining was started with addition of Perm/Fix Buffer (#421403 BioLegend) for 30-45 minutes at room temperature protected from light. Cells were washed three times with Perm Buffer (#421402 BioLegend) and stained in perm buffer, containing anti-murine FoxP3 (#320014 BioLegend). Cells were washed twice using FACS buffer and fixed for 15 minutes at room temperature in FACS buffer, containing 2% PFA. After one washing step with FACS buffer, cells were re-suspended in FACS buffer and analyzed using a BD FACS Fortessa machine.

### Example 9

### Binding of FAP-Targeted ICOS and Murine ICOS-L, Respective Untargeted Reference Molecules to FAP- and ICOS-Expressing Cells

The binding of several FAP-ICOS molecules prepared in Examples 1 and 2 was tested using human fibroblast activating protein (huFAP) expressing NIH/3T3-huFAP clone 19. This cell line was generated by the transfection of the mouse embryonic fibroblast NIH/3T3 cell line (ATCC CRL-1658) with the expression vector pETR4921 to express huFAP under 1.5 g/mL Puromycin selection. The binding to human ICOS was tested with activated human T-Cells.

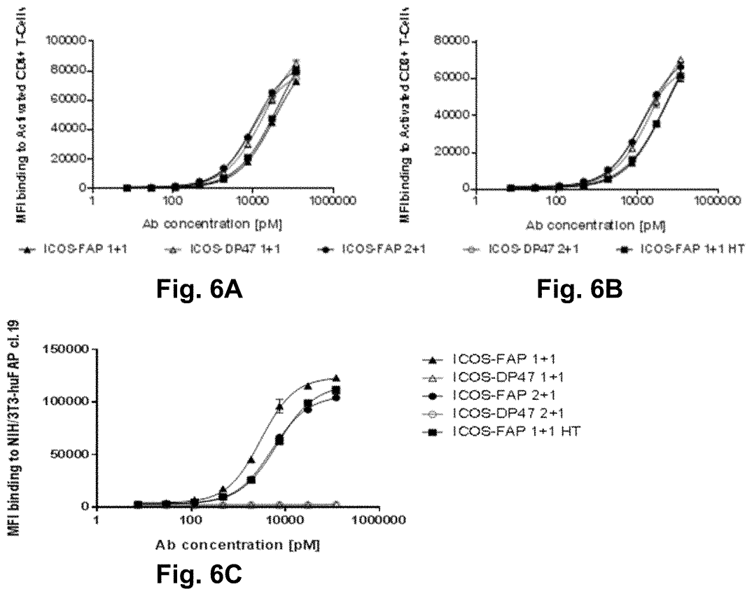

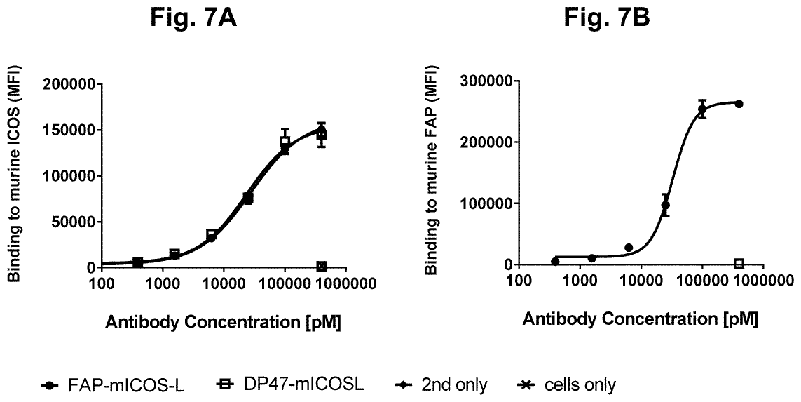

Furthermore, mICOS-L-targeting bispecific molecules prepared in Example 3 were tested for their binding to murine FAP (mFAP), using 3T3-mFAP cells (parental cell line ATCC # CCL-92, modified to stably overexpress murine FAP, and the binding to murine ICOS-expressing cells was tested using CHO mICOS (parental cell line CHO-kl ATCC # CCL-61, modified to stably overexpress murine ICOS). Briefly, cells were harvested, counted, checked for viability and re-suspended at 1 million cells per ml in FACS buffer (PBS with 0.1% BSA). 100 μl of the cell suspension (containing 0.1 million cells) were incubated in round-bottom 96-well plates for 30 min at 4° C. with increasing concentrations of the FAP-targeted ICOS or mICOS-L constructs (7 pM-120 nM for the binding of FAP-ICOS constructs to T-Cells/3T3-huFAP cells, respective 4 pM-300 nM for the binding of mICOS-L constructs), cells were washed twice with cold PBS 0.1% BSA, re-incubated for further 30 min at 4° C. with the PE-conjugated, donkey anti human H+L PE (Jackson Immuno Research Lab #709-116-149) and washed twice with cold PBS 0.1% BSA. The staining was fixed for 20 min at 4° C. in the dark, using 75 μl of 1% PFA in FACS buffer per well. Fluorescence was analyzed by FACS using a FACS Fortessa (Software FACS Diva). Binding curves were obtained using GraphPadPrism6 and are shown in FIGS. 6A-6C and 7A-7B, respectively.

The EC₅₀ values, as determined by binding of different FAP- or DP47-ICOS molecules to human ICOS- and human FAP-expressing cells are shown in Table 11 and the EC50 values determined for binding of FAP-mICOS-L to murine ICOS-expressing CHO transfectants and murine FAP-expressing 3T3 cells are presented in Table 12 below.

TABLE 11
EC₅₀ values of binding of different FAP- or DP47-ICOS
molecules to human ICOS-and human FAP-expressing cells

| | Human ICOS | Human ICOS | |
|---|---|---|---|
| | on CD4⁺ | on CD8+ | |
| | T-Cells | T-Cells | Human FAP |
| Molecule | EC₅₀ (nM) | EC₅₀ (nM) | EC₅₀ (nM) |
| FAP-ICOS_1 + 1 | 34.86 | 50.72 | 2.98 |
| FAP-ICOS_1 + 1_HT | 42.62 | 56.43 | 6.73 |
| FAP-ICOS_2 + 1 | 10.74 | 14.38 | 5.19 |
| DP47-ICOS_1 + 1 | 19.85 | 25.7 | no binding |
| DP47-ICOS_2 + 1 | 9.99 | 12.36 | no binding |

TABLE 12
EC₅₀ values, binding of FAP-mICOS-L to murine ICOS-expressing
CHO transfectants and murine FAP-expressing 3T3 cells

| Molecule | Target | EC₅₀ (nM) |
|---|---|---|
| FAP-mICOS-L | Murine ICOS | 28.05 |
| DP47-mICOS-L | Murine ICOS | 23.57 |
| FAP-mICOS-L | Murine FAP | 71.8 |

### Example 10

### In Vitro Functional Characterization of FAP-Targeted ICOS, Respective mICOS-L Molecules

Several cell-based in vitro assays were performed to evaluate the activity of FAP-targeted ICOS or mICOS-L molecules versus their corresponding untargeted (DP47) reference molecules.

The assays were designed to show additional agonistic/co-stimulatory activity of the anti-ICOS bispecific molecules in presence of T-cell bispecific-(TCB) mediated activation of T-cells.

1. Jurkat assay (reporter cell line with NFAT-regulated expression of luciferase, induced upon engagement of the CD3/TCR and ICOS), wherein ICOS IgG molecules, plate-bound vs. in solution and in absence versus presence of a coated CD3 IgG stimulus were measured

2. Primary human PBMC co-culture assay, wherein FAP-targeted ICOS molecules, cross-linked by simultaneous binding to human ICOS on T-cells and human FAP, expressed on 3T3-hFAP cells (parental cell line ATCC # CCL-92, modified to stably overexpress human FAP), in the presence of a TCB molecule being crosslinked by simultaneous binding to CD3 on T-cells and human CEA on tumor cells were tested. The FAP-targeted cross-linked ICOS molecules versus untargeted DP47-ICOS molecules were measured in solution, in absence versus presence of a TCB molecule and CEA-positive tumor cells. T-cell activation, T-cell differentiation or T-cell proliferation as determined by flow cytometry were obtained as readouts.

3. Primary murine splenocyte co-culture assay, wherein FAP-targeted mICOS-L molecule, cross-linked by simultaneous binding to murine ICOS on T-cells and murine FAP, expressed on 3T3-mFAP cells (parental cell line ATCC # CCL-92, modified to stably overexpress murine FAP), in presence of a TCB molecule being crosslinked by simultaneous binding to mCD3 on murine T-cells and human CEA on MC38-hCEA tumor cells was measured.

### Example 11

### Jurkat Reporter Cell Line Assay

The Dependency on a simultaneous TCR engagement was assessed by using an engineered Jurkat Cell Line expressing Luciferase in response to NFAT nuclear translocation.

GloResponse Jurkat NFAT-RE-luc2P (Promega # CS176501) reporter cell line was preactivated to induce ICOS expression using Cell Culture Flasks coated with 1.5 μg/ml aCD3 (BioLegend #317304) and 2 μg/ml aCD28 (BioLegend, #302914) in JurkatNFAT culture Medium (RPMI1640 medium containing 10% FCS, 1% GluMax, 25 mM HEPES, 1×NEAA, 1% So-Pyruvate; selection: 200 μg/ml Hygromycin B).

Cells were starved (JurkatNFAT culture Medium without Stimulation) overnight before the assay. Assay Plates (Greiner, 96 well, white wall, clear bottom; #655098) were coated (4° C. overnight) simultaneously with either 0.5 μg/ml aCD3 (BioLegend #317304) plus FAP-ICOS/DP47-ICOS molecules or FAP-ICOS/DP47-ICOS molecules only at the indicated concentrations (range of 4 pM-65000 pM in triplicates). The next day the plates were washed once with DPBS (Gibco, #14190136) and 0.1 Mio stimulated and starved GloResponse Jurkat NFAT-RE-luc2P were added. NFAT mediated signaling was assessed after 5 h of incubation at 37° C., 5% CO₂ by Luminescence Reading using Promega OneGlo Assay System (Promega, #E6120) according to manufacturer instructions. Plates were read on a Tecan Sparkl M Plate Reader (Luminescence Reading, 1000 ms Integration Time, Auto Attenuation Setting).

### Example 12

### Primary Human PBMC Co-Culture Assay

Enhanced T-cell activation, T-Cell proliferation or T-Cell differentiation mediated by combining CEACAM5-TCB and the FAP-ICOS molecules was assessed on CEA-expressing MKN45 (DSMZ # ACC 409) cells and huFAP-expressing Fibroblasts (NIH/3T3-huFAP clone 19).

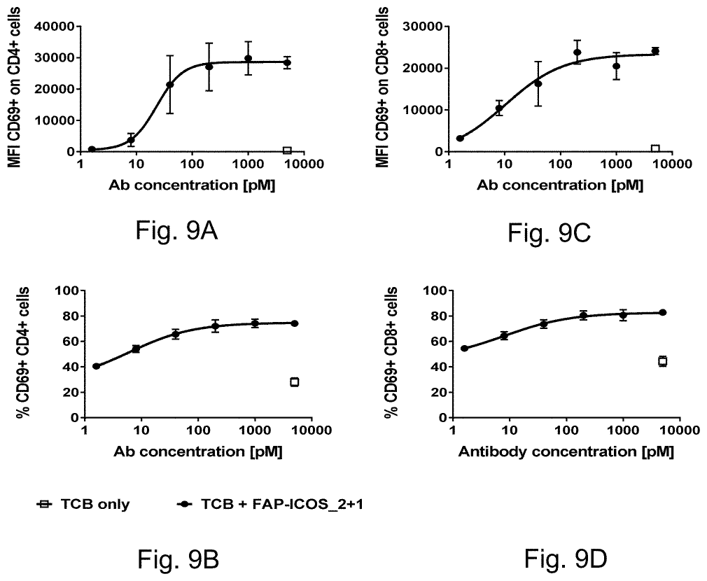

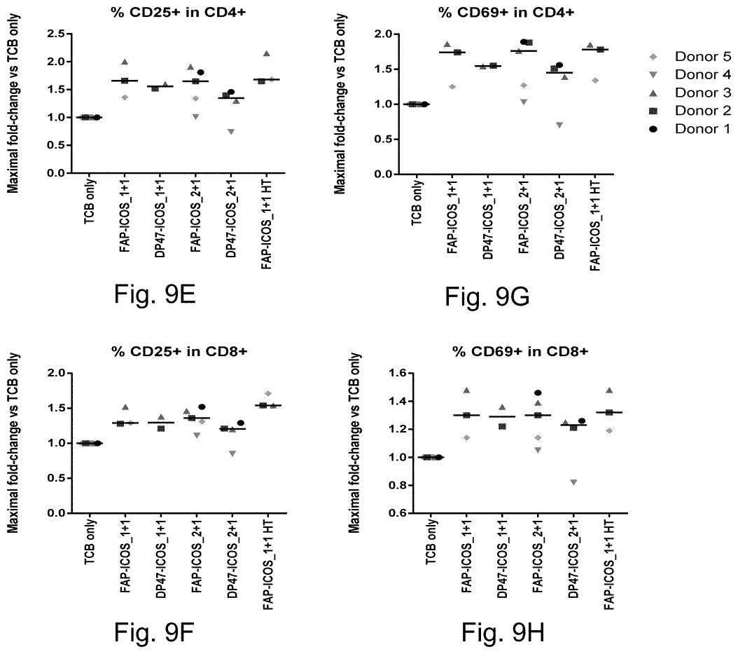

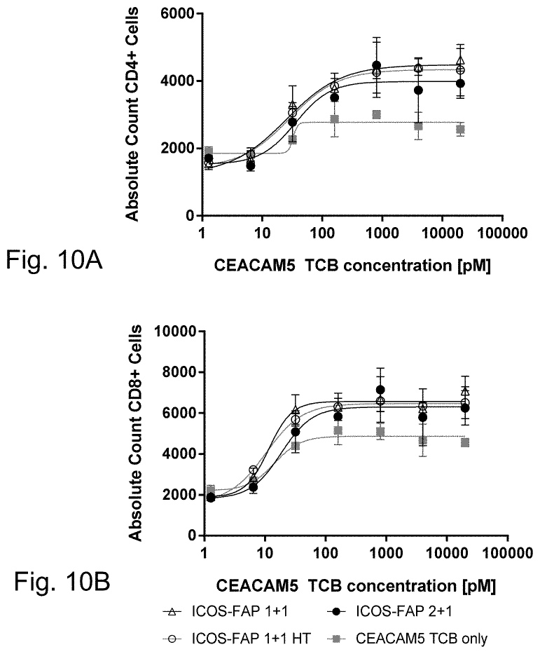

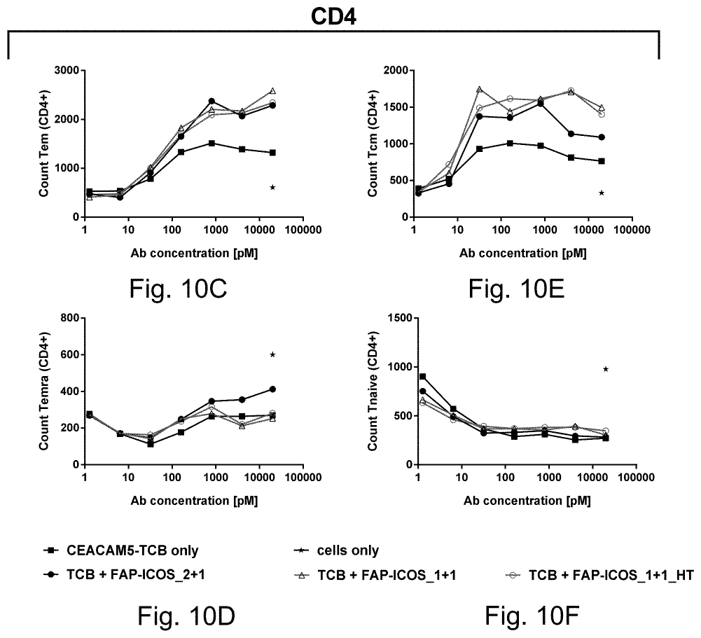

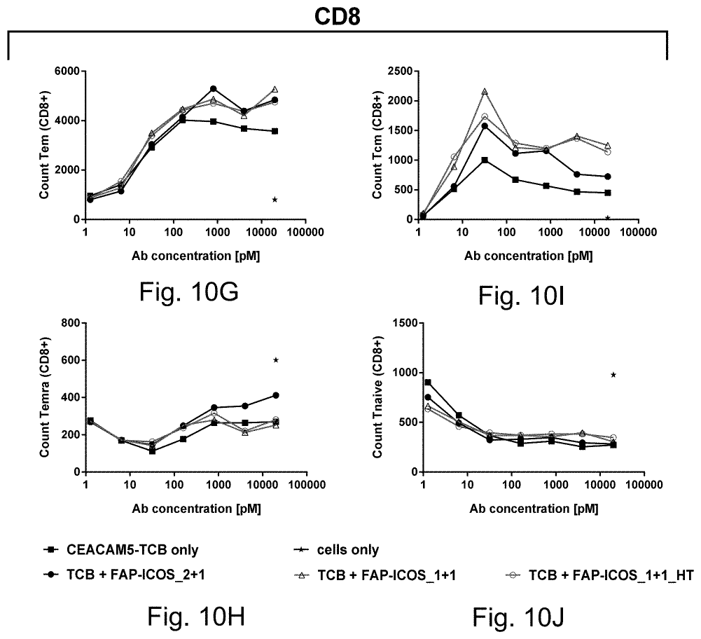

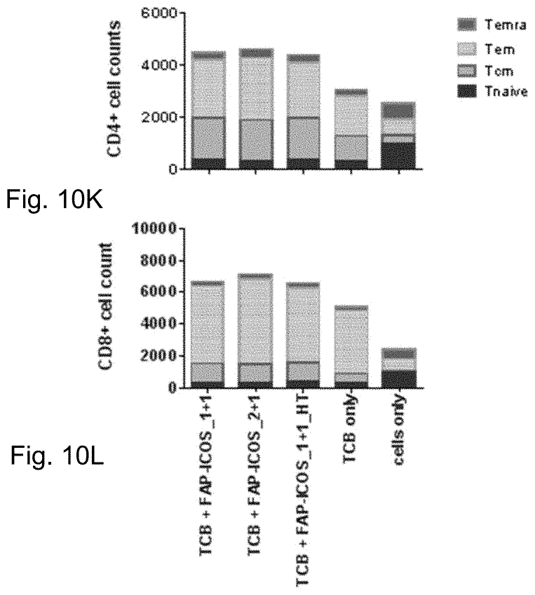

Human PBMCs were used as effector cells. T-Cell Activation was detected after 48 h, T-Cell Proliferation and -Differentiation after 96 h of incubation with FAP-ICOS and CEACAM5-TCB. Briefly, adherent target cells and Fibroblasts were harvested with Trypsin/EDTA, washed, and plated at density of 10 000 cells/well using flat-bottom 96-well plates one day before the experiment. Cells were left to adhere overnight. Peripheral blood mononuclear cells (PBMCs) were prepared by Histopaque (Sigma-Aldrich, Cat No. 10771-500ML Histopaque-1077) density centrifugation of enriched lymphocyte preparations of heparinized blood obtained from a Buffy Coat (“Blutspende Zirich”). The blood was diluted 1:2 with sterile DPBS and layered over Histopaque gradient (Sigma, # H8889). After centrifugation (450×g, 30 minutes, room temperature), the plasma above the PBMC-containing interphase was discarded and PBMCs transferred in a new falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (Cedex HiRes) and stored in RPMI1640 medium containing 10% FCS and 1% Glutamax (Gibco 35050061) at 37° C., in a humidified incubator until the assay was started. To measure T-Cell Activation (FIGS. 9A-9H), the FAP-ICOS molecules were added at the indicated concentrations (range of 0.11 pM-5000 pM in triplicates) and the CEACAM5-TCB was added at a fixed concentration (80 pM). As controls, wells containing only the TCB molecule or only the FAP-ICOS molecules were included. For T-Cell Proliferation and Differentiation (FIGS. 10A and 10B), CEACAM5-TCB was added with increasing concentrations (range of 1.28 pM-20000 pM in triplicates) and the FAP-ICOS molecules were added at a fixed concentration (1000 pM)

PBMCs were added to target cells and Fibroblasts to obtain a final E:T ratio of 5:1:1. T-Cell Activation was assessed after 48 h of incubation at 37° C., 5% CO₂ by flow cytometric analysis, using antibodies recognizing the T cell activation markers CD69 (early activation marker) and CD25 (late activation marker). T Cell Proliferation was assessed after 96 h of incubation at 37° C., 5% CO₂ by Flow cytometric analysis, using absolute Counts of CD4+ and CD8+ cells normalized to Counting Beads. T Cell Differentiation was assessed after 96 h of incubation at 37° C., 5% CO₂ by Flow cytometric analysis, using antibodies against CD45RO and CCR7 to discriminate the memory subsets.

Briefly, PBMCs were centrifuged at 400×g for 4 min and washed twice with PBS containing 0.1% BSA (FACS buffer). Surface staining for CD8 (PerCP/Cy5.5 anti-human CD8a, BioLegend #301032), CD4 (APC/Cy7 anti-human CD4, BioLegend #300518), CD69 (BV421 anti-human CD69, BioLegend #310930), CD25 (PE anti-human CD25, BioLegend #356104), CD45RO (APC anti-human CD45RO, BioLegend #304210) and CCR7 (FITC anti-human CCR7, BioLegend #353216) was performed according to the suppliers' indications. Absolute T-Cell Counts were obtained using Counting Beads (Precision Count Beads, BioLegend #424902) according to manufacturer's instruction. Cells were then washed twice with 150 μl/well PBS containing 0.1% BSA and fixed for 15-30 min at 4° C. using 75 μl/well FACS buffer, containing 1% PFA. After centrifugation, the samples were re-suspended in 150 μl/well FACS buffer and analyzed using BD FACS Fortessa.

### Example 13

### Primary Murine Splenocyte Co-Culture Assay

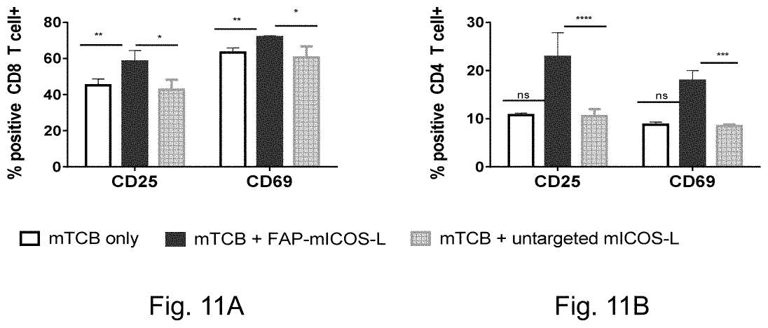

T-cell activation induced by mCEA-TCB upon co-incubation of murine splenocyte effector cells and human CEA-expressing MC38-hCEA target cells (parental cell line as described in Cancer Res 1975, 35:2434-2440, 3T3modified in-house to stably overexpress human CEA) in the presence or absence of mICOS-L molecules was assessed as follows (FIGS. 11A and 11B):

1.25 million splenocytes isolated from spleens of hCEA(HO) transgenic C57/BL6 mice or C57/BL6 wildtype mice (6.5) were added to 25 000 pre-plated MC38-hCEA tumor target cells per well of a flat-bottom-96-well plate. A fixed concentration of 1.5 nM of mCEA-TCB and 50 nM of a FAP-targeted or an untargeted DP47-mlCOS-L molecule was added and the assay plate was incubated for 48 h at 37° C. and 5% CO₂ in a humidified incubator in T cell media (RPMI1640, containing 10% FCS, 1× GlutaMax (Gibco #35050061), 50 mM×β-Mercaptoethanol (Sigma, # M3148-100), 200 U/ml IL2 (Proleukin, Novartis), 1× antibiotic-antimycotic (Gibco #15240062), 1 mM Sodium Pyruvate (Gibco #11360070)).

### Example 14

### In Vivo Functional Characterization of FAP-Targeted ICOS Molecule in Combination with CEACAM5-TCB

14.1 Pharmacokinetic Profile of FAP-ICOS (1+1) after Single Injection in NSG Mice

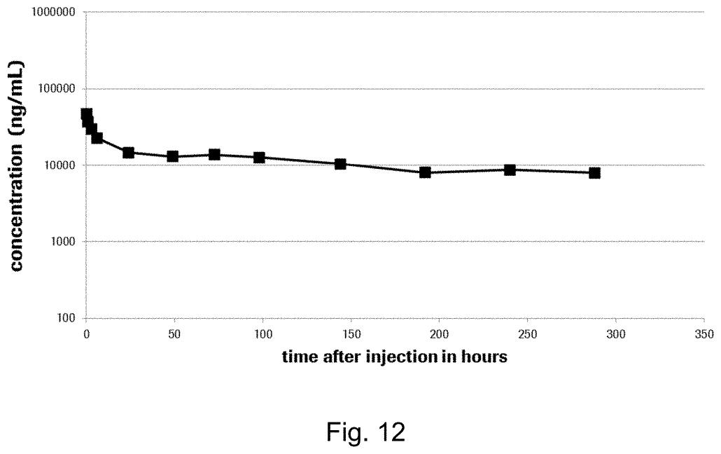

A single dose of 2.5 mg/kg of FAP-ICOS (1+1) was injected into NSG mice. All mice were injected i.v. with 200 μl of the appropriate solution. To obtain the proper amount of compounds per 200 μl, the stock solution (Table 12, FAP-ICOS) was diluted with histidine buffer. Three mice per time point were bled at 10 min, 1 hr, 3 hr, 6 hr, 24 hr, 48 hr, 72 hr, 96 hr, 6 days, 8 days, 10 days and 12 days. The injected compound was analyzed in serum samples by ELISA. Detection of the molecule was carried out by huIgG ELISA (detection via human Fc Antibody part). The plate was washed three times after each step to remove unbound substances. Finally, the peroxidase-bound complex was visualized by adding ABTS substrate solution to form a colored reaction product. The reaction product intensity which was photometrically determined at 405 nm (with reference wavelength at 490 nm) was proportional to the analyte concentration in the serum sample. The result (FIG. 12) showed a stable PK-behavior (T_{1/2}: 9.52 days) which suggested a once weekly schedule for subsequent efficacy studies.

TABLE 13
Description of tested composition

| | | Concentration |
|---|---|---|
| Compound | Formulation buffer | (mg/mL) |
| FAP-ICOS (1 + 1) | 20 mM Histidine, | 2.74 |
| | 140 mM NaCl, | (=stock solution) |
| | pH 6.0 | |

14.2 In Vivo Efficacy of FAP-ICOS (1+1) in Combination with CEACAM5-TCB in MKN45 Cografted with 3T3-huFAP in Fully Humanized NOG Mice

The first proof of concept study for the combination of FAP-ICOS and CEACAM5-TCB was aimed to understand the potency in terms of tumor regression and Immuno-PD in fully humanized NSG mice.

Human MKN45 cells (human gastric carcinoma) were originally obtained from ATCC and after expansion deposited in the Glycart internal cell bank. Cells were cultured in DMEM containing 10% FCS at 37° C. in a water-saturated atmosphere at 5% CO₂. In vitro passage 12 was used for subcutaneous injection at a viability of 97%. Human fibroblasts NIH-3T3 were originally obtained from ATCC (ATCC # CCL-92), engineered at Roche Nutley to express human FAP and cultured in DMEM containing 10% Calf serum, 1× Sodium Pyruvate and 1.5 μg/ml Puromycin. Clone 39 was used at an in vitro passage number 18 and at a viability of 98.2%.

50 microliters cell suspension (1×106 MKN45 cells+1×10⁶ 3T3-huFAP) mixed with 50 microliters Matrigel were injected subcutaneously in the flank of anaesthetized mice with a 22G to 30G needle.

Female NSG mice, age 4-5 weeks at start of the experiment (Jackson Laboratory) were maintained under specific-pathogen-free condition with daily cycles of 12 h light/12 h darkness according to committed guidelines (GV-Solas; Felasa; TierschG). The experimental study protocol was reviewed and approved by local government (P 2011/128). After arrival, animals were maintained for one week to get accustomed to the new environment and for observation. Continuous health monitoring was carried out on a regular basis.

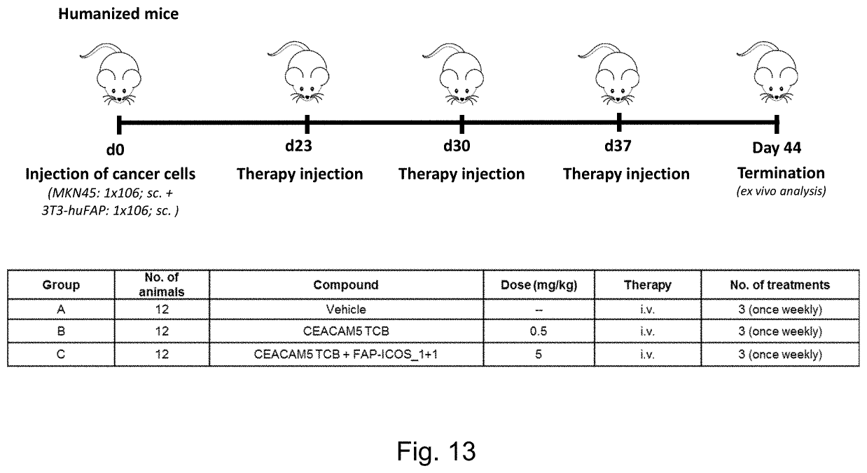

Female NSG mice were injected i.p. with 15 mg/kg of Busulfan followed one day later by an i.v. injection of 1×105 human hematopoietic stem cells isolated from cord blood. At week 14-16 after stem cell injection mice were bled sublingual and blood was analyzed by flow cytometry for successful humanization. Efficiently engrafted mice were randomized according to their human T cell frequencies into the different treatment groups. At that time, mice were injected with tumor cells and fibroblasts s.c. as described (FIG. 13) and treated once weekly with the compounds or Histidine buffer (Vehicle) when tumor size reached appr. 200 mm³ (day 23). All mice were injected i.v. with 200 l of the appropriate solution. To obtain the proper amount of compounds per 200 μl, the stock solutions (Table 13) were diluted with Histidine buffer when necessary. For combination therapy (Group C, FIG. 13) with FAP-ICOS and CEACAM5-TCB the respective compositions were injected concomitant. Tumor growth was measured twice weekly using a caliper (FIG. 2) and tumor volume was calculated as followed:

Tᵥ: (W²/2)×L (W: Width, L: Length)

At termination (day 44), mice were sacrificed, tumors and spleen were removed, weighted and single cell suspensions were prepared through an enzymatic digestion with Collagenase V, Dispase II and DNAse for subsequent FACS-analysis. Single cells where stained for human CD45, CD3, CD8, CD4, CD25 and FoxP3 (intracellular) and analyzed at FACS Fortessa.

Small pieces (30 mg) of tumor tissues were snap frozen and whole protein was isolated. Protein suspensions were analysed for cytokine content by Multiplex analysis.

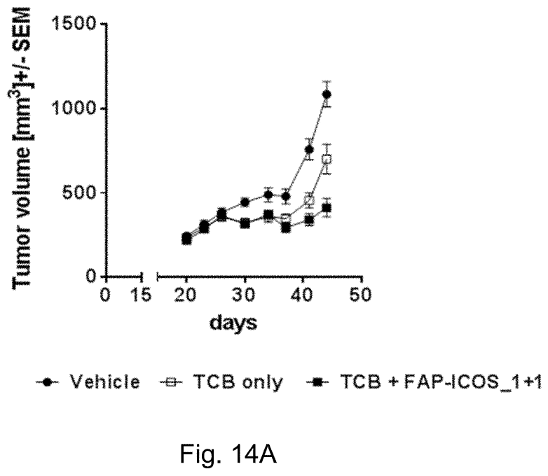

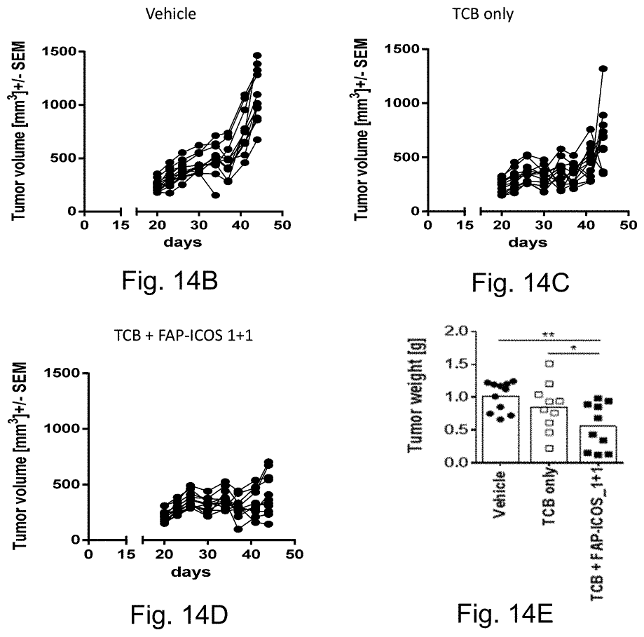

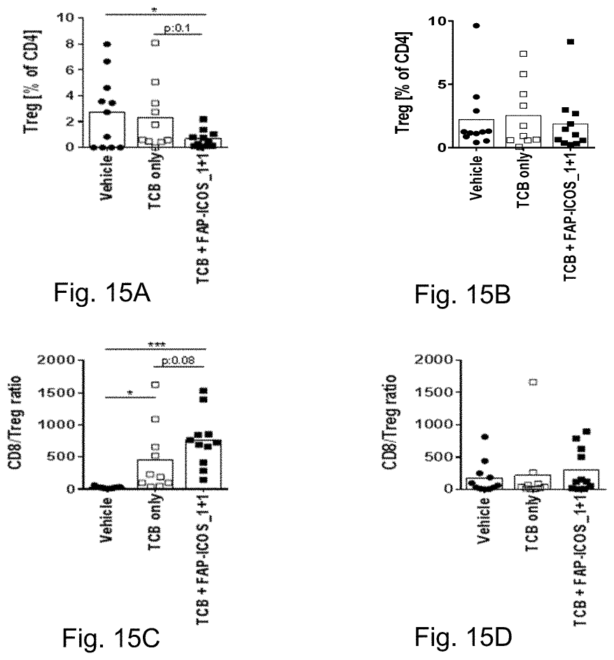

FIGS. 14A-14E show the tumor growth kinetics (Mean) in all treatment groups as well as the tumor weights at study termination. CEACAM5-TCB as a single agent induced some tumor growth inhibition. However, the combination with FAP-ICOS showed significant improved tumor growth inhibition that was also reflected by tumor weight at study termination. Interestingly, the Immuno-PD data (FIGS. 15A-15D) of tumors from animals sacrificed at study termination, revealed a depletion of human Treg cells specifically in the tumor tissue. No depletion was detected in spleen of treated animals (FIGS. 15C and 15D, flow cytometry). The depletion of Treg shifted the CD8/Treg towards CD8 cells in the combination treatment.

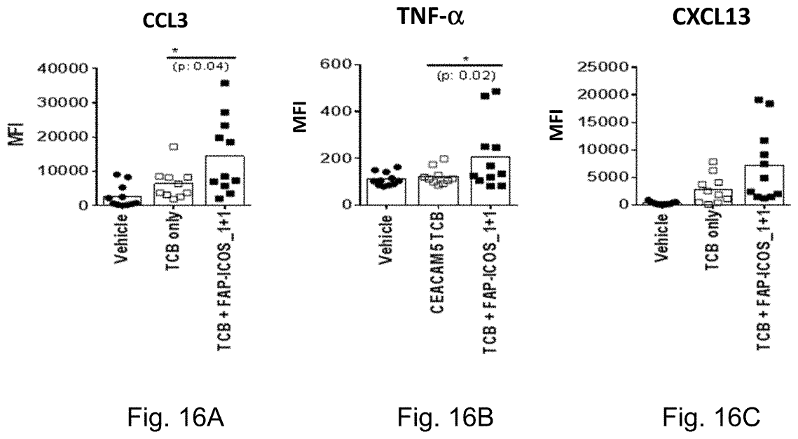

Furthermore, the cytokine analyses showed evaluated levels of CXCL13, TNF-α and CCL3 within the tumor tissue in the combination group (FIGS. 16A, 16B and 16C).

TABLE 14
Description of tested composition

| | | Concentration |
|---|---|---|
| Compound | Formulation buffer | (mg/mL) |
| FAP-ICOS (1 + 1) | 20 mM Histidine, | 2.74 |
| | 140 mM NaCl, | (=stock solution) |
| | pH 6.0 | |
| CEACAM 5 TCB | 20 mM Histidine, | 4.7 |
| | 140 mM NaCl, | (=stock solution) |
| | pH 6.0 | |

### Example 15

### Gene Expression Analysis

To identify ICOS-regulated genes, tumour tissues of the above efficacy study in humanized NSG mice (Example 14) were analyzed using the nCounter® Human Immunology Panel (NanoString Technologies, Seattle, USA).

Briefly, fresh frozen humanized mouse tumour tissues (30-70 mg) were ruptured and homogenized in RNA lysis buffer (Qiagen, Hilden, Germany), followed by total RNA extraction using the RNEasy Mini Kit (Qiagen, Hilden, Germany). Gene expression was quantified using the NanoString nCounter platform.

200 ng of total RNA was analyzed with the nCounter® Human Immunology Panel (NanoString Technologies, Seattle, USA) comprising a panel of barcoded probes matching 594 different immunology-related human genes. The codeset was hybridized with the RNA over night at 65° C. RNA transcripts were immobilized and barcoded probes were counted using the NanoString nCounter Digital Analyzer. Normalized raw expression data (nSolver Analysis software) were analyzed when 2 standard deviations above the geometric mean of the codeset-internal negative control probes were reached. Genes were excluded from further analysis if 90% of their expression was below the background threshold. The genes that remained after background-filtering were normalized to the geometric mean of the internal positive controls as well as to 5 housekeeping genes (GAPDH, HPRT1, ALAS1, GUSB and TUBB) and then log 2-transformed.

Data was analyzed with Qlucore Omics Explorer (QOE) software (Qlucore AB, Lund, Sweden). A PCA and hierarchical clustering were performed to determine the differences in gene expression between the treatment groups. Genes were considered differentially expressed when displaying a 2-fold difference in transcript level.

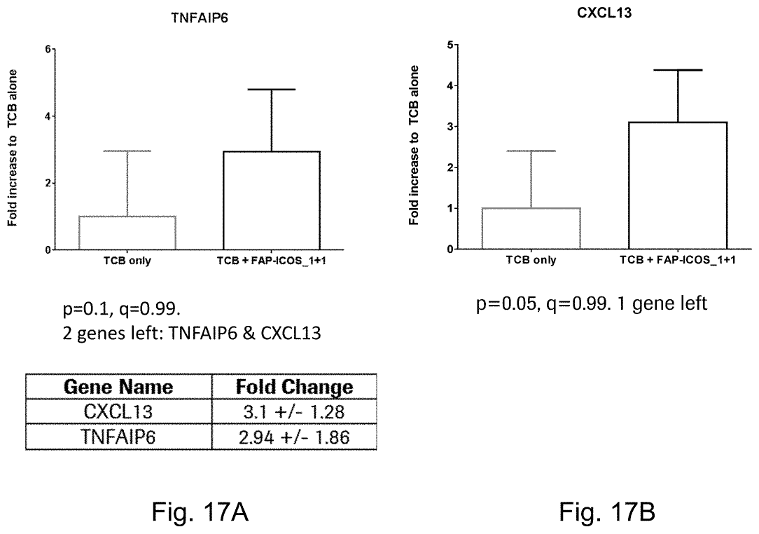

FIGS. 17A and 17B show TNFAIP6 and CXCL13 being the two strongest ICOS-up-regulated genes in combination with CEACAM5-TCB-mediated T-cell activation.

### Results

### 1. In-Vitro Characterization of FAP-ICOS Molecules

To understand a potential clinical relevance of ICOS as a cancer immunotherapeutic target, the expression of ICOS on TILs of various tumour indications was assessed.

As depicted in FIGS. 3A-3D, tumor-infiltrating-lymphocytes of both subtypes, CD4⁺ and CD8⁺ T-cells, show on average higher ICOS expression as T-cells from the same tumour patients isolated from normal tissue, or (peripheral) T-cells from healthy donors. Significant donor variations in expression level, as well as percent of ICOS-expressing T-cells could be observed. Upon ex vivo co-incubation of patient-derived tumour pieces with TILs, Lovo tumor cells and CEACAM5-TCB, the ICOS level on TILs could be increased only marginally, since most of the T-cells were already ICOS-positive at baseline (FIGS. 3E-3H).

However, co-incubation of Lovo tumor cells with CEACAM5-TCB and freshly isolated PBMCs of healthy donors shows much lower baseline levels of ICOS on CD4⁺ and CD8⁺ T-cells and a profound increase of ICOS upon TCB-mediated activation of T-cells.

The up-regulation of ICOS on both, CD4⁺ and CD8⁺ T-cells, directly correlates with the TCB concentration used for the T-cell activation and is seen consistently with different TCB molecules (FIGS. 4A to 4D).

Likewise, the co-incubation of mCEA-TCB or mCEACAM5-TCB with CEA-positive tumor cells and CD3-positive T-cells is able to up-regulate the expression of murine ICOS on CD4+, CD8+ and Treg cells in a concentration-dependent manner (FIGS. 5A-5F).

This validates ICOS as a therapeutic target being up-regulated at baseline already for a lot of tumour indications, as well as a potential combination partner for TCBs in settings with low ICOS baseline expression on T-cells.

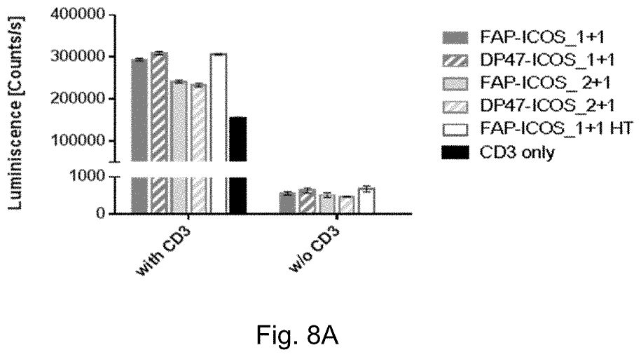

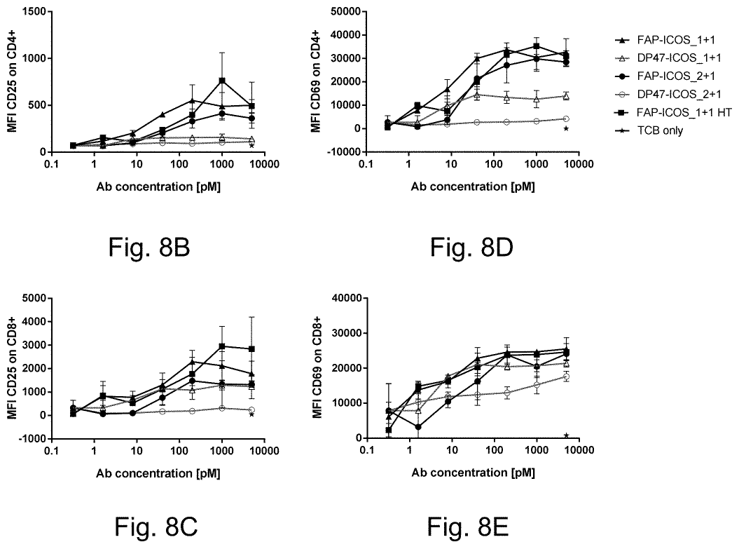

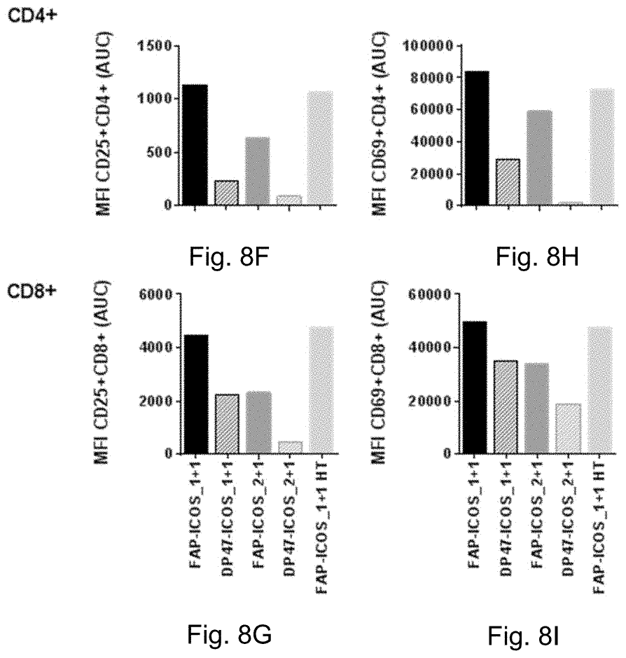

In vitro cell binding assays verify that the ICOS-FAP molecules are able to bind to both human FAP as well as human ICOS on cells in a concentration dependent manner (FIGS. 6A and 6B: Binding to hICOS on human T-Cells; FIG. 6C: Binding to huFAP on 3T3-huFAP cells). As expected, the untargeted ICOS-DP47 control molecules show no binding to huFAP (FIG. 8B). The molecules that target FAP with a c-terminal fusion (ICOS-FAP 2+1/ICOS-FAP 1+1 HT) show a slightly higher EC50 value for binding to human FAP (Table 10). No significant differences between the formats can be found for the binding to human ICOS.

As depicted in FIGS. 7A and 7B, both molecules, the untargeted DP47-mFAP-L, as well as the FAP-mICOSL bind to murine ICOS in a concentration dependent manner. In addition, FAP-mICOS-L shows concentration-dependent binding to murine FAP, as expected.

Characterization of FAP-ICOS molecules using Jurkat-NFAT Reporter cells or primary cells demonstrated dependency of ICOS signaling on simultaneous CD3/TCR engagement (FIG. 8A). All ICOS molecules were crosslinked by direct binding to the plate and therefore, the DP47-ICOS and the FAP-ICOS molecules showed similar activities in the presence of a simultaneous CD3 trigger. However, in the absence of a simultaneous CD3 trigger, no downstream signaling could be observed.

In a second approach, the effect of targeting, respective crosslinking on the induction of ICOS signaling was assessed: cross-linking of the anti-ICOS antibodies via binding of their FAP moiety to human FAP expressing cells led to much more effective signaling downstream of ICOS, as compared to the untargeted DP47-ICOS molecules, which were assessed in solution (FIGS. 8B-8E and 8F-8I). It should be noted, that in this model system, also the untargeted molecules show a certain level of activity, however to a much lower extent than their respective targeted versions. This clearly indicates the importance of crosslinking to exploit full power of the ICOS signaling.

Given that ICOS is upregulated upon CEACAM5-TCB treatment (FIGS. 3A-3H and FIGS. 4A-4D) the potential of the FAP-ICOS molecules to enhance CEACAM5-TCB mediated T-Cell Activation was assessed (FIGS. 9A-9H). FIGS. 9A to 9D show exemplary graphs of enhanced CD25 and CD69 expression on CD4⁺ (9A and 9B) and CD8⁺ (9C and 9D) T-Cells, revealing a dose dependent increase in CD25 and CD69 expression above CEACAM5-TCB treatment only. FIGS. 9E-9H depict a summary of up to 5 different healthy human PBMC donors and all five FAP-ICOS or DP47-ICOS molecules. It should be noted that the enhancing effects of FAP-ICOS, as well as the baseline activation levels due to CEACAM5-TCB treatment vary quite strongly between different donors, with some donors not displaying an enhancing effect of FAP-ICOS at all (e.g. Donor 4).

In line with the observations above, the co-incubation of FAP-targeted mICOS-L molecules in combination with a mCEA-TCB molecule leads to enhanced T-cell activation as compared to the TCB effect alone, which could be seen in a dose-dependent up-regulation of CD25 and CD69 on CD4+ and CD8+ T-cells (FIGS. 11A and 11B).

To further characterize the effects of combining CEACAM5-TCB with a targeted ICOS therapy, we characterized the response of primary cells after 96 hours in terms of T-Cell proliferation and T-Cell differentiation (FIG. 10). As depicted, the combination of CEACAM5-TCB and FAP-ICOS induced proliferation of both, CD4⁺ and CD8⁺ T-cells as compared to CEACAM5-TCB treatment only (FIGS. 10A and 10B). All three formats perform comparably well. FIGS. 12C-12F and 12G-12J show that increased numbers of effector memory and central memory T-Cells upon combination treatment with CEACAM5-TCB and FAP-ICOS compared to CEACAM5-TCB monotherapy at increasing TCB concentrations were observed, respective at a fixed concentration of 2.9 pM of the CEACAM5-TCB (FIG. 10K), again all three formats performing equally well. This supports the finding, that the boosting effect on early T-Cell Activation (FIG. 9) propagates to an increase in total number of T-Cells and an increase in the formation of desired memory T-Cell Subsets, which are supposed to enable long-term efficient anti-tumor responses in patients.

### 2. In-Vivo Characterization of FAP-ICOS Molecules

The FAP-ICOS_1+1 molecule from the screening assays above was evaluated in vivo using humanized NSG mice, co-grafted with human CEA-expressing MKN45 tumor cells and 3T3-hFAP expressing fibroblasts. As expected, the monotherapy of CEACAM5-TCB led to a delay in tumor growth. However, the combination of CEACAM5-TCB and FAP-ICOS induced a much stronger anti-tumor response, which was due to a tumor-specific Treg depletion, activation of T effector cells and consequently an improved ratio of CD8 effector to Treg cells.

Moreover, cytokine analysis of remaining tumor samples, revealed significantly up-regulated levels of CXCL13, TNF-alpha and CCL3 in the combination group compared to the monotherapy with CEACAM5-TCB (FIGS. 16A to 16C).

CCL3 is a cytokine belonging to the CC chemokine family and has been shown to interact with CCL4. It is also known as MIP1alpha and is described to attract macrophages, monocytes and neutrophils. In the tumor microenvironment CCL3 augments the antitumor immune response in a vaccine-context (Allen, 2016).

In line with the cytokine analysis above, CXCL13 and TNFAIP6 were identified as the two strongest ICOS-specific regulated genes (FIGS. 17A and 17B).

Tumor necrosis factor-inducible gene 6 protein also known as TNF-stimulated gene 6 protein or TSG-6. It is described as a potential biomarker of disease activity in inflammatory bowel disease. It exhibits a hyaluronan-binding domain that is known to be involved in extracellular matrix stability and cell migration.

The chemokine (C—X—C motif) ligand 13 (CXCL13) is also known as B lymphocyte chemoattractant (BLC) or B cell-attracting chemokine 1 (BCA-1). CXCR5(hi)ICOS(hi) CD4 T cells are the most potent inducers of IgG production that also secrete large amounts of the B cell-attracting chemokine CXCL13. Therefore, CXCL13 and its receptor CXCR5 control the organization of B cells within follicles of lymphoid tissues and might play an important role in the formation of tertiary lymphoid structures in several tumor indications.

The B cell chemoattractant CXCL13 has recently been linked with T_{FH} cell infiltration and improved survival in human cancer, due to local memory B cell differentiation induced by CXCL13-producing (CXCR5) follicular helper T cells (Gu-Trantien 2017). This again highlights a potential important role of ICOS in mediating potent anti-tumor responses via different secondary mechanisms.

Taken together, ICOS may serve as a potent anti-tumor molecule as monotherapy in several inflamed tumor indications with significant ICOS baseline expression of tumor-responsive T-cells. Using a FAP-targeted ICOS molecule allows tumor-specific activation of ICOS-expressing activated T effector cells and therefore is supposed to have a preferential safety and potency profile as compared to untargeted ICOS molecules.

T-cell bispecifics are potent immune engangers, which can induce T-cell activation, as well as enhance T-cell infiltration into tumours. Elevated ICOS expression upon TCB therapy could enable the targeting of a much broader patient population as compared to ICOS monotherapy approaches by combining a potent TCB with a tumor-specific ICOS molecule, which is expected to lead to enhanced and prolonged anti-tumor responses compared to a TCB or ICOS monotherapy.

### Example 16

### Generation of ICOS Antibodies

### 16.1 Preparation, Purification and Characterization of Antigens and Screening Tools for the Generation of Novel Antibodies by Phage Display

### 16.1.1 Preparation, Purification and Characterization of Dimeric Murine ICOS Antigen Fc(kih) Fusion Molecules

DNA sequences encoding the ectodomain of murine ICOS (Table 15) were subcloned in frame with the human IgG1 heavy chain CH2 and CH3 domains on the hole and knob for dimeric ICOS antigen Fc fusion molecules (Merchant et al., 1998). An Avi tag for directed biotinylation was introduced at the C-terminus of the antigen-Fc knob chain. Combination of the antigen-Fc knob chain containing the S354C/T366W mutations, with a Fc hole chain containing the Y349C/T366S/L368A/Y407V mutations allows generation of a ICOS heterodimer which includes 2 copies of the ectodomain containing chain, thus creating a dimeric form of Fc-linked antigen. Table 16 shows the cDNA and amino acid sequences of the antigen Fc-fusion construct.

TABLE 15
Amino acid numbering of antigen
ectodomain (ECD) and their origin

| SEQ ID NO: | Construct | Origin | ECD |
|---|---|---|---|
| 118 | murine ICOS ECD | Synthetized according to | aa 21-144 |
| | | Q9WVS0 | |

TABLE 16
cDNA and amino acid sequences of dimeric antigen
Fc(kih) fusion molecules

| SEQ ID NO: | Antigen | Sequence |
|---|---|---|
| 119 | Nucleotide | GAGATCAACGGCAGCGCCGACCACCGGATGTTCAGCTTC |
| | sequence | CACAATGGCGGCGTGCAGATCAGCTGCAAGTACCCCGAG |
| | murine ICOS | ACAGTGCAGCAGCTGAAGATGCGGCTGTTCCGCGAGCGG |
| | antigen | GAAGTGCTGTGCGAGCTGACCAAGACAAAGGGCAGCGGC |
| | Fc hole chain | AACGCCGTGTCCATCAAGAACCCCATGCTGTGCCTGTACC |
| | | ACCTGAGCAACAACAGCGTGTCCTTCTTCCTGAACAACCC |
| | | CGACAGCAGCCAGGGCAGCTACTACTTCTGCTCCCTGAGC |
| | | ATCTTCGACCCCCCACCATTCCAGGAACGGAACCTGAGCG |
| | | GCGGCTACCTGCACATCTACGAGAGCCAGCTGTGCTGCCA |
| | | GCTGAAACTGTGGCTGTCTGCAGACGTCGACGACAAAAC |
| | | TCACACATGCCCACCGTGCCCAGCACCTGAAGCTGCAGG |
| | | GGGACCGTCAGTCTTCCTCTTCCCCCCAAAACCCAAGGAC |
| | | ACCCTCATGATCTCCCGGACCCCTGAGGTCACATGCGTGG |
| | | TGGTGGACGTGAGCCACGAAGACCCTGAGGTCAAGTTCA |
| | | ACTGGTACGTGGACGGCGTGGAGGTGCATAATGCCAAGA |
| | | CAAAGCCGCGGGAGGAGCAGTACAACAGCACGTACCGTG |
| | | TGGTCAGCGTCCTCACCGTCCTGCACCAGGACTGGCTGAA |
| | | TGGCAAGGAGTACAAGTGCAAGGTCTCCAACAAAGCCCT |
| | | CGGCGCCCCCATCGAGAAAACCATCTCCAAAGCCAAAGG |
| | | GCAGCCCCGAGAACCACAGGTGTGCACCCTGCCCCCATC |
| | | CCGGGATGAGCTGACCAAGAACCAGGTCAGCCTCTCGTG |
| | | CGCAGTCAAAGGCTTCTATCCCAGCGACATCGCCGTGGA |
| | | GTGGGAGAGCAATGGGCAGCCGGAGAACAACTACAAGA |
| | | CCACGCCTCCCGTGCTGGACTCCGACGGCTCCTTCTTCCT |
| | | CGTGAGCAAGCTCACCGTGGACAAGAGCAGGTGGCAGCA |
| | | GGGGAACGTCTTCTCATGCTCCGTGATGCATGAGGCTCTG |
| | | CACAACCGCTTCACGCAGAAGAGCCTCTCCCTGTCTCCGG |
| | | GTAAATGA |
| 120 | Nucleotide | GAGATCAACGGCAGCGCCGACCACCGGATGTTCAGCTTC |
| | sequence | CACAATGGCGGCGTGCAGATCAGCTGCAAGTACCCCGAG |
| | murine ICOS | ACAGTGCAGCAGCTGAAGATGCGGCTGTTCCGCGAGCGG |
| | antigen Fc | GAAGTGCTGTGCGAGCTGACCAAGACAAAGGGCAGCGGC |
| | knob chain | AACGCCGTGTCCATCAAGAACCCCATGCTGTGCCTGTACC |
| | | ACCTGAGCAACAACAGCGTGTCCTTCTTCCTGAACAACCC |
| | | CGACAGCAGCCAGGGCAGCTACTACTTCTGCTCCCTGAGC |
| | | ATCTTCGACCCCCCACCATTCCAGGAACGGAACCTGAGCG |
| | | GCGGCTACCTGCACATCTACGAGAGCCAGCTGTGCTGCCA |
| | | GCTGAAACTGTGGCTGTCTGCAGACGTCGACGCTAGCGGT |
| | | GGTAGTCCGACACCTCCGACACCCGGGGGTGGTTCTGCA |
| | | GACAAAACTCACACATGCCCACCGTGCCCAGCACCTGAA |
| | | GCCGCAGGGGGACCGTCAGTCTTCCTCTTCCCCCCAAAAC |
| | | CCAAGGACACCCTCATGATCTCCCGGACCCCTGAGGTCAC |
| | | ATGCGTGGTGGTGGACGTGAGCCACGAAGACCCTGAGGT |
| | | CAAGTTCAACTGGTACGTGGACGGCGTGGAGGTGCATAA |
| | | TGCCAAGACAAAGCCGCGGGAGGAGCAGTACAACAGCAC |
| | | GTACCGTGTGGTCAGC GTCCTCACCGTCCTGCACCAGGAC |
| | | TGGCTGAATGGCAAGGAGTACAAGTGCAAGGTCTCCAAC |
| | | AAAGCCCTCGGAGCCCCCATCGAGAAAACCATCTCCAAA |
| | | GCCAAAGGGCAGCCCCGAGAACCACAGGTGTACACCCTG |
| | | CCCCCATGCCGGGATGAGCTGACCAAGAACCAGGTCAGC |
| | | CTGTGGTGCCTGGTCAAAGGCTTCTATCCCAGCGACATCG |
| | | CCGTGGAGTGGGAGAGCAATGGGCAGCCGGAGAACAACT |
| | | ACAAGACCACGCCTCCCGTGCTGGACTCCGACGGCTCCTT |
| | | CTTCCTCTACAGCAAGCTCACCGTGGACAAGAGCAGGTG |
| | | GCAGCAGGGGAACGTCTTCTCATGCTCCGTGATGCATGAG |
| | | GCTCTGCACAACCACTACACGCAGAAGAGCCTCTCCCTGT |
| | | CTCCGGGTAAATCCGGAGGCCTGAACGACATCTTCGAGG |
| | | CCCAGAAGATTGAATGGCACGAGTGA |
| 121 | murine ICOS | EINGSADHRMFSFHNGGVQISCKYPETVQQLKMRLFREREV |
| | antigen Fc hole | LCELTKTKGSGNAVSIKNPMLCLYHLSNNSVSFFLNNPDSSQ |
| | chain | GSYYFCSLSIFDPPPFQERNLSGGYLHIYESQLCCQLKLWLSA |
| | | DVDDKTHTCPPCPAPEAAGGPSVFLFPPKPKDTLMISRTPEV |
| | | TCVVVDVSHEDPEVKFNWYVDGVEVHNAKTKPREEQYNST |
| | | YRVVSVLTVLHQDWLNGKEYKCKVSNKALGAPIEKTISKA |
| | | KGQPREPQVCTLPPSRDELTKNQVSLSCAVKGFYPSDIAVE |
| | | WESNGQPENNYKTTPPVLDSDGSFFLVSKLTVDKSRWQQG |
| | | NVFSCSVMHEALHNRFTQKSLSLSPGK |
| 122 | murine ICOS | EINGSADHRMFSFHNGGVQISCKYPETVQQLKMRLFREREV |
| | antigen Fc | LCELTKTKGSGNAVSIKNPMLCLYHLSNNSVSFFLNNPDSSQ |
| | knob chain | GSYYFCSLSIFDPPPFQERNLSGGYLHIYESQLCCQLKLWLSA |
| | | DVDASGGSPTPPTPGGGSADKTHTCPPCPAPEAAGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALGAPIEKTISKAKGQPREPQVYTLPPCRDELTKNQVSLW |
| | | CLVKGFYPSDIAVEWESNGQPENNYKTTPPVLDSDGSFFLYS |
| | | KLTVDKSRWQQGNVFSCSVMHEALHNHYTQKSLSLSPGKS |
| | | GGLNDIFEAQKIEWHE |

The ICOS-Fc-fusion encoding sequences were cloned into a plasmid vector driving expression of the insert from a chimeric MPSV promoter and containing a synthetic polyA signal sequence located at the 3′ end of the CDS. In addition, the vector contained an EBV OriP sequence for episomal maintenance of the plasmid. For preparation of the biotinylated antigen/Fc fusion molecules, exponentially growing suspension HEK293 EBNA cells were co-transfected with three vectors encoding the two components of fusion protein (knob and hole chains) as well as BirA, an enzyme necessary for the biotinylation reaction. The corresponding vectors were used at a 1:1:0.05 ratio (“Fc knob”:“Fc hole”:“BirA”).

For protein production in 500 ml shake flasks, 400 million HEK293 EBNA cells were seeded 24 hours before transfection. For transfection cells were centrifuged for 5 minutes at 210 g, and supernatant was replaced by pre-warmed CD CHO medium. Expression vectors were resuspended in 20 mL of CD CHO medium containing 200 μg of vector DNA. After addition of 540 μL of polyethylenimine (PEI), the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL of F17 medium was added and cells were cultured for 24 hours. One day after transfection, 1 mM valproic acid and 7% Feed were added to the culture. After 7 days of culturing, the cell supernatant was collected by spinning down cells for 15 min at 210 g. The solution was sterile filtered (0.22 μm filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a HiTrap ProteinA HP column (CV=5 mL, GE Healthcare) equilibrated with 40 mL 20 mM sodium phosphate, 20 mM sodium citrate pH 7.5. Unbound protein was removed by washing with at least 10 column volumes of a buffer containing 20 mM sodium phosphate, 20 mM sodium citrate and 0.5 M sodium chloride (pH 7.5). The bound protein was eluted using a linear pH-gradient of sodium chloride (from 0 to 500 mM) created over 20 column volumes of 20 mM sodium citrate, 0.01% (v/v) Tween-20, pH 3.0. The column was then washed with 10 column volumes of a solution containing 20 mM sodium citrate, 500 mM sodium chloride and 0.01% (v/v) Tween-20, pH 3.0. The pH of the collected fractions was adjusted by adding 1/40 (v/v) of 2M Tris, pH8.0. The protein was concentrated and filtered prior to loading on a HiLoad Superdex 200 column (GE Healthcare) equilibrated with 2 mM MOPS, 150 mM sodium chloride, 0.02% (w/v) sodium azide solution of pH 7.4.

### 16.1.2 Generation and Characterisation of a Stable Cell Line Expressing Recombinant Murine ICOS

Full-length cDNA encoding murine ICOS was subcloned into mammalian expression vector. Plasmids were transfected into CHO-K1 (ATCC, CCL-61) cells using Lipofectamine LTX Reagent (Invitrogen, #15338100) according to the manufacturer's protocol. Stably transfected ICOS-positive CHO-K1 cells were maintained in DMEM/F-12 (Gibco, #11320033) supplemented with 10% fetal bovine serum (Gibco, #16140063) and 1% GlutaMAX Supplement (Gibco; #31331-028). Two days after transfection, puromycin (Invivogen; # ant-pr-1) was added to 6 μg/mL. After initial selection, the cells with the highest cell surface expression of ICOS were sorted using BD FACSAria III cell sorter (BD Biosciences) and cultured to establish stable cell clones. The expression level and stability was confirmed by FACS analysis using PE anti-human/mouse/rat CD278 antibody (BioLegend; #313508) over a period of 4 weeks.

### 16.2 Generation of ICOS-Specific 16E09 Antibody (Surrogate Molecule) by Phage Display

16.2.1 Selection of ICOS-Specific Antibodies from Generic Fab Libraries

For the selection of clones binding to murine ICOS recombinant antigen the selection approaches by phage display was performed. Prior to selection rounds pre-clearing step was included to get rid of undesired binders, for example, to the tags. For this purpose 4 wells of neutravidin plate (Pierce, Cat. No. 15128) were coated with 200 μl of 100 nM solution of biotinylated antigen (depleter) in PBS for 30 min at 37° C. and washed briefly with PBS.

600 μl of library phages (produced inhouse) were incubated with 200 μl 5% BSA in PBS for 10 min on an orbital shaker, at room temperature. Pre-blocked library mixture was transferred to the 4 wells coated with biotinylated depleter and incubated for 1 hour at room temperature. The supernatant was carefully removed from the wells and used for selection.

The selection was executed in three rounds on biotinylated ICOS-Fc fusion protein. For all selection rounds 30 phage display libraries were used (all libraries generated inhouse).

Panning rounds were performed in solution according to the following pattern: (1) binding of ˜10e12 pre-cleared phagemid particles to 100 nM biotinylated ICOS-Fc protein for 0.5 h in a total volume of 1 ml, (2) capture of biotinylated ICOS-Fc protein and specifically bound phage particles by addition of 5.4×10⁷ streptavidin-coated magnetic beads for 10 min, (3) washing of beads using 5×1 ml PBS/0.1%-Tween-20 and 5×1 ml PBS, (4) elution of phage particles by addition of 1 ml 100 mM TEA (Sigma-Aldrich, Cat. No. 90335) for 10 min and neutralization by adding 500 μl 1M Tris/HCl pH 7.4, (5) re-infection of exponentially growing E. coli TG1 bacteria, and (6) infection with helper phage VCSM13 and subsequent 20% PEG-2.5M NaCl precipitation of phagemid particles to be used in subsequent selection rounds. In round 2, capture of antigen: phage complexes were performed using neutravidin plates instead of streptavidin beads. Neutravidin plates were washed with 5×PBS/0.1%-Tween-20 and 5×PBS. Third selection round was carried out with decreased antigen concentrations of 20 nM.

16.2.2 Screening of Specific ICOS Clones after Selection Rounds

ICOS specific clones were identified in two different methods by Mirrorball. For Screening of ICOS specific clones on recombinant ICOS antigen in 384 well plate, 50 μl of Streptavidin coated Sol-RTM beads (TTP Labtech, Cat. No. 4150-09125) were coated with 80 nM of biotinylated antigen and incubated for 1 h at room temperature. Beads with bound antigen were washed 1× with PBS. The Antigen-beads complex was resuspended in 23 ml PBS. Alexa Fluor® 647 AffiniPure Goat anti-human IgG, F(ab′)₂ fragment specific antibodies (Cat. No. 109-605-006) were added to the mixture with the final concentration of 800 ng/ml. The mixture was equally distributed at the volume of 35 μl into each well. 5 μl of Fabs in supernatant, was added to each well. Samples were incubated for 2 hours at room temperature prior to reading.

For Screening of ICOS specific clones on recombinant cells expressing ICOS antigen in 384 well plates, cells were harvested by centrifugation and the supernatant was discarded. Cells were resuspended in pre-warmed CellTracker Green CMFDA (Invitrogen, Cat. No. C7025) working solution at a concentration of 1.2×10⁶ cells/ml followed by cell incubation under growth condition. The CellTracker Green CMFDA working solution was replaced with fresh, pre-warmed culture medium. Cells were incubated for another 30 minutes under growth condition followed by washing 2× with PBS. Cells were resuspended in total volume of 8 ml PBS. Alexa Fluor® 647 AffiniPure Goat anti-human IgG, F(ab′)2 fragment specific antibodies (Jackson ImmunoResearch, Cat. No. 109-605-006) were added with the final concentration of 800 ng/ml. The mixture was equally distributed at the volume of 20 μl into each well. 5 μl of Fabs in supernatant, with 15 μl of PBS, were added to each well. Samples were incubated for 2 hours at room temperature prior to reading.

### 16.2.3 Sequencing of Selected Clones

Clones that were Mirrorball-positive (on recombinant proteins and on recombinant cells expressing ICOS antigen) were chosen for the sequencing to reveal unique clones.

The sequences of selected clone 16E09 are shown in Table 17 below.

TABLE 17
Variable region amino acid sequences for phage-derived
anti-murine ICOS antibody 16E09. Underlined are the
complementarity determining regions (CDRs).

| | SEQ ID | |
|---|---|---|
| Clone | NO: | Sequence |
| 16E09 | 130 (VL) | DIVMTQSPLSLPVTPGEPASISCRSSQSLLHSNGYNYLDWYLQKPGQSP |
| | | QLLIYLGSNRASGVPDRFSGSGSGTDFTLKISRVEAEDVGVYYCMQAL |
| | | WTPTTFGQGTKVEIK |
| | 129 (VH) | EVQLVQSGAEVKKPGESLKISCKGSGYSFTSYWIGWVRQMPGKGLEW |
| | | MGIIYPGDSDTRYSPSFQGQVTISADKSISTAYLQWSSLKASDTAMYYC |
| | | ARSSGPYGLYLDYWGQGTLVTVSS |

### 16.2.4 Fab Expression and Purification of Unique Clones

The expression of a monoclonal antibody Fab fragment in TG1 cells was induced by 1 mM isopropyl-beta-D-thiogalactoside (IPTG), in Difco 2×YT medium (BD, Cat. No. 244020) with addition of 100 ug/ml ampicillin (AppliChem, Cat. No. A0839.0100), overnight, at 30° C. Purification of Fabs was performed on His GraviTrap affinity columns according to the manufacturer's protocol (GE Healthcare, Cat. No. 11-0033-99). Buffer exchange was performed on PD-10 Desalting Columns (GE Healthcare, Cat. No. 17-0851-01) according to the manufacturer's protocol.

### 16.2.5 Binding Characterization of Purified Clones on ProteOn XPR36 Instrument (Biorad)

The Affinity (K_{D}) of the monoclonal antibody Fab fragments was measured by SPR using a ProteOn XPR36 instrument (Biorad) at 25° C. For kinetics measurements, 15 μg/ml of murine ICOS proteins, as well as Fc-depleter, were injected. For one-shot kinetics measurements, dilution series of purified monoclonal antibody Fab fragments were injected simultaneously along separate channels 1-5. Buffer (PBST) was injected along the sixth channel to provide an “in-line” blank for referencing. Association rate constants (kₒₙ) and dissociation rate constants (k_{off}) were calculated using a simple one-to-one Langmuir binding model in ProteOn Manager v3.1 software by simultaneously fitting the association and dissociation sensorgrams. The equilibrium dissociation constant (K_{D}) was calculated as the ratio k_{off}/kₒₙ (Table 18).

TABLE 18
Kinetic equilibrium constants (K_{D}) of 16E09
anti-ICOS antibody as Fab fragment

| Clone | Murine ICOS |
|---|---|
| Fab fragment 16E09 | Affinity: 293 nM |

### 16.2.6 Binding Characterization of Purified Fab Fragment 16E09 on Recombinant Cells Expressing Murine ICOS

Binding of purified Fab fragment 16E09 was confirmed on stable CHO K1 cells expressing murine ICOS recombinant antigen.

### 16.3 Preparation and Purification of Anti-ICOS 16E09 IgG1 P329G LALA Antibody

The variable regions of heavy and light chain DNA sequences of the selected anti-ICOS clone were subcloned in frame with either the constant heavy chain or the constant light chain of human IgG1. The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

The amino acid sequences of the anti-ICOS clone are shown in Table 19. Anti-ICOS-Fc-fusion encoding sequences were cloned into a plasmid vector, which drives expression of the insert from an CMV promoter and contains a synthetic polyA signal sequence located at the 3′ end of the CDS.

TABLE 19
Amino acid sequences of anti-ICOS clone 16E09 in
P329GLALA human IgG1 format

| Clone | SEQ ID No. | Sequence |
|---|---|---|
| 16E09 | 131 | DIVMTQSPLSLPVTPGEPASISCRSSQSLLHSNGYNYLDWYLQ |
| | (Light chain) | KPGQSPQLLIYLGSNRASGVPDRFSGSGSGTDFTLKISRVEAE |
| | | DVGVYYCMQALWTPTTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| | 132 | EVQLVQSGAEVKKPGESLKISCKGSGYSFTSYWIGWVRQMPG |
| | (Heavy chain) | KGLEWMGIIYPGDSDTRYSPSFQGQVTISADKSISTAYLQWSS |
| | | LKASDTAMYYCARSSGPYGLYLDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHTCPPCPAPEAAGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALGAPIE |
| | | KTISKAKGQPREPQVYTLPPSRDELTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPG |

The anti-ICOS antibody was produced by co-transfecting HEK293-EBNA cells with the mammalian expression vectors using polyethylenimine. The cells were transfected with the corresponding expression vectors in a 1:1 ratio (“vector heavy chain”:“vector light chain”).

For production in 30 mL shake flasks, 60 million HEK293 EBNA cells were seeded before transfection. For transfection the cells were centrifuged for 10 minutes at 210×g, and the supernatant was replaced by pre-warmed CD CHO medium. Expression vectors (30 μg of total DNA) were mixed in 3 mL CD CHO medium. After addition of 81 μL PEI, the solution was vortexed for 15 seconds and incubated for 10 minutes at room temperature. Afterwards, cells were mixed with the DNA/PEI solution, transferred to a 50 mL shake flask and incubated for 3 hours at 37° C. in an incubator with a 5% CO₂ atmosphere. After the incubation, 24 mL of Excell medium supplemented with 6 mM L-Glutamine, 5 g/L PEPSOY medium and 1 mM valproic acid was added and cells were cultured for 24 hours. One day after transfection 12% Feed 7 and Glucose (final conc. 3 g/L) were added. After culturing for 7 days, the supernatant was collected by centrifugation for 30 minutes at 400×g. The solution was sterile filtered (0.22 μm filter), supplemented with sodium azide to a final concentration of 0.01% (w/v), and kept at 4° C.

Purification of antibody molecules from cell culture supernatants was carried out by affinity chromatography using Protein A as described above for purification of antigen Fc fusions. The protein was concentrated and filtered prior to loading on a HiLoad Superdex 200 column (GE Healthcare) equilibrated with 20 mM Histidine, 140 mM NaCl solution of pH 6.0.

The protein concentration of purified antibodies was determined by measuring the OD at 280 nm, using the molar extinction coefficient calculated on the basis of the amino acid sequence. Purity and molecular weight of the antibodies were analyzed by CE-SDS in the presence and absence of a reducing agent (Invitrogen, USA) using a LabChipGXII (Caliper). The aggregate content of antibody samples was analyzed using a TSKgel G3000 SW XL analytical size-exclusion column (Tosoh) equilibrated in a 25 mM K₂HPO₄, 125 mM NaCl, 200 mM L-Arginine Monohydrocloride, 0.02% (w/v) NaN₃, pH 6.7 running buffer at 25° C. Table 20 summarizes the yield and final content of the anti-ICOS IgG1 antibody.

TABLE 20
Biochemical analysis of anti-ICOS P329G LALA IgG1 antibody

| | Yield | Monomer | Purity [%] |
|---|---|---|---|
| Clone | [mg/l] | [%] aSEC | CE-SDS |
| 16E09 IgG1 P329GLALA | 2.63 | 98 | 98 |

### 16.4 Characterization of Anti-ICOS 16E09 IgG1 P329G LALA Antibody

### 16.4.1 Binding on Murine ICOS as Measured by Surface Plasmon Resonance (Avidity+Affinity)

Binding of the phage-derived ICOS-specific antibody to the recombinant monomeric ICOS Fc(kih) was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Kinetic constants were derived using the Biacore T200 Evaluation Software (vAA, Biacore AB, Uppsala/Sweden), to fit rate equations for 1:1 Langmuir binding by numerical integration and used to estimate qualitatively the avidity.

In the same experiment, the affinities of the interaction between the phage-derived ICOS-specific antibody 16E09 to recombinant murine ICOS was determined. For this purpose, the ectodomain of murine ICOS was subcloned in frame with an avi (GLNDIFEAQKIEWHE) tag (for the sequences see Table 21).

TABLE 21
Amino acid sequences of murine ICOS Fc(kih) Avi tag

| SEQ ID NO: | Antigen | Sequence |
|---|---|---|
| 133 | murine ICOS Fc | EINGSADHRMFSFHNGGVQISCKYPETVQQLKMRLFR |
| | knob Avi-tag | EREVLCELTKTKGSGNAVSIKNPMLCLYHLSNNSVSFF |
| | | LNNPDSSQGSYYFCSLSIFDPPPFQERNLSGGYLHIYES |
| | | QLCCQLKLWLSADVDASGGSPTPPTPGGGSADKTHTC |
| | | PPCPAPEAAGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALGAPIEKTISK |
| | | AKGQPREPQVYTLPPCRDELTKNQVSLWCLVKGFYPS |
| | | DIAVEWESNGQPENNYKTTPPVLDSDGSFFLYSKLTVD |
| | | KSRWQQGNVFSCSVMHEALHNHYTQKSLSLSPGKSG |
| | | GLNDIFEAQKIEWHE |
| 134 | murine ICOS Fc | EINGSADHRMFSFHNGGVQISCKYPETVQQLKMRLFR |
| | hole | EREVLCELTKTKGSGNAVSIKNPMLCLYHLSNNSVSFF |
| | | LNNPDSSQGSYYFCSLSIFDPPPFQERNLSGGYLHIYES |
| | | QLCCQLKLWLSADVDDKTHTCPPCPAPEAAGGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALGAPIEKTISKAKGQPREPQVCTLPP |
| | | SRDELTKNQVSLSCAVKGFYPSDIAVEWESNGQPENN |
| | | YKTTPPVLDSDGSFFLVSKLTVDKSRWQQGNVFSCSV |
| | | MHEALHNRFTQKSLSLSPGK |

Protein production was performed as described in Example 16.1 for the Fc-fusion protein. Secreted proteins were purified from cell culture supernatants by chelating chromatography, followed by size exclusion chromatography. The first chromatographic step was performed on a NiNTA Superflow Cartridge (5 ml, Qiagen) equilibrated in 20 mM sodium phosphate, 500 nM sodium chloride, pH7.4. Elution was performed by applying a gradient over 12 column volume from 5% to 45% of elution buffer (20 mM sodium phosphate, 500 nM sodium chloride, 500 mM Imidazole, pH7.4). The protein was concentrated and filtered prior to loading on a HiLoad Superdex 75 column (GE Healthcare) equilibrated with 2 mM MOPS, 150 mM sodium chloride, 0.02% (w/v) sodium azide solution of pH 7.4.

Binding of the ICOS specific antibody 16E09 to recombinant murine ICOS Fc(kih) was assessed by surface plasmon resonance as described above for human ICOS Fc(kih) (see Example 16.2.5). Surface plasmon resonance experiments were performed on a Biacore T200 at 25° C. with HBS-EP as running buffer (0.01 M HEPES pH 7.4, 0.15 M NaCl, 0.005% (v/v) Surfactant P20 (GE Healthcare)).

For affinity determination, murine ICOS Fc(kih) was directly coupled on a streptavidin chip using the standard manufacturer procedure (SA chip, GE Healthcare). The immobilization level was approximately 600 RU. Affinity of recombinant ICOS specific antibody was determined with multicycle kinetic by injection of six concentrations (1000 nM-31.25 nM, 1:2 dilution) at 30 l/min for 240 s to record the association phase. The dissociation phase was monitored for 500 s and triggered by switching from the sample solution to HBS-EP. The chip surface was regenerated after every cycle using one injection of 10 mM Glycine pH 3.0 for 60 s.

Bulk refractive index differences were corrected by subtracting the response obtained on reference flow cell, anti-ICOS antibodies antigens were flown over a surface where no ICOS receptor was immobilized.

The affinity constants were derived from the kinetic rate constants by fitting to a 1:1 Langmuir binding using the BIAeval software (GE Healthcare). It was shown that antibody 16E09 binds murine ICOS (Table 22) with high affinity.

TABLE 22
Binding of anti-ICOS antibody 16E09 to murine ICOS

| | | Recombinant murine ICOS (affinity format) | | |
|---|---|---|---|---|
| Clone | Origin | ka (1/Ms) | kd (1/s) | K_{D} (M) |
| 16E09 | Phage | 1.44E+05 | 1.92E−02 | 1.80E−07 |
| | display | | | |

### Example 17

### Generation of Bispecific Antibodies with a Monovalent or Bivalent Binding to Murine ICOS and a Monovalent Binding to FAP

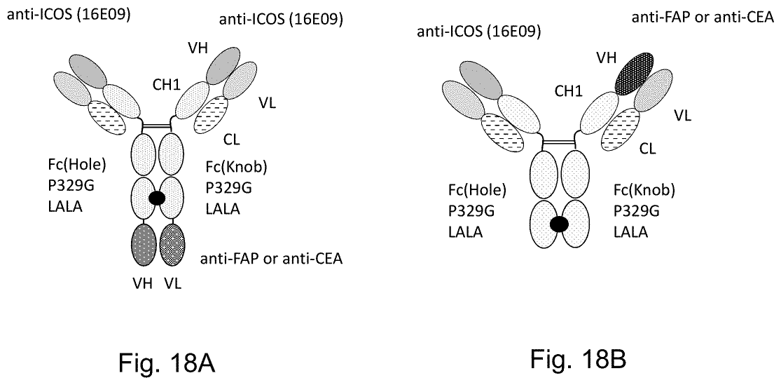

The following bispecific agonistic ICOS antibodies with monovalent or bivalent binding for ICOS and monovalent binding for FAP have been prepared as depicted in FIGS. 18A and 18B, respectively.

A) ICOS(16E09)-FAP(28H1) 2+1, FAP-targeted anti-murine ICOS_2+1, mIgG1 DAPG, bivalent murine ICOS (16E09), monovalent FAP (28H1) (FIG. 18A, SEQ ID Nos: 135, 136 and 131).

B) ICOS(16E09)-FAP(28H1) 1+1, FAP-targeted anti-murine ICOS_1+1, mIgG1 DAPG, monovalent murine ICOS (16E09), monovalent FAP (28H1) (FIG. 18B, SEQ ID Nos: 137, 138, 131 and 139).

For ICOS(16E09)-FAP(28H1) 2+1, the first heavy chain (HC1) of the FAP-targeted anti-murine ICOS_2+1 construct was comprised of the following components: VHCH1 of an anti-ICOS (16E09) on a murine Fc DAPG DD region at which C-terminus a VH of anti-FAP binder (28H1) was fused. The second heavy chain (HC2) was comprised of VHCH1 of an anti-ICOS (16E09) on a murine Fc DAPG KK region at which C-terminus a VL of anti-FAP binder (28H1) was fused.

The ‘DDKK’ knob-into-hole technology is described in e.g. in WO 2014/131694 A1, and Combination of the Fc DD with the Fc KK chain allows generation of a heterodimer. Briefly, aspartic acid residues (D) are provided in the Fc region subunit of one of the heavy chains (HCl) at positions corresponding to positions 392 and 409 (numbering according to Kabat EU index; i.e. K392D and K409D), and lysine (K) residues are provided in the Fc region subunit of the other of the heavy chains (HC2) at positions corresponding to positions 356 and 399 (numbering according to Kabat EU index; i.e. E356K and D399K). DAPG mutations are introduced in the constant regions of the heavy chains to abrogate binding to murine Fc gamma receptors according to the method described e.g. in Baudino et al. J. Immunol. (2008), 181, 6664-6669, or in WO 2016/030350 A1. Briefly, alanine (A) is provided in the Fc region at the position corresponding to position 265, and glycine (G) is provided in the Fc region at the position corresponding to position 329 (numbering according to Kabat EU index; i.e. D265A, P329G).

In the ICOS(16E09)-FAP(28H1) 1+1 HCl was comprised of VHCH1 of an anti-ICOS (16E09) on a murine Fc DAPG DD. HC2 was comprised of VHCH1 of anti-FAP (28H1) followed by Fc DAPG KK. For the murine ICOS binder, the VH and VL sequences were obtained from a phage display campaign as described in Example 16.2. The generation and preparation of the FAP binder (28H1) is described in WO 2012/020006 A2, which is incorporated herein by reference.

The amino acid sequences for bispecific agonistic ICOS antibodies can be found respectively in Tables 23 and 24 below.

TABLE 23
Amino acid sequences of bispecific ICOS(16E09)-FAP(28H1)
2 + 1 murine IgG1 DAPG

| SEQ | | |
|---|---|---|
| ID NO: | Description | Sequence |
| 135 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG DD | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VH (28H1) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKEQMAKDKVSLTCMITNFFPEDITVEWQWN |
| | | GQPAENYDNTQPIMDTDGSYFVYSDLNVQKSNWEAGNTFTC |
| | | SVLHEGLHNHHTEKSLSHSPGGGGGSGGGGSGGGGSGGGGS |
| | | EIVLTQSPGTLSLSPGERATLSCRASQSVSRSYLAWYQQKPGQ |
| | | APRLLIIGASTRATGIPDRFSGSGSGTDFTLTISRLEPEDFAVYY |
| | | CQQGQVIPPTFGQGTKVEIK |
| 136 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG KK | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VL (28H1) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKKQMAKDKVSLTCMITNFFPEDITVEWQWN |
| | | GQPAENYKNTQPIMKTDGSYFVYSKLNVQKSNWEAGNTFTC |
| | | SVLHEGLHNHHTEKSLSHSPGGGGGSGGGGSGGGGSGGGGS |
| | | EVQLLESGGGLVQPGGSLRLSCAASGFTFSSHAMSWVRQAP |
| | | GKGLEWVSAIWASGEQYYADSVKGRFTISRDNSKNTLYLQM |
| | | NSLRAEDTAVYYCAKGWLGNFDYWGQGTLVTVSS |
| 131 | VLCL | DIVMTQSPLSLPVTPGEPASISCRSSQSLLHSNGYNYLDWYLQ |
| | (16E09)-light | KPGQSPQLLIYLGSNRASGVPDRFSGSGSGTDFTLKISRVEAE |
| | chain | DVGVYYCMQALWTPTTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPIVK |
| | | SFNRNEC |

TABLE 24
Amino acid sequences of bispecific ICOS(16E09)-FAP(28H1) 1 + 1
murine IgG1 DAPG

| SEQ | | |
|---|---|---|
| ID NO: | Description | Sequence |
| 137 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG DD | SLKASDTAMYYCARSSGPYGLYLDYWGQGTLVTVSSAKTTP |
| | heavy chain | PSVYPLAPGSAAQTNSMVTLGCLVEGYFPEPVTVTWNSGSLS |
| | | SGVHTFPAVLQSDLYTLSSSVTVPSSTWPSQTVTCNVAHPASS |
| | | TKVDEKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKEQMAKDKVSLTCMITNFFPEDITVEWQWN |
| | | GQPAENYDNTQPIMDTDGSYFVYSDLNVQKSNWEAGNTFTC |
| | | SVLHEGLHNHHTEKSLSHSP |
| 138 | VHCH1 | EIVLTQSPGTLSLSPGERATLSCRASQSVSRSYLAWYQQKPGQ |
| | (28H1) Fc | APRLLIIGASTRATGIPDRFSGSGSGTDFTLTISRLEPEDFAVYY |
| | DAPG KK | CQQGQVIPPTFGQGTKVEIKSSAKTTPPSVYPLAPGSAAQTNS |
| | heavy chain | MVTLGCLVKGYFPEPVTVTWNSGSLSSGVHTFPAVLQSDLYT |
| | | LSSSVTVPSSTWPSQTVTCNVAHPASSTKVDKKIVPRDCGCK |
| | | PCICTVPEVSSVFIFPPKPKDVLTITLTPKVTCVVVAISKDDPEV |
| | | QFSWFVDDVEVHTAQTKPREEQINSTFRSVSELPIMHQDWLN |
| | | GKEFKCRVNSAAFGAPIEKTISKTKGRPKAPQVYTIPPPKKQM |
| | | AKDKVSLTCMITNFFPEDITVEWQWNGQPAENYKNTQPIMK |
| | | TDGSYFVYSKLNVQKSNWEAGNTFTCSVLHEGLHNHHTEKS |
| | | LSHSP |
| 131 | VLCL | See Table 23 |
| | (16E09)-light | |
| | chain | |
| 139 | VLCL (28H1)- | EVQLLESGGGLVQPGGSLRLSCAASGFTFSSHAMSWVRQAP |
| | light chain | GKGLEWVSAIWASGEQYYADSVKGRFTISRDNSKNTLYLQM |
| | | NSLRAEDTAVYYCAKGWLGNFDYWGQGTLVTVSSASDAAP |
| | | TVSIFPPSSEQLTSGGASVVCFLNNFYPKDINVKWKIDGSERQ |
| | | NGVLNSWTDQDSKDSTYSMSSTLTLTKDEYERHNSYTCEAT |
| | | HKTSTSPIVKSFNRNEC |

The corresponding cDNAs were cloned into evitria's vector system using conventional (non-PCR based) cloning techniques. The evitria vector plasmids were gene synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with up to two sequencing reactions per plasmid depending on the size of the cDNA).

Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 m filter).

Secreted proteins were purified from cell culture supernatants by affinity chromatography using Protein A, followed by size exclusion chromatography. For affinity chromatography, the supernatant was loaded on a Protein A MabSelectSure column (CV=5 mL, GE Healthcare) equilibrated with 40 mL 20 mM sodium phosphate, 20 mM sodium citrate pH 7.5. Unbound protein was removed by washing with at least 10 column volumes of 20 mM sodium phosphate, 20 mM sodium citrate containing buffer (pH 7.5). The bound protein was eluted using a linear pH-gradient of sodium chloride (from 20 to 100 mM) created over 15 column volumes of 20 mM sodium citrate, 100 mM NaCl, 100 mM Glycine, 0.01% Tween-20 pH 3.0. The column was then washed with 10 column volumes of 20 mM sodium citrate, 100 mM NaCl, 100 mM Glycine, 0.01% Tween-20 pH 3.0. The pH of collected fractions was adjusted by adding 1/40 (v/v) of 2M Tris, pH8.0. The protein was concentrated and filtered prior to loading on a HiLoad Superdex 50/600 S200 column (GE Healthcare) equilibrated with 20 mM Histidine, 140 mM NaCl, 0.01% Tween-20 pH6.0.

The protein concentration of purified bispecific constructs was determined by measuring the OD at 280 nm, using the molar extinction coefficient calculated on the basis of the amino acid sequence. Purity and molecular weight of the bispecific constructs were analyzed by CE-SDS in the presence and absence of a reducing agent (Invitrogen, USA) using a LabChipGXII (Caliper). The aggregate content of bispecific constructs was analyzed using a TSKgel G3000 SW XL analytical size-exclusion column (Tosoh) equilibrated in a 25 mM K₂HPO₄, 125 mM NaCl, 200 mM L-Arginine Monohydrochloride, 0.02% (w/v) NaN₃, pH 6.7 running buffer at 25° C.

Generation of Bispecific Antibodies with a Monovalent or Bivalent Binding to Murine ICOS and an Untargeted Moiety (Control Molecules)

The following bispecific agonistic ICOS antibodies with monovalent or bivalent binding for murine ICOS were prepared similarly to the targeted formats as depicted in FIGS. 18A and 18B, respectively, however instead of anti-FAP they comprise an untargeted moiety.

C) ICOS(16E09)-DP47 2+1, untargeted anti-murine ICOS 2+1, muIgG1 DAPG, bivalent murine ICOS (16E09), monovalent untargeted moiety (DP47) (SEQ ID Nos: 140, 141 and 131).

D) ICOS(16E09)-DP47 1+1, untargeted anti-murine ICOS 1+1, muIgG1 DAPG, monovalent murine ICOS (16E09), monovalent untargeted moiety DP47 (SEQ ID Nos: 142, 143, 131 and 144).

In example C, the untargeted anti-murine ICOS 2+1 construct was comprised of the following components: first heavy chain (HC1): VHCH1 of an anti-ICOS (16E09) followed by murine Fc DAPG DD, at which C-terminus a VH of a non-binding clone (DP47) was fused. Second heavy chain (HC2) was comprised of VHCH1 of an anti-ICOS (16E09) followed by murine Fc DAPG KK, at which C-terminus a VL of a non-binding clone (DP47) was fused.

The untargeted anti-murine ICOS 1+1 construct was comprised of the following components: HCl was comprised of VHCH1 of anti-ICOS (16E09) followed by murine Fc DAPG DD. HC2 was comprised of VHCH1 of DP47 as non-binding antibody followed by a murine Fc DAPG KK.

The amino acid sequences for the untargeted agonistic ICOS antibodies can be found respectively in Tables 25 and 26.

TABLE 25
Amino acid sequences of bispecific 2 + 1 untargeted DP47
anti-ICOS(16E09) murine IgG1 DAPG

| SEQ | | |
|---|---|---|
| ID NO: | Description | Sequence |
| 140 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG DD | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VH (DP47) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKEQMAKDKVSLTCMITNFFPEDITVEWQWN |
| | | GQPAENYDNTQPIMDTDGSYFVYSDLNVQKSNWEAGNTFTC |
| | | SVLHEGLHNHHTEKSLSHSPGGGGGSGGGGSGGGGSGGGGS |
| | | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQYGSSPLTFGQGTKVEIK |
| 141 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG KK | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VL (DP47) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKKQMAKDKVSLTCMITNFFPEDITVEWQWN |
| | | GQPAENYKNTQPIMKTDGSYFVYSKLNVQKSNWEAGNTFTC |
| | | SVLHEGLHNHHTEKSLSHSPGGGGGSGGGGSGGGGSGGGGS |
| | | EVQLLESGGGLVQPGGSLRLSCAASGFTFSSYAMSWVRQAP |
| | | GKGLEWVSAISGSGGSTYYADSVKGRFTISRDNSKNTLYLQM |
| | | NSLRAEDTAVYYCAKGSGFDYWGQGTLVTVSS |
| 131 | VLCL | See Table 23 |
| | (16E09)-light | |
| | chain | |

TABLE 26
Amino acid sequences of bispecific 1 + 1 untargeted DP47
anti-ICOS(16E09) murine IgG1 DAPG

| SEQ | | |
|---|---|---|
| ID NO: | Description | Sequence |
| 142 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG DD | SLKASDTAMYYCARSSGPYGLYLDYWGQGTLVTVSSAKTTP |
| | heavy chain | PSVYPLAPGSAAQTNSMVTLGCLVEGYFPEPVTVTWNSGSLS |
| | | SGVHTFPAVLQSDLYTLSSSVTVPSSTWPSQTVTCNVAHPASS |
| | | TKVDEKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKEQMAKDKVSLTCMITNFFPEDITVEWQWN |
| | | GQPAENYDNTQPIMDTDGSYFVYSDLNVQKSNWEAGNTFTC |
| | | SVLHEGLHNHHTEKSLSHSP |
| 143 | VHCH1 | EVQLLESGGGLVQPGGSLRLSCAASGFTFSSYAMSWVRQAP |
| | (DP47) Fc | GKGLEWVSAISGSGGSTYYADSVKGRFTISRDNSKNTLYLQM |
| | DAPG KK | NSLRAEDTAVYYCAKGSGFDYWGQGTLVTVSSASDAAPTVS |
| | heavy chain | IFPPSSEQLTSGGASVVCFLNNFYPKDINVKWKIDGSERQNGV |
| | | LNSWTDQDSKDSTYSMSSTLTLTKDEYERHNSYTCEATHKTS |
| | | TSPIVKSFNRNECGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKKQMAKDKVSLTCMITNFFPEDITVEWQWN |
| | | GQPAENYKNTQPIMKTDGSYFVYSKLNVQKSNWEAGNTFTC |
| | | SVLHEGLHNHHTEKSLSHSP |
| 131 | VLCL | See Table 23 |
| | (16E09)-light | |
| | chain | |
| 144 | Murine VLCL | EIVLTQSPGTLSLSPGERATLSCRASQSVSSSYLAWYQQKPGQ |
| | (DP47)-light | APRLLIYGASSRATGIPDRFSGSGSGTDFTLTISRLEPEDFAVY |
| | chain | YCQQYGSSPLTFGQGTKVEIKSSAKTTPPSVYPLAPGSAAQTN |
| | | SMVTLGCLVKGYFPEPVTVTWNSGSLSSGVHTFPAVLQSDLY |
| | | TLSSSVTVPSSTWPSQTVTCNVAHPASSTKVDKKIVPRDC |

The untargeted bispecific agonistic ICOS molecules were prepared as described herein before for the FAP(28H1)-targeted bispecific agonistic ICOS antibodies.

The results of the biochemical analysis of the bispecific molecules with a monovalent or bivalent binding to murine ICOS (16E09) and a monovalent binding to FAP (28H1) or DP47 produced as described herein are summarized in Table 27.

TABLE 27
Biochemical analysis of bispecific
FAP-ICOS or DP47-ICOS molecules

| | | | CE-SDS |
|---|---|---|---|
| | | | (non- |
| | Monomer | Yield | reduced) |
| Molecule | [%] | [mg/L] | [%] |
| ICOS(16E09)-FAP(28H1) 2 + 1 | 100 | 2.58 | 100 |
| muIgG1 DAPG | | | |
| ICOS(16E09)-FAP(28H1) 1 + 1 | 100 | 2.15 | 100 |
| muIgG1 DAPG | | | |
| ICOS(16E09)-DP47 2 + 1 muIgG1 | 100 | 2.36 | 95.5 |
| DAPG | | | |
| ICOS(16E09)-DP47 1 + 1 muIgG1 | 100 | 2.15 | 100 |
| DAPG | | | |

### Example 18

### Generation of Bispecific Antibodies with a Monovalent or Bivalent Binding to ICOS and a Monovalent Binding to CEA

The following bispecific agonistic ICOS antibodies with monovalent or bivalent binding for ICOS and monovalent binding for CEA have been prepared as depicted in FIGS. 18A and 18B, respectively.

A) ICOS(JMab136)-CEA(MEDI-565) 2+1, CEA-ICOS_2+1 huIgG1 P329G LALA, bivalent ICOS (JMAb136), monovalent CEA (MEDI-565) (FIG. 18A, SEQ ID Nos: 153, 154 and 29).

B) ICOS(JMab136)-CEA(MEDI-565) 1+1, CEA-ICOS_1+1, huIgG1 P329G LALA, monovalent ICOS (JMAb136), monovalent CEA (MEDI-565) (FIG. 18B, SEQ ID Nos: 155, 29, 156 and 157).

The CEA-ICOS_2+1 construct was comprised of the following components: HC1 was built from VHCH1 of an anti-ICOS (JMAb136) followed by Fc hole, at which C-terminus a VH of anti-CEA binder (MEDI-565) was fused. HC2 was comprised of VHCH1 of anti-ICOS (JMAb136) followed by Fc knob, at which C-terminus a VL of anti-CEA binder (MEDI-565) was fused.

In the CEA-ICOS_1+1 construct, the HC1 was comprised of the following components, VHCH1 of an anti-ICOS (JMAb136) followed by Fc hole. HC2 was comprised of VHCH1 of anti-CEA (MEDI-565) followed by Fc knob.

For the ICOS binder, the VH and VL sequences of clone JMAb136 were obtained from patent US 2008/0199466 A1. For the CEA binder, the VH and VL sequences of clone MEDI-565 were obtained from patent WO 2014/079886 A1.

Combination of the Fc knob with the Fc hole chain allows generation of a heterodimer. The Pro329Gly, Leu234Ala and Leu235Ala mutations have been introduced in the constant region of the knob and hole heavy chains to abrogate binding to Fcγ receptor according to the method described in International Patent Appl. Publ. No. WO2012/130831A1.

The amino acid sequences for bispecific agonistic ICOS antibodies can be found in Tables 28 and 29, respectively.

TABLE 28
Amino acid sequences of bispecific 2 + 1 CEA(MEDI-565)-targeted
anti-ICOS(JMAb136) human IgG1 P329G LALA

| SEQ | | |
|---|---|---|
| ID NO: | Description | Sequence |
| 29 | VLCL | See Table 1 |
| | (JMAb136) | |
| | Light chain | |
| 153 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | knob chain-VH | MELSRLRSDDTAVYYCARTYYYDSSGYYHDAFDIWGQGTM |
| | (MEDI-565) | VTVSSASTKGPSVFPLAPSSKSTSGGTAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVYTLPPCRDELTKNQVS |
| | | LWCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGSGGGGSQAVLTQPASLSASPGASASLTCT |
| | | LRRGINVGAYSIYWYQQKPGSPPQYLLRYKSDSDKQQGSGVS |
| | | SRFSASKDASANAGILLISGLQSEDEADYYCMIWHSGASAVF |
| | | GGGTKLTVL |
| 154 | VHCH1 | QVQLVQSGAEVKKPGASVKVSCKASGYTFTGYYMHWVRQA |
| | (JMAb136)-Fc | PGQGLEWMGWINPHSGGTNYAQKFQGRVTMTRDTSISTAY |
| | hole chain-VL | MELSRLRSDDTAVYYCARTYYYDSSGYYHDAFDIWGQGTM |
| | (MEDI-565) | VTVSSASTKGPSVFPLAPSSKSTSGGTAALGCLVKDYFPEPVT |
| | | VSWNSGALTSGVHTFPAVLQSSGLYSLSSVVTVPSSSLGTQT |
| | | YICNVNHKPSNTKVDKKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVCTLPPSRDELTKNQVS |
| | | LSCAVKGFYPSDIAVEWESNGQPENNYKTTPPVLDSDGSFFL |
| | | VSKLTVDKSRWQQGNVFSCSVMHEALHNHYTQKSLSLSPGG |
| | | GGGSGGGGSEVQLVESGGGLVQPGRSLRLSCAASGFTVSSY |
| | | WMHWVRQAPGKGLEWVGFIRNKANGGTTEYAASVKGRFTI |
| | | SRDDSKNTLYLQMNSLRAEDTAVYYCARDRGLRFYFDYWG |
| | | QGTTVTVSS |

TABLE 29
Amino acid sequences of bispecific 1 + 1 CEA (MEDI-565)-targeted
anti-ICOS(JMAb136) human IgG1 P329G LALA

| SEQ | | |
|---|---|---|
| ID NO: | Description | Sequence |
| 155 | VHCH1(JMAb | QVQLVQSGAEVKKPGASVKVSCKASGYTFTGYYMHWVRQA |
| | 136)- Fc hole | PGQGLEWMGWINPHSGGTNYAQKFQGRVTMTRDTSISTAY |
| | chain | MELSRLRSDDTAVYYCARTYYYDSSGYYHDAFDIWGQGTM |
| | | VTVSSASTKGPSVFPLAPSSKSTSGGTAALGCLVEDYFPEPVT |
| | | VSWNSGALTSGVHTFPAVLQSSGLYSLSSVVTVPSSSLGTQT |
| | | YICNVNHKPSNTKVDEKVEPKSCDKTHTCPPCPAPEAAGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALGAPIEKTISKAKGQPREPQVCTLPPSRDELTKNQVS |
| | | LSCAVKGFYPSDIAVEWESNGQPENNYKTTPPVLDSDGSFFL |
| | | VSKLTVDKSRWQQGNVFSCSVMHEALHNHYTQKSLSLSP |
| 29 | VLCL(JMAb1 | DIQMTQSPSSVSASVGDRVTITCRASQGISRLLAWYQQKPGK |
| | 36) Light chain | APKLLIYVASSLQSGVPSRFSGSGSGTDFTLTISSLQPEDFATY |
| | | YCQQANSFPWTFGQGTKVEIKRTVAAPSVFIFPPSDRKLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGEC |
| 156 | VHCH1 | QAVLTQPASLSASPGASASLTCTLRRGINVGAYSIYWYQQKP |
| | (MEDI-565)- | GSPPQYLLRYKSDSDKQQGSGVSSRFSASKDASANAGILLISG |
| | Fc knob chain | LQSEDEADYYCMIWHSGASAVFGGGTKLTVLSSASTKGPSVF |
| | | PLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCDKTHTCPPCPAPEAAGGPSVFLFPPKPKDTLMIS |
| | | RTPEVTCVVVDVSHEDPEVKFNWYVDGVEVHNAKTKPREE |
| | | QYNSTYRVVSVLTVLHQDWLNGKEYKCKVSNKALGAPIEKT |
| | | ISKAKGQPREPQVYTLPPCRDELTKNQVSLWCLVKGFYPSDI |
| | | AVEWESNGQPENNYKTTPPVLDSDGSFFLYSKLTVDKSRWQ |
| | | QGNVFSCSVMHEALHNHYTQKSLSLSP |
| 157 | VLCL(MEDI- | EVQLVESGGGLVQPGRSLRLSCAASGFTVSSYWMHWVRQAP |
| | 565) Light | GKGLEWVGFIRNKANGGTTEYAASVKGRFTISRDDSKNTLYL |
| | chain | QMNSLRAEDTAVYYCARDRGLRFYFDYWGQGTTVTVSSAS |
| | | VAAPSVFIFPPSDEQLKSGTASVVCLLNNFYPREAKVQWKVD |
| | | NALQSGNSQESVTEQDSKDSTYSLSSTLTLSKADYEKHKVYA |
| | | CEVTHQGLSSPVTKSFNRGEC |

The bispecific agonistic ICOS antibodies with monovalent or bivalent binding for ICOS and monovalent binding for CEA were prepared as described in Example 17 for the FAP(28H1)-targeted bispecific agonistic ICOS antibodies. The results of the biochemical analysis of the bispecific molecules is summarized in Table 30 below.

TABLE 30
Biochemical analysis of bispecific
CEA-ICOS or DP47-ICOS molecules

| | | | CE-SDS |
|---|---|---|---|
| | | | (non- |
| | Monomer | Yield | reduced) |
| Molecule | [%] | [mg/L] | [%] |
| CEA(MEDI565)- | 94 | 4.55 | 91 |
| ICOS(JMAb136)_1 + 1 | | | |
| CEA(MEDI565)- | 97 | 3.41 | 97 |
| ICOS(JMAb136)_2 + 1 | | | |

Generation of Bispecific Antibodies with a Monovalent or Bivalent Binding to Murine ICOS and a Monovalent Binding to CEA

The following bispecific agonistic ICOS antibodies with monovalent or bivalent binding for murine ICOS and monovalent binding for CEA have been prepared as depicted in FIGS. 18A and 18B, respectively.

C) ICOS(16E09)-CEA(A5B7) 2+1 muIgG1 DAPG, CEA-targeted anti-murine ICOS_2+1, muIgG1 DAPG, bivalent murine ICOS (16E09), monovalent CEA (A5B7) (SEQ ID Nos: 166, 167 and 131).

D) ICOS(16E09)-CEA(A5B7) 1+1 muIgG1 DAPG, CEA-targeted anti-murine ICOS_1+1, muIgG1 DAPG, monovalent murine ICOS (16E09), monovalent CEA (A5B7) (SEQ ID Nos: 137, 131, 168 and 169).

In Example C, the HC1 of the CEA-targeted anti-murine ICOS_2+1 construct was comprised of the following components: VHCH1 of an anti-ICOS (16E09) on a murine Fc DAPG DD at which C-terminus a VL of anti-CEA binder (A5B7) was fused. HC2 was comprised of VHCH1 of an anti-ICOS (16E09) on a murine Fc DAPG KK at which C-terminus a VH of anti-CEA binder (A5B7) was fused.

The CEA-ICOS_1+1_construct (Example D) was comprised of HCl, VHCH1 of an anti-ICOS (16E09) on a murine Fc DAPG DD. HC2 was comprised of VHCH1 of anti-CEA (A5B7) followed by Fc DAPG KK.

Combination of the Fc DD with the Fc KK chain allows generation of a heterodimer. DAPG mutations are introduced in the constant regions of the heavy chains to abrogate binding to murine Fc gamma receptors according to the method described e.g. in Baudino et al. J. Immunol. (2008), 181, 6664-6669, or in WO 2016/030350 A1.

For the murine ICOS binder, the VH and VL sequences were obtained from a phage display campaign (Example 16.2). The generation and preparation of the CEA binder (A5B7) is described in WO 92/01059 which is incorporated herein by reference.

The amino acid sequences for bispecific agonistic ICOS antibodies can be found in Tables 31 and 32, respectively.

TABLE 31
Amino acid sequences of bispecific 2 + 1 CEA(A5B7)-targeted
anti-ICOS(16E09) murine IgG1 DAPG

| SEQ | | |
|---|---|---|
| ID NO: | Description | Sequence |
| 166 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG DD | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VL (A5B7) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKEQMAKDKVSLTCMITNFFPEDITVEWQWN |
| | | GQPAENYDNTQPIMDTDGSYFVYSDLNVQKSNWEAGNTFTC |
| | | SVLHEGLHNHHTEKSLSHSPGGGGGSGGGGSGGGGSGGGGS |
| | | QTVLSQSPAILSASPGEKVTMTCRASSSVTYIHWYQQKPGSSP |
| | | KSWIYATSNLASGVPARFSGSGSGTSYSLTISRVEAEDAATYY |
| | | CQHWSSKPPTFGGGTKLEIK |
| 167 | VHCH1 | EVQLVQSGAEVKKPGESLKISCKGSGYSFTSYWIGWVRQMP |
| | (16E09) Fc | GKGLEWMGIIYPGDSDTRYSPSFQGQVTISADKSISTAYLQWS |
| | DAPG KK | SLKASDTAMYYCARSSGPYGLYLDYWGQGTLVTVSSAKTTP |
| | heavy chain- | PSVYPLAPGSAAQTNSMVTLGCLVKGYFPEPVTVTWNSGSLS |
| | VH (A5B7) | SGVHTFPAVLQSDLYTLSSSVTVPSSTWPSQTVTCNVAHPASS |
| | | TKVDKKIVPRDCGCKPCICTVPEVSSVFIFPPKPKDVLTITLTP |
| | | KVTCVVVAISKDDPEVQFSWFVDDVEVHTAQTKPREEQINST |
| | | FRSVSELPIMHQDWLNGKEFKCRVNSAAFGAPIEKTISKTKGR |
| | | PKAPQVYTIPPPKKQMAKDKVSLTCMITNFFPEDITVEWQWN |
| | | GQPAENYKNTQPIMKTDGSYFVYSKLNVQKSNWEAGNTFTC |
| | | SVLHEGLHNHHTEKSLSHSPGGGGGSGGGGSGGGGSGGGGS |
| | | EVKLVESGGGLVQPGGSLRLSCATSGFTFTDYYMNWVRQPP |
| | | GKALEWLGFIGNKANGYTTEYSASVKGRFTISRDKSQSILYL |
| | | QMNTLRAEDSATYYCTRDRGLRFYFDYWGQGTTLTVSS |
| 131 | VLCL | See Table 19 |
| | (16E09)-light | |
| | chain | |

TABLE 32
Amino acid sequences of bispecific 1 + 1 CEA(A5B7)-targeted
anti-ICOS(16E09) murine IgG1 DAPG

| SEQ | | |
|---|---|---|
| ID NO: | Description | Sequence |
| 137 | VHCH1 | See Table 24 |
| | (16E09) Fc | |
| | DAPG DD | |
| | heavy chain | |
| 168 | VHCH1 | EVKLVESGGGLVQPGGSLRLSCATSGFTFTDYYMNWVRQPP |
| | (A5B7) Fc | GKALEWLGFIGNKANGYTTEYSASVKGRFTISRDKSQSILYL |
| | DAPG KK | QMNTLRAEDSATYYCTRDRGLRFYFDYWGQGTTLTVSSASD |
| | heavy chain | AAPTVSIFPPSSEQLTSGGASVVCFLNNFYPKDINVKWKIDGS |
| | | ERQNGVLNSWTDQDSKDSTYSMSSTLTLTKDEYERHNSYTC |
| | | EATHKTSTSPIVKSFNRNECGCKPCICTVPEVSSVFIFPPKPKD |
| | | VLTITLTPKVTCVVVAISKDDPEVQFSWFVDDVEVHTAQTKP |
| | | REEQINSTFRSVSELPIMHQDWLNGKEFKCRVNSAAFGAPIEK |
| | | TISKTKGRPKAPQVYTIPPPKKQMAKDKVSLTCMITNFFPEDI |
| | | TVEWQWNGQPAENYKNTQPIMKTDGSYFVYSKLNVQKSNW |
| | | EAGNTFTCSVLHEGLHNHHTEKSLSHSP |
| 131 | VLCL | See Table 19 |
| | (16E09)-light | |
| | chain | |
| 169 | VLCL (A5B7)- | QTVLSQSPAILSASPGEKVTMTCRASSSVTYIHWYQQKPGSSP |
| | light chain | KSWIYATSNLASGVPARFSGSGSGTSYSLTISRVEAEDAATYY |
| | | CQHWSSKPPTFGGGTKLEIKSSAKTTPPSVYPLAPGSAAQTNS |
| | | MVTLGCLVKGYFPEPVTVTWNSGSLSSGVHTFPAVLQSDLYT |
| | | LSSSVTVPSSTWPSQTVTCNVAHPASSTKVDKKIVPRDC |

The bispecific agonistic ICOS antibodies with monovalent or bivalent binding for murine ICOS and monovalent binding for CEA were prepared as described herein before for the FAP(28H1)-targeted bispecific agonistic ICOS antibodies. The results of the biochemical analysis of the bispecific molecules are summarized in Table 33.

TABLE 33
Biochemical analysis of bispecific CEA-ICOS molecules

| | | | CE-SDS |
|---|---|---|---|
| | | | (non- |
| | Monomer | Yield | reduced) |
| Molecule | [%] | [mg/L] | [%] |
| ICOS(16E09)-CEA(A5B7) 2 + 1 | 100 | 3.1 | 96.1 |
| muIgG1 DAPG | | | |
| ICOS(16E09)-CEA(A5B7) 1 + 1 | 100 | 5.97 | 97.4 |
| muIgG1 DAPG | | | |

### Example 19

### Binding of Bispecific Agonistic ICOS Antibodies to Cells Expressing Either Human ICOS or Human CEA (Flow Cytometry Analysis)

The binding of several bispecific agonistic ICOS antibodies as prepared in Example 18 was tested using ICOS-positive pre-activated PBMCs or CEA-positive MKN45 cells (human gastric adenocarcinoma cell line, DSMZ ACC 409). Briefly, human PBMCs, isolated from either Buffy Coats or fresh blood from healthy human donors (as described in Example 5.1) were incubated in a humidified incubator at 37° C. for 48 hours in RPMI1640 medium including 2 g/ml PHA-L (Roche, #11249738001) and 100 units/ml recombinant human IL-2 (“Proleukin”, Novartis) at a cell density of 1 million cells per ml.

At the day of the assay, pre-activated PBMCs cells were harvested, washed with PBS, and 0.2 million cells in FACS buffer (PBS with 0.1% BSA) were plated per well of a 96-round bottom-well plate. Likewise, adherent MKN45 tumor cells were detached using trypsin (Gibco, 25300-054), washed and 0.15 million cells were transferred into the 96-round bottom well-plate. Subsequent staining was performed for 30 min at 4° C. with increasing concentrations of the anti-ICOS (7 pM-120 nM). Thereafter, cells were washed twice with cold PBS 0.1% BSA and further incubated for further 30 min at 4° C. with a labeled secondary antibody (Alexa Fluor 647-conjugated AffiniPure F(ab′)₂ Fragment Goat Anti-Human IgG, Fcγ fragment specific (190-606-008) from Jackson Immuno Research Lab, diluted 1 to 100 in FACS buffer). Cells were washed with FACS buffer and the staining was fixed for 20 min at 4° C. in the dark, using 75 l of 1% PFA in FACS buffer per well. Cells were washed again two times with FACS Buffer and re-suspended in FACS Buffer for analysis.

Fluorescence was analyzed by FACS using a FACS Fortessa (Software FACS Diva). Binding curves and EC₅₀ values were obtained using GraphPadPrism6.

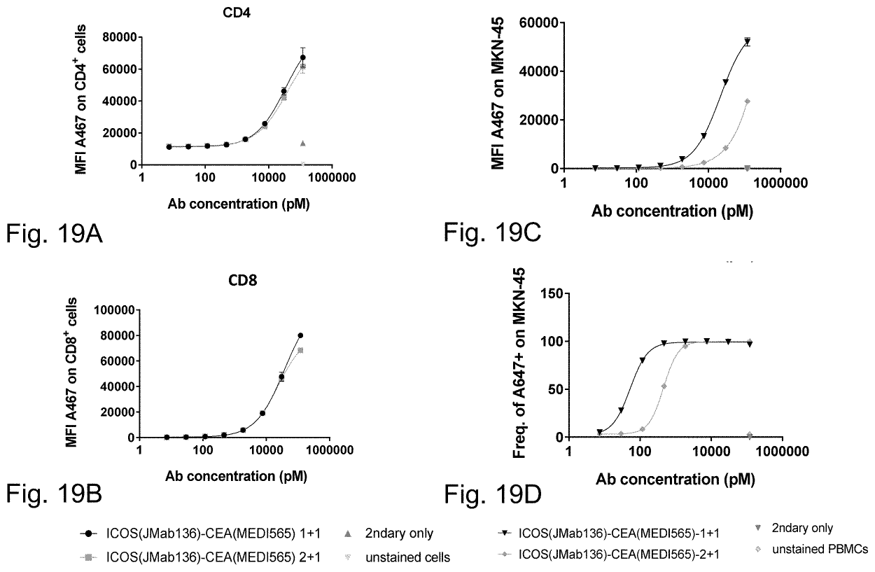

The results show that the CEA-ICOS molecules are able to bind to human ICOS on CD4⁺ and CD8⁺ T-cells (FIGS. 19A and 19B, respectively), as well as to human CEA in a concentration-dependent manner (FIGS. 19C and 19D). EC₅₀ values are depicted in Table 34. Both molecules exhibit similar binding profiles for human ICOS. However, the ICOS(JMab136)-CEA(MEDI565) 1+1 molecule showed better binding to human CEA as compared to the ICOS(JMab136)-CEA(MEDI565) 2+1 molecule, wherein the CEA is fused as VH-VL to the C-terminus of the Fc region. To see, if this translates into different functional activity, a co-culture assay with PBMC effector and CEA- as well as FAP-positive target cells was conducted (Example 20).

TABLE 34
EC₅₀ values of binding of different CEA-ICOS molecules
to ICOS⁺ PBMCs or CEA positive MKN-45 cells

| | EC₅₀ of binding (MFI) | | | |
|---|---|---|---|---|
| | Human ICOS | | Human CEA | |
| Molecules | CD4 | CD8 | MFI A647 | Freq A647+ |
| ICOS(JMab136)- | n.c. | n.c. | n.c. | 52.73 |
| CEA(MEDI565) 1 + 1 | | | | |
| ICOS(JMab136)- | n.c. | n.c. | n.c. | 454.3 |
| CEA(MEDI565) 2 + 1 | | | | |

### Example 20

### ICOS-Mediated Boosting of TCB-Induced T-Cell Activation (Flow Cytometry Analysis)

The capacity of either FAP- or CEA-targeted bispecific agonistic ICOS molecules to further boost CEACAM5-TCB-mediated activation of T-cells was assessed in a co-culture assay of CEA positive MKN-45 and FAP expressing NIH/3T3-huFAP cl.19 cells (ATCC, CCL-92, transfected to stably overexpress human FAP), as well as human PBMCs.

Briefly, adherent target cells were harvested with Cell Dissociation Buffer and plated at a density of 10 000 cells/well in flat-bottom 96-well plates one day before the experiment (Gibco, 13151014). Hereby, NIH/3T3-huFAP clone 19 cells were additionally irradiated before plating, using X-Ray Irradiator RS 2000 (Rad source) with 5000 rad (irradiation without filter, level 5). Target cells were left to adhere overnight. Peripheral blood mononuclear cells (PBMCs) were prepared by Histopaque (Sigma-Aldrich, Cat No. 10771-500ML Histopaque-1077) density centrifugation of enriched lymphocyte preparations from a Buffy Coat (“Blutspende Zirich”), as described in Example 5.1.

T-cell activation after 48 hours was determined upon co-incubation of PBMC effector and MKN45, as well as NIH3T3-hFAP target cells at a ratio of 5:1:1 in presence of a fixed concentration of 80 pM CEACAM5-TCB and increasing concentrations of the FAP- or CEA-targeted ICOS molecules (0.11 pM-5000 pM in triplicates), see Example 12. T-cell activation upon simultaneous therapy with targeted ICOS and TCB molecules was referenced to the TCB monotherapy.

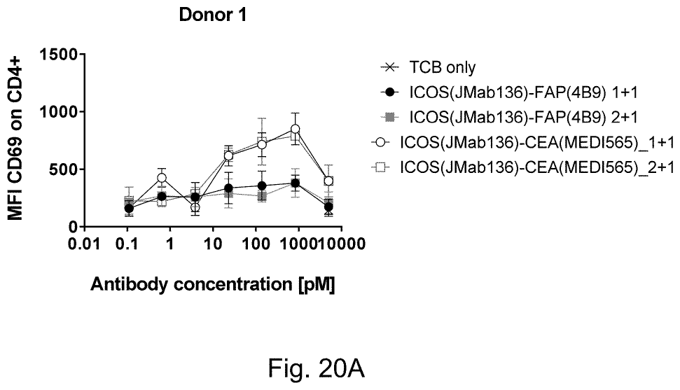

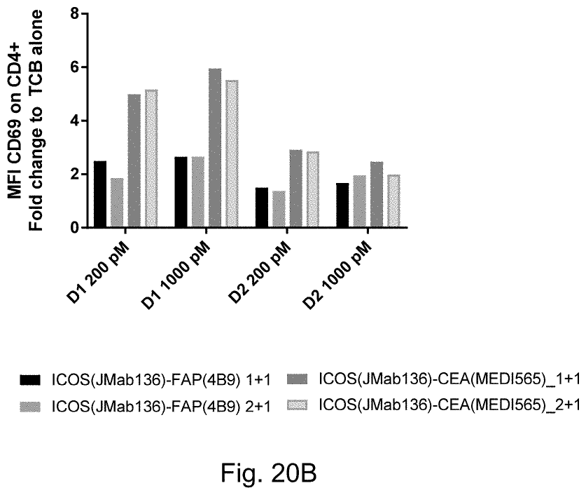

As shown in FIGS. 20A and 20B, both, the CEA-ICOS and the FAP-ICOS bispecific molecules, are able to further increase TCB-mediated activation compared to the monotherapy with CEACAM5-TCB treatment, as determined by up-regulation of the activation marker CD69 on CD4+ T-cells. As highlighted in FIG. 20A, the effect is concentration-dependent and decreases at high concentration of the ICOS molecule. Fold increase relative to TCB monotherapy is depicted in Table 35.

The CEA-ICOS molecules are clearly superior in further boosting TCB-mediated T-Cell activation as compared to FAP-targeted molecules. Both formats are equally potent, independent of the targeting moiety. Fold increase of the combination therapy relative to the TCB monotherapy was depicted in FIG. 20B at the indicated concentrations of the CEA- or FAP-targeted 1+1 or 2+1 formats (two representative donors).

TABLE 35
Fold increase of induction of CD69 on CD4⁺ T-
cells upon combination therapy with a targeted ICOS
molecule and a TCB compared to the TCB monotherapy

| | Fold increase vs | | Fold increase vs | |
|---|---|---|---|---|
| | TCB only at | | TCB only at | |
| | 1000 pM CEA-ICOS | | 200 pM CEA-ICOS | |
| | (% CD69⁺CD4⁺) | | (% CD69⁺CD4⁺) | |
| | Donor 1 | Donor 2 | Donor 1 | Donor 2 |
| Molecule | (D1) | (D2) | (D1) | (D2) |
| ICOS(JMab136)- | 2.65 | 1.68 | 2.50 | 1.50 |
| FAP(4B9) 1 + 1 | | | | |
| ICOS(JMab136)- | 2.66 | 1.96 | 2.66 | 1.38 |
| FAP(4B9) 2 + 1 | | | | |
| ICOS(JMab136)- | 5.95 | 2.47 | 4.99 | 2.91 |
| CEA(MEDI565) 1 + 1 | | | | |
| ICOS(JMab136)- | 5.52 | 1.99 | 5.17 | 2.86 |
| CEA(MEDI565) 2 + 1 | | | | |

### Example 21

### Binding of Murine ICOS Molecules to Cells Expressing Either Murine ICOS or Human CEA (Flow Cytometry Analysis)

The binding of several ICOS molecules prepared as described in Examples 17 or 18 was tested using murine ICOS-positive CHO (ATCC, CCL-61, transfected to stably overexpress murine ICOS), FAP positive NIH/3T3-moFAP cl.34 cells (ATCC, CCL-92, transfected to stably overexpress murine FAP), or CEA-positive MKN45 cells.

Briefly, adherent MKN45, NIH/3T3-moFAP or CHO-mICOS cells were detached using trypsin (Gibco, 25300-054), washed and 0.1 million cells were transferred into a 96-round bottom well-plate. Subsequent staining was performed for 30 min at 4° C. with increasing concentrations of CEA-ICOS/FAP-ICOS (3 pM-200 nM). Thereafter, cells were washed twice with cold PBS 0.1% BSA and further incubated for further 30 min at 4° C. with a labeled secondary antibody (PE-conjugated, #715-116-150 from Jackson Immuno Research Lab, diluted 1 to 50 in FACS buffer). Cells were washed with FACS buffer and the staining was fixed for 20 min at 4° C. in the dark, using 75 μl of 1% PFA in FACS buffer per well. Cells were washed twice with FACS Buffer and re-suspended in FACS Buffer for analysis.

Fluorescence was analyzed by FACS using a FACS Fortessa (Software FACS Diva). Binding curves and EC₅₀ values were obtained using GraphPadPrism6.

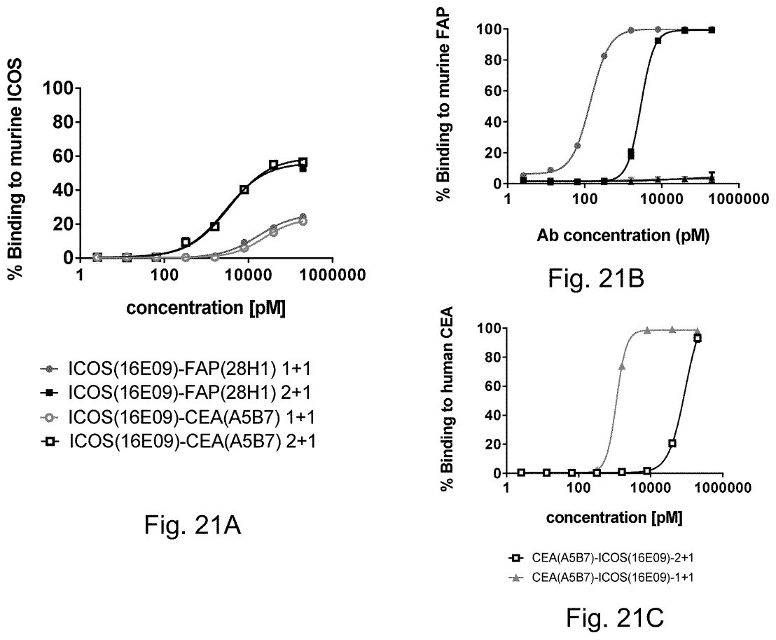

As shown in FIGS. 21A to 21C, the CEA- and FAP-targeted anti-murine ICOS molecules are able to bind to murine ICOS and human CEA or murine FAP, respectively, in a concentration-dependent manner. EC₅₀ values are depicted in Table 36. As expected, the 2+1 bispecific format with bivalent targeting to murine ICOS exhibits better binding than the 1+1 format with monovalent binding to murine ICOS. Binding of ICOS(16E09)-FAP(28H1) 2+1 respective ICOS(16E09)-CEA(A5B7) 2+1 to their respective target murine FAP or human CEA is weaker compared to the corresponding 1+1 formats. This is in line with the binding potency observed for the human molecules and can be explained by weaker affinity of the targeting moiety when it is fused as VH-VL compared to the 1+1 format, where it is included as Fab arm.

TABLE 36
EC₅₀ values of binding of different murine ICOS molecules targeted
to either murine FAP or human CEA expressed on cells (FACS)

| EC₅₀ | ICOS(16E09)- | ICOS(16E09)- | ICOS(16E09)- | ICOS(16E09)- |
|---|---|---|---|---|
| [pM] | FAP(28H1) 2 + 1 | FAP(28H1) 1 + 1 | CEA(A5B7) 2 + 1 | CEA(A5B7) 1 + 1 |
| Murine ICOS | 2882 | 17091 | 3443 | 23238 |
| Murine FAP | 2862 | 141.6 | n.d. | n.d. |
| Human CEA | n.d. | n.d. | 90947 | 1118 |

### Example 22

### ICOS-Mediated Boosting of TCB-Induced T-Cell Activation (Flow Cytometry Analysis)

The capacity of either FAP- or CEA-targeted bispecific agonistic ICOS molecules to further boost murine CEA-TCB-mediated activation of murine T-cells was assessed in a co-culture assay of CEA positive MC38-hCEA (Cancer Res 1975, 35:2434-2440) and murine (and human) FAP expressing NIH/3T3-huFAP clone 19 cells (ATCC, CCL-92, transfected to stably overexpress human FAP), as well as murine splenocytes from C57Bl/6 mice. As negative controls, DP47-targeted versions were included, which do not bind to any of the cells included in the assay. T-cell activation was assessed as up-regulation of CD69 on murine CD4⁺ and CD8⁺ T-cells after 48 h.

Briefly, adherent target cells were harvested with cell dissociation buffer and plated at density of 10 000 cells/well using flat-bottom 96-well plates one day before the experiment (Gibco, 13151014). NIH/3T3-huFAP clone 19 cells were additionally irradiated before plating using X-Ray Irradiator RS 2000 (Rad source) with 5000 rad (irradiation without filter, level 5), washed. Cells were left to adhere overnight. C57Bl/6 splenocytes were isolated transferring the spleen of C57Bl/6 mouse into GentleMACS C-tube (Miltenyi) filled with MACS buffer (PBS+0.5% BSA+2 mM EDTA). Spleens were dissociated using the GentleMACS Dissociator, tubes were spun down shortly and cells were passed through a 100 μm nylon cell strainer. Thereafter, tubes were rinsed with 3 ml RPMI1640 medium (SIGMA, Cat-No. R7388) and centrifuged for 8 min at 350×g. The supernatant was discarded, the cell suspension passed through a 70 μm nylon cell strainer and washed with medium. After another centrifugation (350×g, 8 min), supernatants were discarded and 5 ml ACK Lysis Buffer was added. After 5 min incubation at RT cells were washed with RPMI medium.

Afterwards the cells were re-suspended in assay medium (RPMI1640, containing 10% FCS, 1% GlutaMax (Gibco #35050061), 1 μM Sodium pyruvate (Gibco #11360070), 1 μM β-Mercaptoethanol (SIGMA, # M3148-100), 1× non-essential amino acids (NEAA, SIGMA, Cat.-No. M7145), 1× antibiotic—antimycotic (Gibco #15240062), 100 U/ml IL-2), for cell counting (Vi-Cell-Settings leukocytes, 1:10 dilution).

Murine T-cell activation was determined upon co-incubation of splenocytes, CEA-positive tumor and murine FAP-positive fibroblast cells at a final ratio of 3:1:1. for 48 h at 37° C., 5% CO₂ by flow cytometric analysis, using antibodies recognizing the T cell activation marker CD69 (early activation marker). Murine FAP-ICOS or CEA-ICOS molecules were added at the indicated concentrations (range of 5 pM-75000 pM in triplicates) and the murine CEA-TCB was added at a fixed concentration (1.5 nM). T-cell activation upon simultaneous therapy with targeted ICOS and TCB molecules was referenced to the TCB monotherapy.

Briefly, splenocytes were centrifuged at 400×g for 4 min and washed twice with phosphate buffered saline (PBS) containing 0.1% BSA (FACS buffer). Surface staining for CD45 (A1F1700 anti mouse CD45, BioLegend, #103132), CD8 (Bv711 anti-mouse CD8a, BioLegend #100747), CD4 (Bv421 anti-mouse CD4, BioLegend #100438), CD69 (Pe/Cy7 anti-mouse CD69, BioLegend #104512) was performed according to the suppliers' indications. Cells were washed twice with 150 μl/well PBS containing 0.1% BSA and fixed for 15-30 min at 4° C. using 75 μl/well FACS buffer, containing 1% PFA. After centrifugation, the samples were re-suspended in 150 μl/well FACS buffer and analyzed using BD FACS Fortessa.

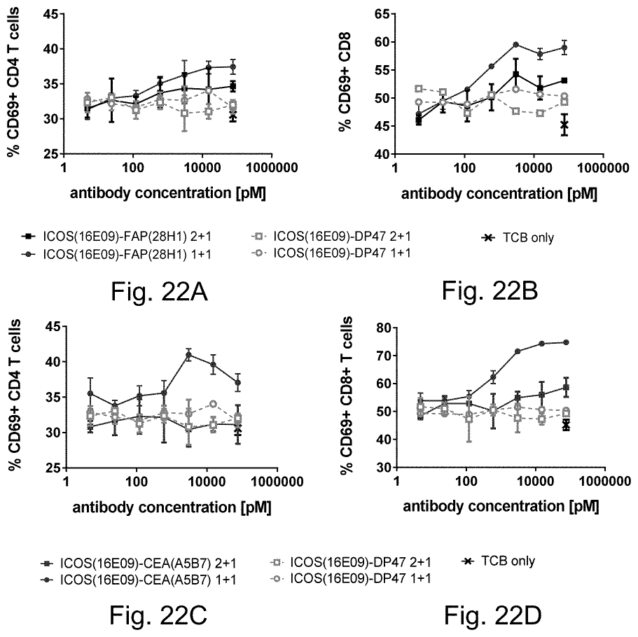

FIGS. 22A to 22D show that both, the CEA- and FAP-targeted anti-murine ICOS molecules, are able to further increase TCB-mediated activation compared to the monotherapy with muCEA-TCB treatment, as determined by up-regulation of the activation marker CD69 on murine CD4⁺ or CD8⁺ T-cells. The effect of the CEA-targeted 1+1 on CD4⁺ T cells is concentration-dependent, and decreases at high concentration of the CEA-targeted 1+1 ICOS molecule (like it was observed for the respective human molecules above). Activation depended on the molecules being crosslinked by simultaneous binding to ICOS and either CEA or FAP, since the DP47-targeted reference versions did not significantly boost any further activation of TCB-activated T-cells. Fold increase relative to TCB monotherapy is depicted in Table 37.

TABLE 37
Fold increase of induction of CD69 on murine CD4⁺ and
CD8⁺ T-cells upon combination therapy with a targeted
ICOS molecule and a TCB compared to the TCB monotherapy (FACS analysis)

| | Fold increase vs TCB only at | | Fold increase vs TCB only at | |
|---|---|---|---|---|
| | 3 nM CEA- or FAP-ICOS | | 75 nM CEA- or FAP-ICOS | |
| Molecule | % CD69⁺CD4⁺ | % CD69⁺CD8⁺ | % CD69⁺CD4⁺ | % CD69⁺CD8⁺ |
| ICOS(16E09)- | 1.1 | 2.3 | 1.1 | 2.4 |
| FAP(28H1) 2 + 1 | | | | |
| ICOS(16E09)- | 1.2 | 1.9 | 1.2 | 1.9 |
| FAP(28H1) 1 + 1 | | | | |
| ICOS(16E09)- | 1.0 | 1.8 | 1.0 | 1.9 |
| CEA(A5B7) 2 + 1 | | | | |
| ICOS(16E09)- | 1.3 | 1.8 | 1.2 | 1.7 |
| CEA(A5B7) 1 + 1 | | | | |

The CEA-ICOS bispecific molecules are superior in further boosting TCB-mediated T-Cell activation as compared to FAP-targeted molecules. For the molecules, targeting human ICOS, both formats are equally potent, independent of the targeting moiety.

### REFERENCES

Allen F., Bobanga J., et al., CCL3 in the tumor microenvironment augments the antitumor immune response. J Immunol May 1, 2016, 196 (75.11)
Allison J, Sharma P, Quezada, S. A., Fu T. Combination immunotherapy for the treatment of cancer, WO2011/041613A2 2009
Bacac M, Fauti T, Sam J, et al. A Novel Carcinoembryonic Antigen T-Cell Bispecific Antibody (CEA TCB) for the Treatment of Solid Tumours. Clin Cancer Res. 2016 Jul. 1; 22(13):3286-97.
Bacac M, Klein C, Umana P. CEA TCB: A novel head-to-tail 2:1 T cell bispecific antibody for treatment of CEA-positive solid tumours. Oncoimmunology. 2016 Jun. 24; 5(8).
Carthon B C et al., “Preoperative CTLA-4 blockade: Tolerability and immune monitoring in the setting of a presurgical clinical trial Clin Cancer Res. 2010 16(10); 2861-71.
Dammeijer F., Lau S. P., van Eijck C. H. J., van der Burg S. H., Aerts J. G. J. V. Rationally combining immunotherapies to improve efficacy of immune checkpoint blockade in solid tumours. Cytokine & Growth Factor Reviews 2017 August; 36: 5-15.
Davidson E. H., Hood L., Dimitrov K., Direct multiplexed measurement of gene expression with color-coded probe pairs. Nature biotechnology 2008; 26:317-325.
Fu T et al., The ICOS/ICOSL pathway is required for optimal antitumour responses mediated by anti-CTLA-4 therapy. Cancer Res. 2011, 71(16); 5445-54.
Geiss G. K. et al., Direct multiplexed measurement of gene expression with color-coded probe pairs. Nat Biotechnol. 2008 March; 26(3):317-25.
Giacomo A M D et al., “Long-term survival and immunological parameters in metastatic melanoma patients who respond to ipilimumab 10 mg/kg within an expanded access program”, Cancer Immunol Immunother. 2013, 62(6); 1021-8.
Gu-Trantien C., Migliori E., et al., CXCL13-producing T_{FH} cells link immune suppression and adaptive memory in human breast cancer. JCI Insight. 2017 Jun. 2; 2(11).
Hutloff A., Dittrich A. M., Beier K. C., Eljaschewitsch B., Kraft R., Anagnostopoulos I., Kroczek R. A. ICOS is an inducible T-cell co-stimulator structurally and functionally related to CD28. Nature. 1999 Jan. 21; 397(6716):263-6.
Im S. J., Hashimoto M., et al., Defining CD8+ T cells that provide the proliferative burst after PD-1 therapy. Nature. 2016 Sep. 15; 537(7620): 417-421.
Liakou C I et al., CTLA-4 blockade increases IFN-gamma producing CD4+ICOShi cells to shift the ratio of effector to regulatory T cells in cancer patients. Proc Natl Acad Sci USA 2008, 105(39); 14987-92.
Manzoor A. M., Developing Costimulatory Molecules for Immunotherapy of Diseases, Academic Press, 2015; eBook ISBN 9780128026755
Paulos C. M., Carpenito C., Plesa G., Suhoski M. M., Varela-Rohena A., Golovina T. N., Carroll R. G., Riley J. L., June C. H. The inducible costimulator (ICOS) is critical for the development of human T(H)17 cells. Sci Transl Med. 2010 Oct. 27; 2(55):55ra78.
Sharma P, Allison J 2015. The future of immune checkpoint therapy. Science 2015; 348: 56-61
Simpson T. R., Quezada S. A., Allison J. P. Regulation of CD4 T cell activation and effector function by inducible costimulator (ICOS). Current Opinion in Immunology 2010, 22.
Vonderheide R H et al., Tremelimumab in combination with exemestane in patients with advanced breast cancer and treatment-associated modulation of inducible costimulator expression on patient T cells, Clin Cancer Res. 2010, 16(13); 3485-94.
Wakamatsu E., Mathis D., Benoist C. Convergent and divergent effects of costimulatory molecules in conventional and regulatory CD4+ T cells. Proc Natl Acad Sci USA. 2013 Jan. 15; 110(3):1023-8.
Warnatz K., et al., Human ICOS deficiency abrogates the germinal center reaction and provides a monogenic model for common variable immunodeficiency. Blood 2006 107:3045-3052
Yao S., Zhu Y., Zhu G., Augustine M., Zheng L., Goode D. J., Broadwater M., Ruff W., Flies S., Xu H., Flies D., Luo L., Wang S., Chen L. B7-h2 is a costimulatory ligand for CD28 in human. Immunity. 2011 May 27; 34(5):729-40.
Young, M. R. I., Th17 Cells in Protection from Tumor or Promotion of Tumor Progression. J Clin Cell Immunol. 2016 June; 7(3): 431.
Yuraszeck et al., Translation and Clinical Development of Bispecific T-cell Engaging Antibodies for Cancer Treatment. Clinical Pharmacology & Therapeutics 2017, 101

1. A method of treating or delaying the progression of a cancer comprising administering to a subject:
(a) an agonistic ICOS-binding molecule that comprises at least one antigen-binding domain that specifically binds a tumor-associated antigen (“agICOS/TAA binding molecule”), and
(b) a T-cell activating anti-CD3 bispecific antibody that is capable of specifically binding a tumor-associated antigen (“TAA/CD3 antibody”).

2. The method of claim 1, wherein the tumor-associated antigen of the agICOS/TAA binds the same antigen as the tumor-associated antigen of the TAA/CD3 antibody.

3. The method of claim 1, wherein the tumor associated antigen of the agICOS/TAA binds a different antigen as the tumor-associated antigen of the TAA/CD3 antibody.

4. The method of claim 1, wherein the agICOS/TAA binding molecule and the TAA/CD3 antibody are administered together in a single composition or administered separately in two or more different compositions.

5. The method of claim 1, wherein the agICOS/TAA binding molecule comprises at least one antigen binding domain that is capable of specifically binding at least one ICOS-L, wherein the ICOS-L comprises an amino acid sequence of SEQ ID NO: 1 or SEQ ID NO:2.

6. The method of claim 1, wherein the agICOS/TAA binding molecule comprises at least one antigen binding domain that specifically and agonistically binds human ICOS comprising an amino acid sequence of SEQ ID NO:3.

7. The method of claim 1, wherein the agICOS/TAA binding molecule comprises at least one antigen binding domain that is capable of specifically binding Fibroblast activation protein (“FAP”).

8. The method of claim 7, wherein the at least one antigen binding domain of the agICOS/TAA binding molecule that is capable of specifically binding FAP comprises:
(a)
a heavy chain variable region (V_{H}FAP) comprising:
(i) a CDR-H1 comprising the amino acid sequence of SEQ ID NO:4,
(ii) a CDR-H2 comprising the amino acid sequence of SEQ ID NO:5, and
(iii) a CDR-H3 comprising the amino acid sequence of SEQ ID NO:6,
and a light chain variable region (V_{L}FAP) comprising:
(iv) a CDR-L1 comprising the amino acid sequence of SEQ ID NO:7,
(v) a CDR-L2 comprising the amino acid sequence of SEQ ID NO:8, and
(vi) a CDR-L3 comprising the amino acid sequence of SEQ ID NO:9; or
(b)
a heavy chain variable region (V_{H}FAP) comprising:
(i) a CDR-H1 comprising the amino acid sequence of SEQ ID NO: 12,
(ii) a CDR-H2 comprising the amino acid sequence of SEQ ID NO: 13, and
(iii) a CDR-H3 comprising the amino acid sequence of SEQ ID NO: 14,
and a light chain variable region (V_{L}FAP) comprising:
(iv) a CDR-L1 comprising the amino acid sequence of SEQ ID NO: 15,
(v) a CDR-L2 comprising the amino acid sequence of SEQ ID NO: 16, and
(vi) a CDR-L3 comprising the amino acid sequence of SEQ ID NO: 17.

9. The method of claim 7, wherein the at least one antigen binding domain of the agICOS/TAA binding molecule that is capable of specifically binding FAP comprises:
(a) a heavy chain variable region (V_{H}FAP) comprising an amino acid sequence of SEQ ID NO: 10 and a light chain variable region (V_{L}FAP) comprising an amino acid sequence of SEQ ID NO:11; or
(b) a heavy chain variable region (V_{H}FAP) comprising an amino acid sequence of SEQ ID NO: 18 and a light chain variable region (V_{L}FAP) comprising an amino acid sequence of SEQ ID NO:19.

10. The method of claim 1, wherein the agICOS/TAA binding molecule comprises at least one antigen binding domain that is capable of specifically binding Carcinoembryonic antigen (“CEA”).

11. The method of claim 10, wherein the at least one antigen binding domain of the agICOS/TAA binding molecule that is capable of specifically binding CEA comprises:
(a)
a heavy chain variable region (V_{H}CEA) comprising:
(i) a CDR-H1 comprising the amino acid sequence of SEQ ID NO: 145,
(ii) a CDR-H2 comprising the amino acid sequence of SEQ ID NO: 146, and
(iii) a CDR-H3 comprising the amino acid sequence of SEQ ID NO: 147,
and a light chain variable region (V_{L}CEA) comprising:
(iv) a CDR-L1 comprising the amino acid sequence of SEQ ID NO: 148,
(v) a CDR-L2 comprising the amino acid sequence of SEQ ID NO: 149, and
(vi) a CDR-L3 comprising the amino acid sequence of SEQ ID NO: 150; or
(b)
a heavy chain variable region (V_{H}CEA) comprising:
(i) a CDR-H1 comprising the amino acid sequence of SEQ ID NO: 158,
(ii) a CDR-H2 comprising the amino acid sequence of SEQ ID NO: 159, and
(iii) a CDR-H3 comprising the amino acid sequence of SEQ ID NO: 160,
and a light chain variable region (V_{L}CEA) comprising:
(iv) a CDR-L1 comprising the amino acid sequence of SEQ ID NO: 161,
(v) a CDR-L2 comprising the amino acid sequence of SEQ ID NO: 162, and
(vi) a CDR-L3 comprising the amino acid sequence of SEQ ID NO: 163.

12. The method of claim 10, wherein the at least one antigen binding domain of the agICOS/TAA molecule that is capable of specifically binding CEA comprises:
(a) a heavy chain variable region (V_{H}CEA) comprising an amino acid sequence of SEQ ID NO: 151 and a light chain variable region (V_{L}CEA) comprising an amino acid sequence of SEQ ID NO:152; or
(b) a heavy chain variable region (V_{H}CEA) comprising an amino acid sequence of SEQ ID NO: 164 and a light chain variable region (V_{L}CEA) comprising an amino acid sequence of SEQ ID NO:165.

13. The method of claim 1, wherein the agICOS/TAA binding molecule comprises at least one antigen binding domain that is capable of specifically binding ICOS, comprising (a) a heavy chain variable region (V_{H}ICOS) comprising:
(i) a CDR-H1 comprising the amino acid sequence of SEQ ID NO:20,
(ii) a CDR-H2 comprising the amino acid sequence of SEQ ID NO:21, and
(iii) a CDR-H3 comprising the amino acid sequence of SEQ ID NO:22,
and (b) a light chain variable region (V_{L}ICOS) comprising:
(i) a CDR-L1 comprising the amino acid sequence of SEQ ID NO:23,
(ii) a CDR-L2 comprising the amino acid sequence of SEQ ID NO:24, and
(iii) a CDR-L3 comprising the amino acid sequence of SEQ ID NO:25.

14. The method of claim 1, wherein the at least one antigen binding domain of the agICOS/TAA binding molecule that is capable of specifically binding ICOS comprises (a) a heavy chain variable region (V_{H}ICOS) comprising an amino acid sequence of SEQ ID NO:26 and a light chain variable region (V_{L}ICOS) comprising an amino acid sequence of SEQ ID NO:27.

15. The method of claim 1, wherein the agICOS/TAA binding molecule comprises a Fc domain, which Fc domain comprises at least one amino acid substitution that reduces at least one of Fc binding to an Fc receptor or effector function, or both Fc binding to an Fc receptor and effector function.

16. The method of claim 1, wherein the agICOS/TAA binding molecule comprises:
(a)
(i) a first heavy chain comprising an amino acid sequence of SEQ ID NO:28,
(ii) a first light chain comprising an amino acid sequence of SEQ ID NO:29,
(iii) a second heavy chain comprising an amino acid sequence of SEQ ID NO:30, and
(iv) a second light chain comprising an amino acid sequence of SEQ ID NO:31; or
(b)
(i) a first heavy chain comprising an amino acid sequence of SEQ ID NO:32,
(ii) a second heavy chain comprising an amino acid sequence of SEQ ID NO:66, and
(iii) one light chain comprising the amino acid sequence of SEQ ID NO:29.

17. The method of claim 1, wherein the agICOS/TAA binding molecule comprises:
(a) a first heavy chain comprising an amino acid sequence of SEQ ID NO: 155,
(b) a first light chain comprising an amino acid sequence of SEQ ID NO:29,
(c) a second heavy chain comprising an amino acid sequence of SEQ ID NO: 156, and
(d) a second light chain comprising an amino acid sequence of SEQ ID NO: 157.

18. The method of claim 1, wherein the TAA/CD3 antibody is an anti-CEA/anti-CD3 bispecific antibody.

19. The method of claim 18, wherein the anti-CEA/anti-CD3 bispecific antibody comprises
(a) a first antigen binding domain that is capable of specifically binding CD3 comprising a heavy chain variable region (V_{H}CD3) and a light chain variable region (V_{L}CD3), and
(b) a second antigen binding domain that is capable of specifically binding CEA comprising a heavy chain variable region (V_{H}CEA) and a light chain variable region (V_{L}CEA).

20. The method of claim 19, wherein the heavy chain variable region of the first antigen binding domain that is capable of specifically binding CD3 (V_{H}CD3) comprises an amino acid sequence of SEQ ID NO:40.

21. The method of claim 19, wherein the light chain variable region of the first antigen binding domain that is capable of specifically binding CD3 (V_{L}CD3) comprises an amino acid sequence of SEQ ID NO:41.

22. The method of claim 19, wherein
(a) the heavy chain variable region of the first antigen binding domain that is capable of specifically binding CD3 (V_{H}CD3) comprises an amino acid sequence of SEQ ID NO:40, and
(b) the light chain variable region of the first antigen binding domain that is capable of specifically binding CD3 (V_{L}CD3) comprises an amino acid sequence of SEQ ID NO:41.

23. A pharmaceutical product comprising
(a) a first composition comprising an agonistic ICOS-binding molecule comprising at least one antigen binding domain capable of specific binding to a tumor-associated antigen (“agICOS/TAA binding molecule”) and a pharmaceutically acceptable excipient; and
(b) a second composition comprising a T-cell activating anti-CD3 bispecific antibody specific for a tumor-associated antigen (“TAA/CD3 antibody”) and a pharmaceutically acceptable excipient.

24. The package of claim 23, further comprising a package insert that provides instructions for using the pharmaceutical composition to treat or delay the progression of cancer, in a subject.

25. The package of claim 24, wherein the package insert provides instruction for using the first and second compositions together, and either sequentially or simultaneously.

26. The method of claim 23, wherein the tumor-associated antigen of the agICOS/TAA binds the same antigen as the tumor-associated antigen of the TAA/CD3 antibody.

27. The method of claim 23, wherein the tumor associated antigen of the agICOS/TAA binds a different antigen as the tumor-associated antigen of the TAA/CD3 antibody.

28. An agonistic ICOS-binding molecule comprising at least one antigen binding domain capable of specific binding to a tumor-associated antigen (“agICOS/TAA binding molecule”), wherein the tumor-associated antigen is selected from the group consisting of Fibroblast activation protein (FAP), Carcinoembryonic antigen (CEA), Folate receptor alpha (FolR1), Melanoma-associated chondroitin sulfate proteoglycan (MCSP), Epidermal growth factor receptor (EGFR), human epidermal growth factor receptor 2 (HER2), and p95HER2.

29. The agICOS/TAA binding molecule of claim 28, wherein the at least one antigen binding domain that is capable of specifically binding FAP comprises:
(a)
a heavy chain variable region (V_{H}FAP) comprising:
(i) a CDR-H1 comprising the amino acid sequence of SEQ ID NO:4,
(ii) a CDR-H2 comprising the amino acid sequence of SEQ ID NO:5, and
(iii) a CDR-H3 comprising the amino acid sequence of SEQ ID NO:6, and
and a light chain variable region (V_{L}FAP) comprising:
(iv) a CDR-L1 comprising the amino acid sequence of SEQ ID NO:7,
(v) a CDR-L2 comprising the amino acid sequence of SEQ ID NO:8, and
(vi) a CDR-L3 comprising the amino acid sequence of SEQ ID NO:9;
or
(b)
a heavy chain variable region (V_{H}FAP) comprising:
(i) a CDR-H1 comprising the amino acid sequence of SEQ ID NO: 12,
(ii) a CDR-H2 comprising the amino acid sequence of SEQ ID NO: 13, and
(iii) a CDR-H3 comprising the amino acid sequence of SEQ ID NO: 14,
and a light chain variable region (V_{L}FAP) comprising:
(iv) a CDR-L1 comprising the amino acid sequence of SEQ ID NO: 15,
(v) a CDR-L2 comprising the amino acid sequence of SEQ ID NO: 16, and
(vi) a CDR-L3 comprising the amino acid sequence of SEQ ID NO: 17.

30. The agICOS/TAA binding molecule of claim 28, wherein the at least one antigen binding domain that is capable of specifically binding FAP comprises:
(a) a heavy chain variable region (V_{H}FAP) comprising an amino acid sequence of SEQ ID NO: 10 and a light chain variable region (V_{L}FAP) comprising an amino acid sequence of SEQ ID NO:11; or
(b) a heavy chain variable region (V_{H}FAP) comprising an amino acid sequence of SEQ ID NO: 18 and a light chain variable region (V_{L}FAP) comprising an amino acid sequence of SEQ ID NO:19.

31. The agICOS/TAA binding molecule of claim 28, wherein the at least one antigen binding domain that is capable of specifically binding CEA comprises:
(a)
a heavy chain variable region (V_{H}CEA) comprising:
(i) a CDR-H1 comprising the amino acid sequence of SEQ ID NO: 145,
(ii) a CDR-H2 comprising the amino acid sequence of SEQ ID NO: 146, and
(iii) a CDR-H3 comprising the amino acid sequence of SEQ ID NO: 147,
and a light chain variable region (V_{L}CEA) comprising:
(iv) a CDR-L1 comprising the amino acid sequence of SEQ ID NO: 148,
(v) a CDR-L2 comprising the amino acid sequence of SEQ ID NO: 149, and
(vi) a CDR-L3 comprising the amino acid sequence of SEQ ID NO: 150; or
(b)
a heavy chain variable region (V_{H}CEA) comprising:
(i) a CDR-H1 comprising the amino acid sequence of SEQ ID NO: 158,
(ii) a CDR-H2 comprising the amino acid sequence of SEQ ID NO: 159, and
(iii) a CDR-H3 comprising the amino acid sequence of SEQ ID NO: 160,
and a light chain variable region (V_{L}CEA) comprising:
(iv) a CDR-L1 comprising the amino acid sequence of SEQ ID NO: 161,
(v) a CDR-L2 comprising the amino acid sequence of SEQ ID NO: 162, and
(vi) a CDR-L3 comprising the amino acid sequence of SEQ ID NO: 163.

32. The agICOS/TAA binding molecule of claim 28, wherein the at least one antigen binding domain that is capable of specifically binding CEA comprises:
(a) a heavy chain variable region (V_{H}CEA) comprising an amino acid sequence of SEQ ID NO: 151 and a light chain variable region (V_{L}CEA) comprising an amino acid sequence of SEQ ID NO:152; or
(b) a heavy chain variable region (V_{H}CEA) comprising an amino acid sequence of SEQ ID NO: 164 and a light chain variable region (V_{L}CEA) comprising an amino acid sequence of SEQ ID NO:165.

33. The agICOS/TAA binding molecule of claim 28 that comprises at least one antigen binding domain that is capable of specifically binding ICOS, comprising
a heavy chain variable region (V_{H}ICOS) comprising:
(i) a CDR-H1 comprising the amino acid sequence of SEQ ID NO:20,
(ii) a CDR-H2 comprising the amino acid sequence of SEQ ID NO:21, and
(iii) a CDR-H3 comprising the amino acid sequence of SEQ ID NO:22,
and a light chain variable region (V_{L}ICOS) comprising:
(iv) a CDR-L1 comprising the amino acid sequence of SEQ ID NO:23,
(v) a CDR-L2 comprising the amino acid sequence of SEQ ID NO:24, and
(vi) a CDR-L3 comprising the amino acid sequence of SEQ ID NO:25.

34. The agICOS/TAA binding molecule of claim 28 that comprises monovalent binding to a tumor-associated target and monovalent binding to ICOS.

35. The agICOS/TAA binding molecule of claim 28 that comprises monovalent binding to a tumor-associated target and bivalent binding to ICOS.

36. An isolated polynucleotide encoding an agICOS/TAA binding molecule of claim 28.

37. A vector comprising the isolated polynucleotide of claim 36.

38. A host cell comprising the isolated polynucleotide of claim 36 or the vector of claim 37

39. A method for producing an agICOS/TAA binding molecule comprising culturing the host cell of claim 38 to express the agICOS/TAA binding molecule and recovering the expressed agICOS/TAA binding molecule.

40. A pharmaceutical composition comprising an agICOS/TAA binding molecule of claim 28 and at least one pharmaceutically acceptable excipient.

41. A method of treating or delaying the progression of a cancer comprising administering to a subject the agICOS/TAA binding molecule of claim 28 or the pharmaceutical composition of claim 40.

42. The method of claim 41, further comprising administering to the subject at least one of chemotherapeutic agent, radiation, or cancer immunotherapy, or a combination thereof.

43. The method of claim 42, wherein the cancer immunotherapy comprises an agent that blocks PD-L1 from interacting with PD-1.


SEQUENCE LISTING (176 sequences)

SEQ ID NO: 1 - protein, 238 aa, Homo sapiens [sequence omitted]

SEQ ID NO: 2 - protein, 233 aa, Mus musculus
ETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELK

SEQ ID NO: 3 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 4 - protein, 5 aa, Artificial Sequence
  FAP(4B9)  CDR-H1
SYAMS

SEQ ID NO: 5 - protein, 17 aa, Artificial Sequence
  FAP(4B9)  CDR-H2
AIIGSGASTYYADSVKG

SEQ ID NO: 6 - protein, 8 aa, Artificial Sequence
  FAP(4B9)  CDR-H3
GWFGGFNY

SEQ ID NO: 7 - protein, 12 aa, Artificial Sequence
  FAP(4B9)  CDR-L1
RASQSVSRSYLA

SEQ ID NO: 8 - protein, 7 aa, Artificial Sequence
  FAP(4B9)  CDR-L2
VGSRRAT

SEQ ID NO: 9 - protein, 9 aa, Artificial Sequence
  FAP(4B9)  CDR-L3
QQGIMLPPT

SEQ ID NO: 10 - protein, 117 aa, Artificial Sequence
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 11 - protein, 108 aa, Artificial Sequence
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 12 - protein, 5 aa, Artificial Sequence
  FAP(28H1)  CDR-H1
SHAMS

SEQ ID NO: 13 - protein, 16 aa, Artificial Sequence
  FAP(28H1)  CDR-H2
AIWASGEQYYADSVKG

SEQ ID NO: 14 - protein, 8 aa, Artificial Sequence
  FAP(28H1)  CDR-H3
GWLGNFDY

SEQ ID NO: 15 - protein, 12 aa, Artificial Sequence
  FAP(28H1)  CDR-L1
RASQSVSRSYLA

SEQ ID NO: 16 - protein, 7 aa, Artificial Sequence
  FAP(28H1)  CDR-L2
GASTRAT

SEQ ID NO: 17 - protein, 9 aa, Artificial Sequence
  FAP(28H1)  CDR-L3
QQGQVIPPT

SEQ ID NO: 18 - protein, 116 aa, Artificial Sequence
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 19 - protein, 108 aa, Artificial Sequence
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 20 - protein, 10 aa, Artificial Sequence
  ICOS  CDR-H1
GYTFTGYYMH

SEQ ID NO: 21 - protein, 17 aa, Artificial Sequence
  ICOS  CDR-H2
WINPHSGGTNYAQKFQG

SEQ ID NO: 22 - protein, 16 aa, Artificial Sequence
  ICOS  CDR-H3
TYYYDSSGYYHDAFDI

SEQ ID NO: 23 - protein, 11 aa, Artificial Sequence
  ICOS  CDR-L1
RASQGISRLLA

SEQ ID NO: 24 - protein, 7 aa, Artificial Sequence
  ICOS  CDR-L2
VASSLQS

SEQ ID NO: 25 - protein, 9 aa, Artificial Sequence
  ICOS  CDR-L3
QQANSFPWT

SEQ ID NO: 26 - protein, 125 aa, Artificial Sequence
  ICOS VH
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 27 - protein, 107 aa, Artificial Sequence
  ICOS VL
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 28 - protein, 453 aa, Artificial Sequence
  VHCH1(JMAb136)- Fc knob chain
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 29 - protein, 214 aa, Artificial Sequence
  VLCL(JMAb136) Light chain
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 438 aa, Artificial Sequence
  VHCH1 (4B9)- Fc hole chain
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 31 - protein, 224 aa, Artificial Sequence
  VLCL(4B9) Light chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 591 aa, Artificial Sequence
  VHCH1 (JMAb136)-Fc knob chain-VH (4B9)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 33 - protein, 582 aa, Artificial Sequence
  VHCH1 (JMAb136)-Fc hole chain-VL (4B9)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 34 - protein, 5 aa, Artificial Sequence
  CD3-HCDR1
TYAMN

SEQ ID NO: 35 - protein, 19 aa, Artificial Sequence
  CD3-HCDR2
RIRSKYNNYATYYADSVKG

SEQ ID NO: 36 - protein, 14 aa, Artificial Sequence
  CD3-HCDR3
HGNFGNSYVSWFAY

SEQ ID NO: 37 - protein, 14 aa, Artificial Sequence
  CD3-LCDR1
GSSTGAVTTSNYAN

SEQ ID NO: 38 - protein, 7 aa, Artificial Sequence
  CD3-LCDR2
GTNKRAP

SEQ ID NO: 39 - protein, 9 aa, Artificial Sequence
  CD3-LCDR3
ALWYSNLWV

SEQ ID NO: 40 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 41 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 42 - protein, 5 aa, Artificial Sequence
  CEA-HCDR1
EFGMN

SEQ ID NO: 43 - protein, 17 aa, Artificial Sequence
  CEA-HCDR2
WINTKTGEATYVEEFKG

SEQ ID NO: 44 - protein, 12 aa, Artificial Sequence
  CEA-HCDR3
WDFAYYVEAMDY

SEQ ID NO: 45 - protein, 11 aa, Artificial Sequence
  CEA-LCDR1
KASAAVGTYVA

SEQ ID NO: 46 - protein, 7 aa, Artificial Sequence
  CEA-LCDR2
SASYRKR

SEQ ID NO: 47 - protein, 10 aa, Artificial Sequence
  CEA-LCDR3
HQYYTYPLFT

SEQ ID NO: 48 - protein, 121 aa, Artificial Sequence
  CEA VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 49 - protein, 108 aa, Artificial Sequence
  CEA VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 50 - protein, 5 aa, Artificial Sequence
  CEA-HCDR1 (CEACAM5)
DTYMH

SEQ ID NO: 51 - protein, 17 aa, Artificial Sequence
  CEA-HCDR2 (CEACAM5)
RIDPANGNSKYVPKFQG

SEQ ID NO: 52 - protein, 12 aa, Artificial Sequence
  CEA-HCDR3 (CEACAM5)
FGYYVSDYAMAY

SEQ ID NO: 53 - protein, 15 aa, Artificial Sequence
  CEA-LCDR1 (CEACAM5)
RAGESVDIFGVGFLH

SEQ ID NO: 54 - protein, 7 aa, Artificial Sequence
  CEA-LCDR2 (CEACAM5)
RASNRAT

SEQ ID NO: 55 - protein, 9 aa, Artificial Sequence
  CEA-LCDR3 (CEACAM5)
QQTNEDPYT

SEQ ID NO: 56 - protein, 121 aa, Artificial Sequence
  CEA VH (CEACAM5)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 57 - protein, 111 aa, Artificial Sequence
  CEA VL (CEACAM5)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 58 - protein, 215 aa, Artificial Sequence
  Light chain CEA (CEA TCB)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 214 aa, Artificial Sequence
  Light chain CD3 (CEA TCB)
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - protein, 692 aa, Artificial Sequence
  CEA CD3 crossfab VHck fc knob P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 61 - protein, 449 aa, Artificial Sequence
  CEA VHCH1 Fc hole P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 62 - protein, 232 aa, Artificial Sequence
  CD3 VH-CL (CEACAM5 TCB)
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 449 aa, Artificial Sequence
  CEACAM5 VH-CH1(EE)-Fc (hole, P329G LALA)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 64 - protein, 674 aa, Artificial Sequence
  CEACAM5 VH-CH1(EE)-CD3 VL-CH1-Fc (knob, P329G LALA)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 65 - protein, 218 aa, Artificial Sequence
  CEACAM5  VL-CL(RK)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 354 aa, Artificial Sequence
  Fc hole chain-VL (4B9)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 67 - protein, 440 aa, Artificial Sequence
  VHCH1 (DP47)- Fc hole chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 222 aa, Artificial Sequence
  VLCL(DP47) Light chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 582 aa, Artificial Sequence
  VHCH1 (JMAb136)-Fc knob chain-VH (DP47)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 70 - protein, 589 aa, Artificial Sequence
  VHCH1 (JMAb136)-Fc hole chain-VL (DP47)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 71 - protein, 519 aa, Artificial Sequence
  Murine ICOSL Linker muIgG1 Fc (DAPG KK) 4GS linker FAP(28H1) VH
QDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKGSPGSSSSSGSADGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 72 - protein, 607 aa, Artificial Sequence
  Murine ICOSL linker muIgG1 Fc (DAPG DD) 4GS linker FAP(28H1) VL
ETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKGSPGSSSSSGSAGSPGSSSSSGSADGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 73 - protein, 607 aa, Artificial Sequence
  Murine ICOSL Linker muIgG1 Fc (DAPG KK) G4S linker DP47 VH
ETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKGSPGSSSSSGSAGSPGSSSSSGSADGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 74 - protein, 614 aa, Artificial Sequence
  Murine ICOSL linker muIgG1 Fc (DAPG DD) 4GS linker DP47 VL
ETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKGSPGSSSSSGSAGSPGSSSSSGSADGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 75 - protein, 443 aa, Artificial Sequence
  VHCH1(CH1A1A 98/99 2F1)- Fc(KK) DAPG chain
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 76 - protein, 215 aa, Artificial Sequence
  VLCL (CH1A1A 98/99 2F1) Light chain
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 77 - protein, 676 aa, Artificial Sequence
  VHCL VHCH1 (2C11- CH1A1A 98/99 2F1)- Fc(DD) DAPG chain
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSQVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSP

SEQ ID NO: 78 - protein, 211 aa, Artificial Sequence
  VLCH1 (2C11) Light chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 79 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 80 - protein, 748 aa, Artificial Sequence
  hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 81 - protein, 761 aa, murine
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 82 - protein, 749 aa, Artificial Sequence
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 83 - protein, 748 aa, Artificial Sequence
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 84 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 85 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 86 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 87 - protein, 257 aa, Cynomolgus
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 88 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 89 - protein, 1210 aa, Homo sapiens [sequence omitted]

SEQ ID NO: 90 - protein, 1255 aa, Homo sapiens [sequence omitted]

SEQ ID NO: 91 - protein, 645 aa, Homo sapiens
MPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 92 - protein, 5 aa, Artificial Sequence
  Peptide linker G4S
GGGGS

SEQ ID NO: 93 - protein, 10 aa, Artificial Sequence
  Peptide linker (G4S)2
GGGGSGGGGS

SEQ ID NO: 94 - protein, 10 aa, Artificial Sequence
  Peptide linker (SG4)2
SGGGGSGGGG

SEQ ID NO: 95 - protein, 14 aa, Artificial Sequence
  Peptide linker G4(SG4)2
GGGGSGGGGSGGGG

SEQ ID NO: 96 - protein, 10 aa, Artificial Sequence
  Peptide linker
GSPGSSSSGS

SEQ ID NO: 97 - protein, 15 aa, Artificial Sequence
  Peptide linker (G4S)3
GGGGSGGGGSGGGGS

SEQ ID NO: 98 - protein, 20 aa, Artificial Sequence
  Peptide linker (G4S)4
GGGGSGGGGSGGGGSGGGGS

SEQ ID NO: 99 - protein, 8 aa, Artificial Sequence
  Peptide linker
GSGSGSGS

SEQ ID NO: 100 - protein, 8 aa, Artificial Sequence
  Peptide linker
GSGSGNGS

SEQ ID NO: 101 - protein, 8 aa, Artificial Sequence
  Peptide linker
GGSGSGSG

SEQ ID NO: 102 - protein, 6 aa, Artificial Sequence
  Peptide linker
GGSGSG

SEQ ID NO: 103 - protein, 4 aa, Artificial Sequence
  Peptide linker
GGSG

SEQ ID NO: 104 - protein, 8 aa, Artificial Sequence
  Peptide linker
GGSGNGSG

SEQ ID NO: 105 - protein, 8 aa, Artificial Sequence
  Peptide linker
GGNGSGSG

SEQ ID NO: 106 - protein, 6 aa, Artificial Sequence
  Peptide linker
GGNGSG

SEQ ID NO: 107 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 108 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 109 - protein, 118 aa, Artificial Sequence [sequence omitted]

SEQ ID NO: 110 - protein, 107 aa, Artificial Sequence [sequence omitted]

SEQ ID NO: 111 - protein, 121 aa, Artificial Sequence
  VH (PD-L1) 2
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 112 - protein, 108 aa, Artificial Sequence
  VL (PD-L1) 2
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 113 - protein, 120 aa, Artificial Sequence [sequence omitted]

SEQ ID NO: 114 - protein, 111 aa, Artificial Sequence [sequence omitted]

SEQ ID NO: 115 - protein, 113 aa, Artificial Sequence [sequence omitted]

SEQ ID NO: 116 - protein, 107 aa, Artificial Sequence [sequence omitted]

SEQ ID NO: 117 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 118 - protein, 124 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWL

SEQ ID NO: 119 - DNA, 1071 bp, Artificial Sequence
  Nucleotide sequence murine ICOS antigen Fc hole chain
gagatcaacggcagcgccgaccaccggatgttcagcttccacaatggcggcgtgcagatcagctgcaagtaccccgagacagtgcagcagctgaagatgcggctgttccgcgagcgggaagtgctgtgcgagctgaccaagacaaagggcagcggcaacgccgtgtccatcaagaaccccatgctgtgcctgtaccacctgagcaacaacagcgtgtccttcttcctgaacaaccccgacagcagccagggcagctactacttctgctccctgagcatcttcgaccccccaccattccaggaacggaacctgagcggcggctacctgcacatctacgagagccagctgtgctgccagctgaaactgtggctgtctgcagacgtcgacgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccgcttcacgcagaagagcctctccctgtctccgggtaaatga

SEQ ID NO: 120 - DNA, 1170 bp, Artificial Sequence
  Nucleotide sequence murine ICOS antigen Fc knob chain
gagatcaacggcagcgccgaccaccggatgttcagcttccacaatggcggcgtgcagatcagctgcaagtaccccgagacagtgcagcagctgaagatgcggctgttccgcgagcgggaagtgctgtgcgagctgaccaagacaaagggcagcggcaacgccgtgtccatcaagaaccccatgctgtgcctgtaccacctgagcaacaacagcgtgtccttcttcctgaacaaccccgacagcagccagggcagctactacttctgctccctgagcatcttcgaccccccaccattccaggaacggaacctgagcggcggctacctgcacatctacgagagccagctgtgctgccagctgaaactgtggctgtctgcagacgtcgacgctagcggtggtagtccgacacctccgacacccgggggtggttctgcagacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgagtga

SEQ ID NO: 121 - protein, 356 aa, Artificial Sequence
  murine ICOS antigen Fc hole chain
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLSADVDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 122 - protein, 389 aa, Artificial Sequence
  murine ICOS antigen Fc knob chain
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLSADVDASGGSPTPPTPGGGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 123 - protein, 10 aa, Artificial Sequence
  Murine ICOS  CDR-H1
GYSFTSYWIG

SEQ ID NO: 124 - protein, 17 aa, Artificial Sequence
  Murine ICOS  CDR-H2
IIYPGDSDTRYSPSFQG

SEQ ID NO: 125 - protein, 11 aa, Artificial Sequence
  Murine ICOS  CDR-H3
SSGPYGLYLDY

SEQ ID NO: 126 - protein, 16 aa, Artificial Sequence
  Murine ICOS  CDR-L1
RSSQSLLHSNGYNYLD

SEQ ID NO: 127 - protein, 7 aa, Artificial Sequence
  Murine ICOS  CDR-L2
LGSNRAS

SEQ ID NO: 128 - protein, 9 aa, Artificial Sequence
  Murine ICOS  CDR-L3
MQALWTPTT

SEQ ID NO: 129 - protein, 120 aa, Artificial Sequence
  Murine ICOS (16E09) VH
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSS

SEQ ID NO: 130 - protein, 112 aa, Artificial Sequence
  Murine ICOS (16E09) VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALWTPTTFGQGTKVEIK

SEQ ID NO: 131 - protein, 219 aa, Artificial Sequence
  16E09 IgG1 light chain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALWTPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 449 aa, Artificial Sequence
  16E09 IgG1 P329GLALA heavy chain
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 389 aa, Artificial Sequence; identical to SEQ ID NO: 122
  murine ICOS Fc knob Avi-tag

SEQ ID NO: 134 - protein, 356 aa, Artificial Sequence; identical to SEQ ID NO: 121
  murine ICOS  Fc hole

SEQ ID NO: 135 - protein, 571 aa, Artificial Sequence
  VHCH1 (16E09) Fc DAPG DD heavy chain-VH (28H1)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 136 - protein, 579 aa, Artificial Sequence
  VHCH1 (16E09) Fc DAPG KK heavy chain-VL (28H1)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 137 - protein, 442 aa, Artificial Sequence
  VHCH1 (16E09) Fc DAPG DD heavy chain
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSP

SEQ ID NO: 138 - protein, 432 aa, Artificial Sequence
  VHCH1 (28H1) Fc DAPG KK heavy chain
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 139 - protein, 223 aa, Artificial Sequence
  VLCL (28H1)-light chain
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 140 - protein, 571 aa, Artificial Sequence
  VHCH1 (16E09) Fc DAPG DD heavy chain-VH (DP47)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 141 - protein, 578 aa, Artificial Sequence
  VHCH1 (16E09) Fc DAPG KK heavy chain-VL (DP47)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 142 - protein, 442 aa, Artificial Sequence; identical to SEQ ID NO: 137
  VHCH1 (16E09) Fc DAPG DD heavy chain

SEQ ID NO: 143 - protein, 442 aa, Artificial Sequence
  VHCH1 (DP47) Fc DAPG KK heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 144 - protein, 212 aa, Artificial Sequence
  Murine VLCL (DP47)-light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 145 - protein, 5 aa, Artificial Sequence
  CEA (MEDI-565)- CDR-H1
SYWMH

SEQ ID NO: 146 - protein, 16 aa, Artificial Sequence
  CEA (MEDI-565)- CDR-H2
FIRNKANGGTTEYAAS

SEQ ID NO: 147 - protein, 10 aa, Artificial Sequence
  CEA (MEDI-565)- CDR-H3
DRGLRFYFDY

SEQ ID NO: 148 - protein, 14 aa, Artificial Sequence
  CEA (MEDI-565)- CDR-L1
TLRRGINVGAYSIY

SEQ ID NO: 149 - protein, 11 aa, Artificial Sequence
  CEA (MEDI-565)- CDR-L2
YKSDSDKQQGS

SEQ ID NO: 150 - protein, 10 aa, Artificial Sequence
  CEA (MEDI-565)- CDR-L3
MIWHSGASAV

SEQ ID NO: 151 - protein, 121 aa, Artificial Sequence
  CEA (MEDI-565) VH
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 152 - protein, 116 aa, Artificial Sequence
  CEA (MEDI-565) VL
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 153 - protein, 590 aa, Artificial Sequence
  VHCH1 (JMAb136)-Fc knob chain-VH (MEDI-565)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 154 - protein, 585 aa, Artificial Sequence
  VHCH1 (JMAb136)-Fc hole chain-VL (MEDI-565)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 155 - protein, 453 aa, Artificial Sequence
  VHCH1(JMAb136)- Fc hole chain
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 156 - protein, 446 aa, Artificial Sequence
  VHCH1 (MEDI-565)-Fc knob chain
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 157 - protein, 228 aa, Artificial Sequence
  VLCL(MEDI-565) Light chain
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 5 aa, Artificial Sequence
  CEA (A5B7)- CDR-H1
DYYMN

SEQ ID NO: 159 - protein, 19 aa, Artificial Sequence
  CEA (A5B7)- CDR-H2
FIGNKANGYTTEYSASVKG

SEQ ID NO: 160 - protein, 10 aa, Artificial Sequence
  CEA (A5B7)- CDR-H3
DRGLRFYFDY

SEQ ID NO: 161 - protein, 10 aa, Artificial Sequence
  CEA (A5B7)- CDR-L1
RASSSVTYIH

SEQ ID NO: 162 - protein, 7 aa, Artificial Sequence
  CEA (A5B7)- CDR-L2
ATSNLAS

SEQ ID NO: 163 - protein, 9 aa, Artificial Sequence
  CEA (A5B7)- CDR-L3
QHWSSKPPT

SEQ ID NO: 164 - protein, 121 aa, Artificial Sequence
  CEA (A5B7) VH
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 165 - protein, 106 aa, Artificial Sequence
  CEA (A5B7) VL
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 166 - protein, 569 aa, Artificial Sequence
  VHCH1 (16E09) Fc DAPG DD heavy chain-VL (A5B7)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSQTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 167 - protein, 584 aa, Artificial Sequence
  VHCH1 (16E09) Fc DAPG KK heavy chain-VH (A5B7)
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSGPYGLYLDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 168 - protein, 448 aa, Artificial Sequence
  VHCH1 (A5B7) Fc DAPG KK heavy chain
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 169 - protein, 210 aa, Artificial Sequence
  VLCL (A5B7)-light chain
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 170 - protein, 98 aa, Artificial Sequence
  CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 171 - protein, 5 aa, Artificial Sequence
  linker
EPKSC

SEQ ID NO: 172 - protein, 10 aa, Artificial Sequence
  hinge region, X = S or P
  misc_feature (8)..(8): Xaa can be any naturally occurring amino acid
DKTHTCPXCP

SEQ ID NO: 173 - protein, 7 aa, Artificial Sequence
  short hinge region, X = S or P
  misc_feature (5)..(5): Xaa can be any naturally occurring amino acid
HTCPXCP

SEQ ID NO: 174 - protein, 5 aa, Artificial Sequence
  shortest hinge region, X = S or P
  misc_feature (3)..(3): Xaa can be any naturally occurring amino acid
CPXCP

SEQ ID NO: 175 - protein, 107 aa, Artificial Sequence
  CH2 domain
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 176 - protein, 105 aa, Artificial Sequence
  CH3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP